(19) United States
(12) Patent Application Publication
(10) Pub. No.: US 2013/0266636 A1
(43) Pub. Date: Oct. 10, 2013

(54) METHODS FOR BLOCKING CELL PROLIFERATION AND TREATING DISEASES AND CONDITIONS RESPONSIVE TO CELL GROWTH INHIBITION

(71) Applicants: David Cheresh, Encinitas, CA (US); Eric Murphy, San Marcos, CA (US); Ainhoa Mielgo, Liverpool (GB)

(72) Inventors: David Cheresh, Encinitas, CA (US); Eric Murphy, San Marcos, CA (US); Ainhoa Mielgo, Liverpool (GB)

(73) Assignee: THE REGENTS OF THE UNIVERSITY OF CALIFORNIA, Oakland, CA (US)

(21) Appl. No.: 13/816,218

(22) PCT Filed: Aug. 12, 2011
(86) PCT No.: PCT/US11/47540

§ 371 Date: Jun. 19, 2013

Related U.S. Application Data
(60) Provisional application No. 61/373,175, filed on Aug. 12, 2010.

(51) Int. Cl. A61K 31/506

(52) U.S. Cl. CPC A61K 31/506
USPC 424/450; 435/375; 514/256; 544/327; 435/15; 435/184; 435/7.4

(57) ABSTRACT

In alternative embodiments, the invention provides methods for regulating or modulating RAF kinases. In alternative embodiments, the invention provides methods for ameliorating, preventing and/or treating diseases, infections and/or conditions having unwanted, pathological or aberrant cell proliferation, or that are responsive to inhibition or arrest of cell growth, by administration of an allosteric RAF inhibitor and/or any agent which prevents localization of RAF to mitotic spindles or mid-bodies.

### RELATED APPLICATIONS

This application is a national phase application claiming benefit of priority under 35 U.S.C. §371 to Patent Convention Treaty (PCT) International Application Serial No: PCT/US2011/047540, filed Aug. 12, 2011, which claims benefit of priority to U.S. Provisional Patent Application Serial No. (USSN) 61/373,175, filed Aug. 12, 2010. The aforementioned applications are expressly incorporated herein by reference in their entirety and for all purposes.

### GOVERNMENT RIGHTS

This invention was made with government support under grant number 2R37CA050286 and 5R01CA045726 awarded by the National Institutes of Health (NIH). The government has certain rights in the invention.

### TECHNICAL FIELD

This invention generally relates to cell and molecular biology, diagnostics and oncology. In alternative embodiments, the invention provides methods for regulating or modulating RAF kinases. In alternative embodiments, the invention provides methods for ameliorating, preventing and/or treating diseases, infections and/or conditions having unwanted, pathological or aberrant cell proliferation, or that are responsive to inhibition or arrest of cell growth, by administration of an allosteric RAF (or CRAF) inhibitor and/or any agent which prevents localization of RAF to mitotic spindles or mid-bodies.

### BACKGROUND

RAF (Raf) is a serine/threonine protein kinase that phosphorylates the OH group of serine or threonine. c-Raf is a MAP kinase (MAP3K) which functions downstream of the Ras subfamily of membrane associated GTPases to which it binds directly (RAF proto-oncogene serine/threonine-protein kinase is also known as proto-oncogene c-RAF or simply c-Raf). Once activated Raf-1 can phosphorylate to activate the dual specificity protein kinases MEK1 and MEK2 which in turn phosphorylate to activate the serine/threonine specific protein kinases ERK1 and ERK2. RAF kinases play a role in tumorigenesis, and are associated with tumor metastasis, radiation and chemo-resistance, and angiogenesis.

Allosteric regulation is the regulation of an enzyme or other protein by binding an effector molecule (e.g., an inhibitor or activator) at the protein's allosteric site, i.e., a site other than the protein's active site.

One class of RAF inhibitors are ATP-competitive RAF kinase inhibitors (also known as Type I inhibitors) that block cell proliferation at the G1 or S phase of the cell cycle. However these ATP-mimetic inhibitors have recently been shown to be in some circumstances inefficient and to even promote cell proliferation instead of inhibiting it. ATP-competitive RAF inhibitors have side effects and in fact may promote cell proliferation.

### SUMMARY

In alternative embodiments, the invention provides methods for regulating or modulating cell growth or mitosis, angiogenesis, or regulating or modulating phospho-5338 CRAF localization to centrosomes/mitotic spindle poles, comprising

(1) (a) providing a composition comprising or consisting of:
(i) an allosteric regulator of a c-RAF kinase protein,
wherein the allosteric c-RAF regulator comprises an allosteric c-RAF inhibitor or an allosteric c-RAF activator, or
(ii) a composition or an agent which inhibits or prevents localization of a c-RAF to a mitotic spindle or mid-body; and

(b) administering a sufficient amount of the composition to the cell to regulate or modulate cell growth or mitosis,

wherein administering an allosteric c-RAF activator increases or unregulates cell growth or mitosis, or angiogenesis, or blocks G2M arrest,

and administering an allosteric c-RAF inhibitor, or a composition or an agent which inhibits or prevents localization of a c-RAF to a mitotic spindle, or inhibits, suppresses or decreases cell growth or mitosis, or inhibits or suppresses angiogenesis, or causes cell growth arrest,

and administering an allosteric c-RAF inhibitor inhibits phospho-5338 CRAF localization to centrosomes/mitotic spindle poles; or

(2) the method of (1), wherein the composition comprises a pharmaceutical composition administered in vivo; or

(3) the method of (1) or (2), wherein the composition is formulated for administration intravenously (IV), parenterally, orally, or by liposome or vessel-targeted nanoparticle delivery.

(4) the method of any of (1) to (3), wherein the allosteric c-RAF inhibitor is not an ATP-competitive RAF inhibitor;

(5) the method of any of (1) to (3), wherein the allosteric c-RAF inhibitor or composition or agent which inhibits or prevents localization of a c-RAF to a mitotic spindle, acts by promoting G2M arrest, wherein optionally the G2M arrest is effected by the allosteric c-RAF inhibitor blocking association of RAF with a mitotic spindle and/or a mid-body during cell mitosis;

(6) the method of any of (1) to (5), wherein administering an allosteric c-RAF inhibitor, or a composition or an agent which inhibits or prevents localization of a c-RAF to a mitotic spindle, decreases, slows or blocks cancer cell growth;

(7) the method of any of (1) to (6), wherein administering an allosteric c-RAF inhibitor, or a composition or an agent which inhibits or prevents localization of a c-RAF to a mitotic spindle, decreases, slows or blocks new blood vessel growth, neovascularization or angiogenesis; or

(8) the method of any of (1) to (7), wherein administering an allosteric c-RAF inhibitor, or a composition or an agent which inhibits or prevents localization of a c-RAF to a mitotic spindle, treats or ameliorates conditions that are responsive to blocking or slowing cell growth, and/or the development of neovascularization or new blood vessels,

wherein the method optionally reduces, treats or ameliorates the level of disease in a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

In alternative embodiments, the invention provides methods for reducing, treating or ameliorating a condition or disease responsive to slowing, decreasing the rate of, arresting or inhibiting cell growth, comprising:

(1) (a) providing a composition comprising or consisting of:
(i) an allosteric regulator of a c-RAF kinase protein,
wherein the allosteric c-RAF regulator comprises an allosteric c-RAF inhibitor or an allosteric c-RAF activator, or
(ii) a composition or an agent which inhibits or prevents localization of a c-RAF to a mitotic spindle; and

(b) administering a sufficient amount of the composition to the cell to regulate or modulate cell growth or mitosis,

wherein administering an allosteric c-RAF inhibitor, or a composition or an agent which inhibits or prevents localization of a c-RAF to a mitotic spindle, inhibits, suppresses or decreases cell growth or mitosis, or causes cell growth arrest,

wherein administering an allosteric c-RAF activator increases or unregulates cell growth or mitosis, or blocks G2M arrest; or

(2) the method of (1), wherein the composition comprises a pharmaceutical composition administered in vivo; or

(3) the method of (1) or (2), wherein the composition is formulated for administration intravenously (IV), parenterally, orally, or by liposome or vessel-targeted nanoparticle delivery.

(4) the method of any of (1) to (3), wherein the allosteric c-RAF inhibitor is not an ATP-competitive RAF inhibitor;

(5) the method of any of (1) to (3), wherein the allosteric c-RAF inhibitor or composition or agent which inhibits or prevents localization of a c-RAF to a mitotic spindle, acts by promoting G2M arrest, wherein optionally the G2M arrest is effected by the allosteric RAF inhibitor blocking association of c-RAF with a mitotic spindle and/or a mid-body during cell mitosis;

(6) the method of any of (1) to (5), wherein administering an allosteric c-RAF inhibitor, or a composition or an agent which inhibits or prevents localization of a c-RAF to a mitotic spindle, decreases, slows or blocks cancer cell growth;

(7) the method of any of (1) to (5), wherein administering an allosteric c-RAF inhibitor, or a composition or an agent which inhibits or prevents localization of a RAF to a mitotic spindle, decreases, slows or blocks new blood vessel growth, neovascularization or angiogenesis; or

(7) the method of any of (1) to (5), wherein administering an allosteric c-RAF inhibitor, or a composition or an agent which inhibits or prevents localization of a c-RAF to a mitotic spindle, treats or ameliorates conditions that are responsive to blocking or slowing cell growth, and/or the development of neovascularization or new blood vessels,

wherein the method optionally reduces, treats or ameliorates the level of disease in a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

In alternative embodiments, the invention provides products of manufacture or kits comprising a composition comprising or consisting of: (i) an allosteric regulator of a c-RAF kinase protein, wherein the allosteric c-RAF regulator comprises an allosteric c-RAF inhibitor or an allosteric c-RAF activator, or (ii) a composition or an agent which inhibits or prevents localization of a c-RAF to a mitotic spindle; and optionally further comprising instructions for practicing a method of the invention.

In alternative embodiments, the invention provides methods for determining whether an individual or a patient would benefit from administration of an allosteric c-RAF inhibitor, or determining the therapeutic efficacy of an agent that blocks cell proliferation at the mitotic phase, comprising:

(a) detecting a serine-338 phosphorylated c-RAF, or detecting a serine-338 phosphorylated c-RAF localized to the mitotic spindle, wherein optionally the detection is by analysis or visualization of a biopsy or other tissue sample or a pathology slide taken from the patient or individual,

wherein detection of a serine-338 phosphorylated c-RAF, or detection of a serine-338 phosphorylated c-RAF localized to the mitotic spindle, indicates: that the individual or patient will be responsive to the allosteric c-RAF inhibitor, or that the agent that blocks cell proliferation at the mitotic phase will have therapeutic efficacy; or

(b) the method of (a), wherein an antibody that can specifically bind to a serine-338 phosphorylated c-RAF is used to detect a serine-338 phosphorylated RAF, or to detect a serine-338 phosphorylated c-RAF localized to the mitotic spindle.

In alternative embodiments, the invention provides methods for blocking proliferating cells in mitosis by preventing c-RAF localization to a mitotic spindle or a mid-body of a mammalian cell comprising:

(1) (a) providing a composition comprising or consisting of:
(i) an allosteric c-RAF inhibitor of a c-RAF kinase protein, or
(ii) a composition or an agent which inhibits or prevents localization of a c-RAF to a mitotic spindle or a mid-body; and

(b) administering a sufficient amount of the composition to the mammalian cell to block proliferating mammalian cells in mitosis by preventing c-RAF localization to the mitotic spindle or mid-body.

In alternative embodiments, the invention provides methods for inhibiting or impairing a polo-like kinase 1 (Plk1 kinase) activity and/or inhibiting or impairing c-Plk1 kinase accumulation at a kinetochore in a mammalian cell comprising:

(1) (a) providing a composition comprising or consisting of:
(i) an allosteric c-RAF inhibitor of a c-RAF kinase protein, or
(ii) a composition or an agent which inhibits or prevents localization of a RAF to a mitotic spindle or a mid-body; and

(b) administering a sufficient amount of the composition to the mammalian cell to inhibit or impair the Plk1 kinase activity, or inhibit or impair Plk1 kinase accumulation at the kinetochore, in the mammalian cell.

In alternative embodiments, of the methods and/or the kits of the invention, the allosteric RAF inhibitor comprises or consists of (or is selected from the group consisting of) any one of the following compounds, or equivalents thereof:
(a) N-(3-(4-(pyridin-3-yl)pyrimidin-2-ylamino)-4-methylphenyl)-4-((4-methylpiperazin-1-yl)methyl)benzamide;
(b) 1-(4-(2-(methylcarbamoyl)pyridin-4-yloxy)phenyl)-3-(4-chloro-3-(trifluoromethyl)phenyl)urea;
(c) 6-(4-(5-(3-(trifluoromethyl)phenylamino)-4H-1,2,4-triazol-3-yl)phenoxy)pyrimidin-4-amine;
(d) 6-(4-(5-(3-(trifluoromethyl)phenylamino)-4H-1,2,4-triazol-3-yl)phenoxy)pyrimidine-2,4-diamine;
(e) 6-(4-(5-(4-chloro-3-(trifluoromethyl)phenylamino)-4H-1,2,4-triazol-3-yl)phenoxy)pyrimidine-2,4-diamine;
(f) 6-(4-(5-(3-(trifluoromethyl)phenylamino)-4H-1,2,4-triazol-3-yl)phenoxy)-2-(methylthio)pyrimidin-4-amine;
(g) 5-(4-(2-(methylthio)pyrimidin-4-yloxy)phenyl)-N-(3-(trifluoromethyl)phenyl)-4H-1,2,4-triazol-3-amine;
(h) 6-(4-(5-(4-chloro-3-(trifluoromethyl)phenylamino)-4H-1,2,4-triazol-3-yl)phenoxy)-2-(methylthio)pyrimidin-4-amine;
(i) 4-(4-(5-(3-(trifluoromethyl)phenylamino)-4H-1,2,4-triazol-3-yl)phenoxy)-6-methoxypyrimidin-2-amine;
(j) 6-(4-(5-(3-(trifluoromethyl)phenylamino)-1,3,4-oxadiazol-2-yl)phenoxy)pyrimidine-2,4-Diamine;
(k) 6-(4-(5-(3-(trifluoromethyl)phenylamino)-1,3,4-oxadiazol-2-yl)phenoxy)-2-(methylthio)pyrimidin-4-amine;
(l) (Z)-3-((3,5-dimethyl-1 Hpyrrol-2-yl)methylene)indolin-2-one;
(m) 3-(3,5-dibromo-4-hydroxybenzylidene)-5-iodoindolin-2-one;
(n) 2-(methylsulfanyl)-6-[4-(2-{[3-(trifluoromethyl)phenyl]amino}-1H-imidazol-5-yl)phenoxy]pyrimidin-4-amine (also called “KG5”), or 2-(methylsulfanyl)-6-[4-(2-{[3-(trifluoromethyl)phenyl]amino}-1H-imidazol-5-yl)phenoxy]pyrimidin-4-amine (also called “K38-B, or KG38”), having the structure:

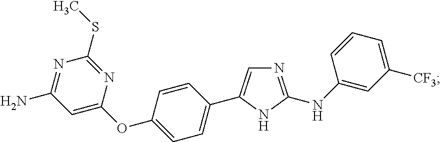 [SMILES: CSc1nc(N)cc(Oc2ccc(-c3cnc(Nc4cccc(C(F)(F)F)c4)[nH]3)cc2)n1]

(o) 6-(4-{5-[(2,3-dimethylphenyl)amino]-4H-1,2,4-triazol-3-yl}phenoxy)-2-(methylsulfanyl)pyrimidin-4-amine (also called “H3-11”), having the structure:

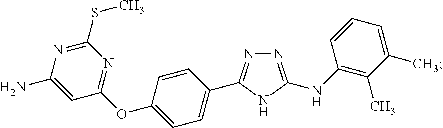 [SMILES: CSc1nc(N)cc(Oc2ccc(-c3nnc(Nc4cccc(C)c4C)[nH]3)cc2)n1]

(p) 2-(methylsulfanyl)-6-{4-[5-(5,6,7,8-tetrahydronaphthalen-1-ylamino)-4H-1,2,4-triazol-3-yl]phenoxy}pyrimidin-4-amine (also called “H3-21”), having the structure:

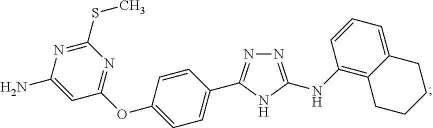 [SMILES: CSc1nc(N)cc(Oc2ccc(-c3nnc(Nc4cccc5c4CCCC5)[nH]3)cc2)n1]

(q) 6-(4-{5-[(3,4-dimethylphenyl)amino]-4H-1,2,4-triazol-3-yl}phenoxy)-2-(methylsulfanyl)pyrimidin-4-amine (also called “H3-9”), having the structure:

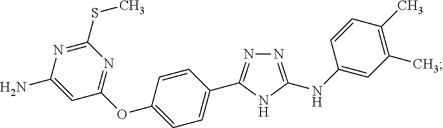 [SMILES: CSc1nc(N)cc(Oc2ccc(-c3nnc(Nc4ccc(C)c(C)c4)[nH]3)cc2)n1]

(r) 6-(4-{5-[(3,5-dimethylphenyl)amino]-4H-1,2,4-triazol-3-yl}phenoxy)-2-(methylsulfanyl)pyrimidin-4-amine (also called “H3-7”), having the structure:

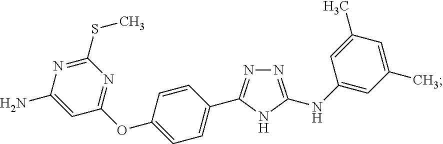 [SMILES: CSc1nc(N)cc(Oc2ccc(-c3nnc(Nc4cc(C)cc(C)c4)[nH]3)cc2)n1]

(s) 6-(4-{5-[(2,5-dimethylphenyl)amino]-4H-1,2,4-triazol-3-yl}phenoxy)-2-(methylsulfanyl)pyrimidin-4-amine (also called “H3-3”), having the structure:

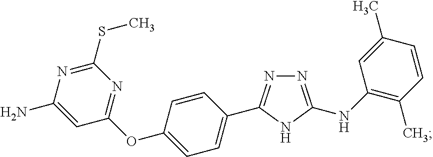 [SMILES: CSc1nc(N)cc(Oc2ccc(-c3nnc(Nc4cc(C)ccc4C)[nH]3)cc2)n1]

(t) 2-ethoxy-6-[4-(5-{[3-(trifluoromethyl)phenyl]amino}-4H-1,2,4-triazol-3-yl)phenoxy]pyrimidin-4-amine (also called “K1-72”), having the structure:

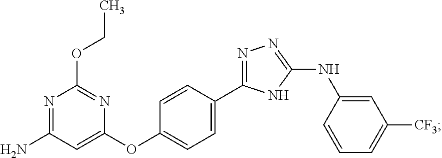 [SMILES: CCOc1nc(N)cc(Oc2ccc(-c3nnc(Nc4cccc(C(F)(F)F)c4)[nH]3)cc2)n1]

(u) a compound having a structural Formula I:

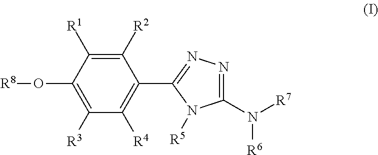

or a pharmaceutically acceptable salt or solvate thereof,

wherein:
R¹-R⁶ are independently selected from the group consisting of hydrogen, deuterium, alkyls, alkenyls, alkynyls, aryls, hetero-alkyls, hetero-alkenyls, hetero-alkynyls, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, thiols, sulfhydryls, sulfides, disulfides, sulfinyls, sulfos, thiocyanates, isothiocyanates, carbonothioyls, phosphinos, phosphonos, phosphates, silyls, and Si(OH)₃; and
R⁷-R⁸ are independently selected from the group consisting of cycloalkyl, cycloalkenyl, cycloalkynyl, aryl, heterocyle, and extended ring system; and/or
(v) a genus of compounds as defined in step (u) (based on structural Formula I), with the proviso that the compound or specie is not a 6-(4-(5-(3-(trifluoromethyl)phenylamino)-4H-1,2,4-triazol-3-yl)phenoxy)-2-(methylthio)pyrimidin-4-amine (also can be designated “KG5”);
(w) a compound having structural Formula II:

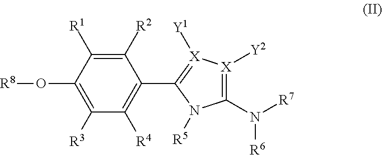

or a pharmaceutically acceptable salt or solvate thereof,

wherein:
X is independently N or C, and wherein when X is N then Y is absent;
R¹-R⁶, Y¹ and Y², are independently selected from the group consisting of hydrogen, deuterium, alkyls, alkenyls, alkynyls, aryls, hetero-alkyls, hetero-alkenyls, hetero-alkynyls, alkoxys, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, thiols, thioethers, thioethers, sulfhydryls, sulfides, disulfides, sulfinyls, sulfos, thiocyanates, isothiocyanates, carbonothioyls, phosphinos, phosphonos, phosphates, silyls, and Si(OH)₃; and
R⁷-R⁸ are independently selected from the group consisting of cycloalkyl, cycloalkenyl, cycloalkynyl, aryl, heterocyle, and extended ring system;
(x) a genus of compounds having a structural Formula II, wherein:
   R⁷ is an aryl having the structure:

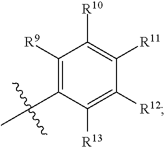

R⁸ is a heterocycle having the structure:

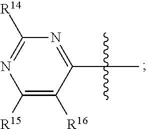

and
R⁹-R¹⁶ are independently selected from the group consisting of hydrogen, deuterium, alkyls, alkenyls, alkynyls, hetero-alkyls, hetero-alkenyls, hetero-alkynyls, alkoxys, halos, hydroxyls, carboxyls, carboxylates, aldehydes, haloformyls, esters, ethers, orthoesters, amines, azides, cyanates, isocyanates, nitrates, nitriles, isonitriles, nitrosos, nitros, nitrosooxy, pyridyls, sulfhydryls, sulfides, disulfides, sulfinyls, thiols, thioethers, sulfos, thiocyanates, isothiocyanates, carbonothioyls, phosphinos, phosphonos, phosphates, silyls, and Si(OH)₃; or
(y) a genus of compounds having a structural Formula II, wherein:
   R¹-R⁶, and R¹⁶ are independently selected from the group consisting of hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, amines, azides, cyanates, nitrates, nitriles, nitros, thiols, and phosphates;
   R⁹-R¹³ are independently selected from the group consisting of hydrogen, deuterium, methyl and CF₃;
   R¹⁴ is selected from the group consisting of (C₁-C₆)alkyls, (C₂-C₆)alkenyls, (C₂-C₆)alkynyls, hetero-(C₁-C₅)alkyls, hetero-(C₁-C₅)alkenyls, hetero-(C₁-C₅)alkynyls, alkoxys, ethers, carboxylic acid, amines, aldehyde, carbonyls, thiols, thioethers, esters, and azides, and
   R¹⁵ is an amine
   In alternative embodiments, R⁷ is an aryl having the structure:

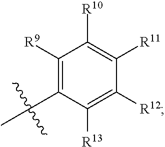

R⁸ is a heterocycle having the structure:

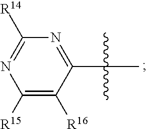

and/or

R⁹-R¹⁶ are independently selected from the group consisting of hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, orthoesters, amines, azides, cyanates, isocyanates, nitrates, nitriles, isonitriles, nitrosos, nitros, nitrosooxy, pyridyls, sulfhydryls, sulfides, disulfides, sulfinyls, thiols, sulfos, thiocyanates, isothiocyanates, carbonothioyls, phosphinos, phosphonos, phosphates, silyls, and Si(OH)₃.

An alternative embodiment comprises the genus of compounds as defined in step (w) (based on structural Formula II) with the proviso that genus does not have (include) the compound or specie 6-(4-(5-(3-(trifluoromethyl)phenylamino)-4H-1,2,4-triazol-3-yl)phenoxy)-2-(methylthio)pyrimidin-4-amine (also can be designated “KG5”).

In alternative embodiments, R¹-R⁶, R⁹-R¹¹, R¹³ and R¹⁶ are independently either hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, amines, azides, cyanates, nitrates, nitriles, nitros, thiols, and phosphates; R¹² is CF₃; R¹⁴ is SCH₃; and/or R¹⁵ is NH₂.

In alternative embodiments, the term “alkyl” refers to an alkyl group that contains 1 to 30 carbon atoms. In alternative embodiments, if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise. In alternative embodiments, the specific substituted alkyl groups include haloalkyl groups, e.g., trihalomethyl groups and trifluoromethyl groups and equivalents.

In alternative embodiments, the term “alkenyl” refers to an alkenyl group that contains 1 to 30 carbon atoms. While a C₁₋alkenyl can form a double bond to a carbon of a parent chain, an alkenyl group of three or more carbons can contain more than one double bond. In alternative embodiments, the alkenyl group will be conjugated, in other cases an alkenyl group will not be conjugated, and yet other cases the alkenyl group may have stretches of conjugation and stretches of nonconjugation. In alternative embodiments, if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkenyl may be substituted or unsubstituted, unless stated otherwise.

In alternative embodiments, the term “alkynyl” refers to an alkynyl group that contains 1 to 30 carbon atoms. While a C₁₋alkynyl can form a triple bond to a carbon of a parent chain, an alkynyl group of three or more carbons can contain more than one triple bond. Where if there is more than 1 carbon, the carbons may be connected in a linear manner, or alternatively if there are more than 3 carbons then the carbons may also be linked in a branched fashion so that the parent chain contains one or more secondary, tertiary, or quaternary carbons. An alkynyl may be substituted or unsubstituted, unless stated otherwise.

In alternative embodiments, the term “cylcloalkyl” refers to an alkyl that contains at least 3 carbon atoms but no more than 12 carbon atoms connected so that it forms a ring. In alternative embodiments, a “cycloalkyl” moiety encompasses from 1 to 7 cycloalkyl rings, wherein when the cycloalkyl is greater than 1 ring, then the cycloalkyl rings are joined so that they are linked, fused, or a combination thereof. In alternative embodiments, the a cycloalkyl may be substituted or unsubstituted, or in the case of more than one cycloalkyl ring, one or more rings may be unsubstituted, one or more rings may be substituted, or a combination thereof A cycloalkyl groups can include bicyclic and tricyclic alkyl groups.

In alternative embodiments, the term “cycloalkenyl” refers to an alkene that contains at least 3 carbon atoms but no more than 12 carbon atoms connected so that it forms a ring. In alternative embodiments, the “cycloalkenyl” moiety encompasses from 1 to 7 cycloalkenyl rings, wherein when the cycloalkenyl is greater than 1 ring, then the cycloalkenyl rings are joined so that they are linked, fused, or a combination thereof. A cycloalkenyl may be substituted or unsubstituted, or in the case of more than one cycloalkenyl ring, one or more rings may be unsubstituted, one or more rings may be substituted, or a combination thereof. A cycloalkenyl groups can include bicyclic and tricyclic alkenyl groups.

In alternative embodiments, the term “cycloalkynyl” refers to an alkyl that contains at least 8 carbon atoms but no more than 12 carbon atoms connected so that it forms a ring. In alternative embodiments, the “cycloalkynyl” moiety encompasses from 1 to 7 cycloalkynyl rings, wherein when the cycloalkynyl is greater than 1 ring, then the cycloalkynyl rings are joined so that they are linked, fused, or a combination thereof. A cycloalkynyl may be substituted or unsubstituted, or in the case of more than one cycloalkynyl ring, one or more rings may be unsubstituted, one or more rings may be substituted, or a combination thereof. A cycloalkynyl groups can include bicyclic and tricyclic alkynyl groups.

In alternative embodiments, the term “aryl” refers to a conjugated planar ring system with delocalized pi electron clouds that contain only carbon as ring atoms. In alternative embodiments, the “aryl” moiety encompasses from 1 to 7 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof.

In alternative embodiments, the term “heterocycle” refers to ring structures that contain at least 1 noncarbon ring atom. A “heterocycle” moiety encompasses encompass from 1 to 7 heterocycle rings wherein when the heterocycle is greater than 1 ring the heterocycle rings are joined so that they are linked, fused, or a combination thereof. A heterocycle may be aromatic or nonaromatic, or in the case of more than one heterocycle ring, one or more rings may be nonaromatic, one or more rings may be aromatic, or a combination thereof. A heterocycle may be substituted or unsubstituted, or in the case of more than one heterocycle ring one or more rings may be unsubstituted, one or more rings may be substituted, or a combination thereof. In alternative embodiments, the noncarbon ring atom is N, O, S, Si, Al, B, or P. In alternative embodiments, where there is more than one noncarbon ring atom, these noncarbon ring atoms can either be the same element, such as N, or combination of different elements, such as N and oxygen (O). In alternative embodiments, examples of heterocycles include, but are not limited to: a monocyclic heterocycle such as, aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide; and polycyclic heterocycles such as, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine. In addition to the polycyclic heterocycles described above, heterocycle includes polycyclic heterocycles wherein the ring fusion between two or more rings includes more than one bond common to both rings and more than two atoms common to both rings. In alternative embodiments, examples of such bridged heterocycles include quinuclidine, diazabicyclo[2.2.1]heptane and 7-oxabicyclo[2.2.1]heptane.

In alternative embodiments, the term “hetero-” when used as a prefix, such as, hetero-alkyl, hetero-alkenyl, hetero-alkynyl, or hetero-hydrocarbon, refers to the specified hydrocarbon group having one or more carbon atoms replaced by non-carbon atoms as part of the parent chain. In alternative embodiments, examples of such noncarbon atoms include, but are not limited to, N, O, S, Si, Al, B, and P. In alternative embodiments, if there is more than one noncarbon atom in the hetero-chain then this atom may be the same element or may be a combination of different elements, such as N and O. Examples of hetero-alkyls which contain S, include but are not limited to, CH₂SCH₃, SCH₃, SCH₂CH₃, SCH(CH₃)₂ and the like.

In alternative embodiments, the term “extended mixed ring system” refers to a group that is comprised of (comprises) at least 2 rings but no more than 7 rings, and wherein at least one ring is selected from a ring group that is different from another ring's group. In alternative embodiments, examples of ring groups include, but are not limited to, cycloalkyl, cycloalkenyl, cycloalkynyl, aryl, and heterocycle. Each ring may be optionally substituted. The rings comprising the mixed extended ring system may be joined so that they are linked, fused, or a combination thereof.

In alternative embodiments, the term “unsubstituted” with respect to hydrocarbons, heterocycles, and the like, refers to structures wherein the specified group contains no substituents.

In alternative embodiments, the term “substituted” with respect to hydrocarbons, heterocycles, and the like, refers to structures wherein the specified group contains one or more substituents.

In alternative embodiments, the term “substituent” refers to an atom or group of atoms substituted in place of a hydrogen atom. In alternative embodiments, the substituent includes deuterium (D) atoms. For example, in alternative embodiments, all of the hydrogens of a molecule used to practice this invention is replaced by a deuterium, or alternatively, only a subset of hydrogen atoms are replaced by D, for example, only the H atoms involved in the allosteric interaction of the composition with c-RAF are deuterated.

As used herein, a wavy line intersecting another line that is connected to an atom indicates that this atom is covalently bonded to another entity that is present but not being depicted in the structure.

The invention provides uses of a compound or composition in the preparation of a medicament for regulating or modulating cell growth or mitosis, angiogenesis, or regulating or modulating phospho-5338 CRAF localization to centrosomes/mitotic spindle poles, comprising administering a composition or compound having a structure as set forth herein, e.g., a composition or compound for practicing a method of the invention. A sufficient amount of the compound or composition can be administered to the cell to regulate or modulate cell growth or mitosis. In alternative embodiments, an allosteric c-RAF activator increases or unregulates cell growth or mitosis, or angiogenesis, or blocks G2M arrest. In alternative embodiments, the use comprises administering an allosteric c-RAF inhibitor, or a composition or an agent which inhibits or prevents localization of a c-RAF to a mitotic spindle, or inhibits, suppresses or decreases cell growth or mitosis, or inhibits or suppresses angiogenesis, or causes cell growth arrest. In alternative embodiments, the use comprises administering an allosteric c-RAF inhibitor inhibits phospho-5338 CRAF localization to centrosomes/mitotic spindle pole. In alternative embodiments the use of the compound or composition, or the medicament, reduces, treats or ameliorates the level of disease in a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

In alternative embodiments the invention provides compounds or compositions for use in regulating or modulating cell growth or mitosis, angiogenesis, or regulating or modulating phospho-S338 CRAF localization to centrosomes/mitotic spindle poles, wherein the compound or composition has a structure as set forth herein, e.g., for practicing a method of the invention. In alternative embodiments, a sufficient amount of the composition is administered to the cell to regulate or modulate cell growth or mitosis. In alternative embodiments, administering an allosteric c-RAF activator increases or unregulates cell growth or mitosis, or angiogenesis, or blocks G2M arrest. In alternative embodiments, administering an allosteric c-RAF inhibitor, or a composition or an agent which inhibits or prevents localization of a c-RAF to a mitotic spindle, or inhibits, suppresses or decreases cell growth or mitosis, or inhibits or suppresses angiogenesis, or causes cell growth arrest. In alternative embodiments, administering an allosteric c-RAF inhibitor inhibits phospho-5338 CRAF localization to centrosomes/mitotic spindle pole. In alternative embodiments, use of the compound, or the medicament, reduces, treats or ameliorates the level of disease in a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments of the invention and are not meant to limit the scope of the invention as encompassed by the claims.

FIG. 1 illustrates a mechanism of tumorigenesis mediated by kinase-dead BRAF in the presence of oncogenic RAS, as described by Heidorn (2010) Cell, January 22; 140(2): 209-221, as further discussed in Example 1, below.

FIG. 2 illustrates that RAF inhibitors induce CRAF p338 and cell activation, as further discussed in Example 1, below.

FIG. 3 illustrates data showing Tamoxifen-only treated patients disease free survival (DFS) curves, as further discussed in Example 1, below.

FIG. 4 illustrates the effect of KG5 on orthotopic MDA-MB-231 breast cancer cells after three days, as further discussed in Example 1, below.

FIG. 5 illustrates the effect of KG5 on orthotopic R40P murine pancreatic tumor cells after three days, as further discussed in Example 1, below.

FIG. 6 illustrates data showing that KG5 inhibits phosphorylation of pS-338 (phosphorylated serine 338) in the activation domain of c-RAF, as further discussed in Example 1, below.

FIG. 7 illustrates data showing that KG5 and KG38 prevent RAF dimerization, and pS-338 (phosphorylated serine 338 RAF) in XPA-1 cells (human pancreatic cancer cells), as further discussed in Example 1, below.

FIG. 8 illustrates the GI₅₀ of KG5 in nM, on various breast cancer cell lines (the NCI renamed the IC₅₀ value, the concentration that causes 50% growth inhibition, the GI50 (or GI₅₀) value to emphasize the correction for the cell count at time zero; thus, GI50 is the concentration of test drug where 100×(T−T0)/(C−T0)=50), as further discussed in Example 1, below.

FIG. 9 illustrates graphically illustrates exemplary compounds that approach picomolar (pM) EC50s in A549 lung cancer cells and T47D breast cancer cells, including KG5, H3-3, H3-7, H3-21 and K38-B, as further discussed in Example 1, below.

FIG. 10 graphically illustrates exemplary compounds that approach picomolar (pM) EC50s in (MDA-MB-231 breast cancer) cell viability assays, including KG5, K1-72, H3-3, H3-7, H3-9, H3-11, H3-21 and K38-B, as further discussed in Example 1, below.

FIG. 11 illustrates an image of cells demonstrating that KG5 disrupts dimerization of RAF in A549 (adenocarcinomic human alveolar basal epithelial) cells, as further discussed in Example 1, below.

FIG. 12 illustrates an image of cells demonstrating that sorafenib and PLX4032 induce proliferation in A549 (adenocarcinomic human alveolar basal epithelial) cells, as further discussed in Example 1, below.

FIG. 13 illustrates an image of cells demonstrating that KG5 disrupts dimerization of RAF in XPA-1 cells (human pancreatic cancer cells), as further discussed in Example 1, below.

FIG. 14 illustrates an image of cells demonstrating that KG5 disrupts dimerization of RAF in XPA-1 cells (human pancreatic cancer cells), as further discussed in Example 1, below.

FIG. 15 schematically illustrates RAF functions other than the canonical MAPK signaling pathway, as further discussed in Example 1, below.

FIG. 16 illustrates an image of cells demonstrating that KG5 suppresses growth of orthotopic pancreatic carcinoma, as further discussed in Example 1, below.

FIG. 17 illustrates an image of cells demonstrating that KG5 suppresses growth of orthotopic pancreatic carcinoma, as further discussed in Example 1, below.

FIG. 18 illustrates an image of cells demonstrating that KG5 disrupts tumor angiogenesis, as further discussed in Example 1, below.

FIG. 19 illustrates an image of cells demonstrating the effect of KG5 on tumor cells, as further discussed in Example 1, below.

FIG. 20 illustrates graphically illustrates that KG5 blocks cells in G2/M, as further discussed in Example 1, below.

FIG. 21 graphically and by image illustrates that KG5 acts like a mitotic poison and arrests cells in G2/M, as further discussed in Example 1, below.

FIG. 22 illustrates an image of cells demonstrating that KG5 blocks prometaphase, as further discussed in Example 1, below.

FIG. 23 illustrates an image of cells demonstrating that c-RAF localizes to mitotic spindles and midbodies, as further discussed in Example 1, below.

FIG. 24 illustrates an image of cells demonstrating that KG5 prevents c-RAF recruitment to the mitotic spindle poles, as further discussed in Example 1, below.

FIG. 25 illustrates an image of cells demonstrating that KG5 and taxol both arrest mitosis, but each with a different mechanism of action, as further discussed in Example 1, below.

FIG. 26 illustrates an image of cells demonstrating that sorafenib does not effect c-RAF localization to mitotic spindles and midbodies, as further discussed in Example 1, below.

FIG. 27 illustrates an image of cells demonstrating that phosphorylated serine 338 c-RAF localizes at mitotic spindles and midbodies, as further discussed in Example 1, below.

FIG. 28 illustrates an image of cells demonstrating that KG5 prevents phosphorylated serine 338 c-RAF recruitment to the mitotic spindle, as further discussed in Example 1, below.

FIG. 29A illustrates an image of phosphorylated serine 338 c-RAF staining in human glioblastoma biopsy cells; FIG. 29B illustrates an image of human glioblastoma biopsy cells in telophase, where phosphorylated serine 338 c-RAF stains at the midbody;

FIG. 29C illustrates an image of human glioblastoma biopsy cells in metaphase where phosphorylated serine 338 c-RAF stains at the mitotic spindle, as further discussed in Example 1, below.

FIG. 30 illustrates an image of breast orthotopic xenograft cells demonstrating that phosphorylated serine 338 c-RAF localizes at mitotic spindle poles, as further discussed in Example 1, below.

FIG. 31 illustrates an image of breast orthotopic xenograft cells demonstrating that phosphorylated serine 338 c-RAF localizes at mitotic spindle poles, as further discussed in Example 1, below.

FIG. 32A schematically illustrates an exemplary protocol for the synchronization of mouse embryonic fibroblasts with nocodazole; FIG. 32B graphically illustrates data demonstrating that cells lacking c-RAF are delayed in mitosis, as further discussed in Example 1, below.

FIG. 33 graphically illustrates data demonstrating that serine 338 c-RAF is required for mitosis, as further discussed in Example 1, below.

FIG. 34: FIG. 34(a) graphically illustrates a cell cycle analysis of WT and CRAF−/− MEFs; FIG. 34(b) graphically illustrates how WT and CRAF−/− MEFs were synchronized at pro-metaphase with a thymidine-nocodazole block; FIG. 34(c) illustrates confocal microscopy images of WT and CRAF−/− cells progressing through mitosis at 0, 60 and 360 minutes after release from pro-metaphase blockade; FIG. 34(d) graphically illustrates cell cycle analysis data where WT and CRAF−/− MEFs were transfected with vector control, WT CRAF, kinase dead (K375M) CRAF, phospho-mimetic (S338D) CRAF or non-phosphorylatable (S338A) CRAF mutants; and, FIG. 34(e) illustrates an image of immunohistochemical staining of phospho-S338 CRAF and phospho-histone H3 (mitotic marker) in orthotopic breast and tumor xenografts untreated or treated systemically with KG5, and graphically summarizes data from this staining; as described in detail in Example 2, below.

FIG. 35: FIG. 35(a) illustrates an immunoblot analysis of human colon carcinoma HCT-116 cells asynchronized and synchronized at pro-metaphase; FIG. 35(b) illustrates confocal microscopy images of human pancreatic XPA-1 and glioblastoma U251 cells during mitosis, stained for phospho-5338 CRAF, α-tubulin and DNA; FIG. 35(c) illustrates an immunoblot analysis of γ-tubulin immunoprecipitates from human colon carcinoma HCT-116 cells asynchronized and synchronized at pro-metaphase; FIG. 35(d) illustrates an immunohistochemical staining of phospho-5338 CRAF in tumor biopsies from breast cancer patients; FIG. 35(e) illustrates confocal microscopy images of XPA-1 cells treated with KG5, sorafenib, ZM336372, L779450 or paclitaxel and stained for CRAF, γ-tubulin and DNA; as described in detail in Example 2, below.

FIG. 36: FIG. 36(a) illustrates an immunoblot analysis of CRAF immunoprecipitates from HCT-116 asynchronized and synchronized at pro-metaphase cells; FIG. 36(b) illustrates confocal microscopy images of HCT-116 cells synchronized at G2 and pro-metaphase and stained for CRAF, phospho-T210 Plk1 and DNA; FIG. 36(c) illustrates an immunoblot analysis of Plk1 immunoprecipitates from WT, CRAF−/− and BRAF−/− MEFs; FIG. 36(d) illustrates an immunoblot analysis from G1-M, of phospho-S338 CRAF, total CRAF, phospho-T210 Plk1, total Plk1, cyclin B and tubulin of HCT-116 cells synchronized at the G1/S boundary; FIG. 36(e) illustrates an immunoblot analysis of asynchronous and mitotic WT and CRAF−/− MEFs; FIG. 36(f) illustrates confocal microscopy images of WT and CRAF−/− MEFs at pro-metaphase, cells were stained for a tubulin, phospho-T210 Plk1 and DNA; as described in detail in Example 2, below.

FIG. 37: FIG. 37(a) illustrates images of HCT-116 human colon carcinoma cells ectopically expressing either WT RAF or S338D mutant CRAF arrested in pro-metaphase and subsequently allowed to progress through mitosis; FIG. 37(b) graphically illustrates Plk1 kinase activity assay performed in HCT-116 cells expressing WT CRAF or a phospho-mimetic S338D CRAF mutant; FIG. 37(c) illustrates HCT-116 cells expressing WT or S338D CRAF Flag tagged injected subcutaneously in the flank of immune-compromised nude mice, results are graphically illustrated; FIG. 37(d) illustrates immunohistochemical staining of phospho-Plk1 and phospho-MEK in mouse tissues from tumors expressing WT or S338D CRAF; FIG. 37(e) illustrates immunoblot analysis of phospho-T210 Plk1, Plk1, phospho-MEK, MEK and Flag from tumor lysates from HCT-116 cells expressing WT or S338D CRAF; as described in detail in Example 2, below.

FIG. 38: FIG. 38(a) illustrates bright field images, graphically illustrates cell cycle analysis and illustrates confocal microscopy images of XPA-1 cells treated for 20 hours with the allosteric RAF inhibitor KG5, the ATP-competitive inhibitor Sorafenib, the MEK inhibitor PD0325901; FIG. 38(b) illustrates representative confocal microscopy images of untreated XPA-1 tumor cells undergoing mitosis (left image) and XPA-1 cell arrested with KG5 at pro-metaphase (right image); FIG. 38(c) graphically illustrates data from XPA-1 cells transfected with a vector control or active MEK treated with 1 μM KG5 for 20 hours or left untreated, and the G2/M population was quantified by flow cytometry; an immunoblot of this study is also illustrated (inset); FIG. 38(d) illustrates immunoblot analysis of XPA-1 cells untreated or treated with 1 μM KG5 for 18 hours; FIG. 38(e) graphically illustrates data from studies where HCT-116 cells stably expressing WT or the phospho-mimetic S338D CRAF construct were treated with 1 μM KG5 for 18 hours, where cells arrested in G2/M were quantified by flow cytometry; an immunoblot of this study is also illustrated (inset); as described in detail in Example 2, below.

FIG. 39 graphically illustrates cell cycle analysis of XPA-1 cells treated with the allosteric RAF inhibitor KG5, the microtubule stabilizer paclitaxel, or the ATP-competitive RAF inhibitors sorafenib, L-779450, GW 5074, and ZM-336372; cells were harvested after 6, 18, 24, 48 and 72 hours and the percentages of cells in G2/M and SubG1 were determined by flow cytometry; as described in detail in Example 2, below.

FIG. 40: FIG. 40(a) graphically illustrates cell cycle analysis and G2/M quantification of human colon (HCT-116), pancreatic (Mia-Paca2, FG, XPA-1, BXPC3), breast (MDA-MB-231) and brain (U251) cancer cell lines untreated or treated for 20 hours with 5 μM of KG5; FIG. 40(b) graphically illustrates quantification of cells arrested in G2/M after HCT-116 cells were cultured under low (30%) or high (90%) confluency and treated with 1 μM KG5 for 20 hours; FIG. 40(c) graphically illustrates HCT-116 and XPA-1 cells arrested in pro-metaphase after they were treated for 20 hours with 1 μM of KG5, stained for α-tubulin/DNA, and cells arrested in pro-metaphase were quantified by confocal microscopy; Lysates from the same cells were also analyzed by immunoblotting for the expression of the mitotic marker phospho-Histone H3 (inset); as described in detail in Example 2, below.

FIG. 41: FIG. 41(a) illustrates immunohistochemical staining of phospho-S338 CRAF in normal human brain and human glioblastoma tissues; FIG. 41(b) illustrates immunohistochemical staining of phospho-S338 CRAF in orthotopic brain tumor xenografts untreated or treated systemically with KG5 (50 mg/Kg) for 3 days; as described in detail in Example 2, below.

FIG. 42: FIG. 42(a) graphically illustrates quantification of WT and CRAF−/− cells in the G1, S and G2/M phases of the cell cycle; FIG. 42(b) illustrates an immunoblot analysis of CRAF−/− MEFs transfected with WT, kinase dead (K375M), phospho-mimetic (S338D) and non-phosphorylatable (S338A) FLAG tagged CRAF constructs; FIG. 42(c) graphically illustrates cell cycle analysis of CRAF−/− MEFs transfected with vector control or S338D CRAF; FIG. 42(d) graphically illustrates cells at pro-metaphase quantified using confocal microscopy; FIG. 42(e) graphically illustrates HCT-116 cells were transfected with control or CRAF siRNAs and cells in G2/M were quantified by flow cytometry; Lysates of these cells were analyzed for CRAF expression by immunoblotting (inset); FIG. 42(f) graphically illustrates XPA-1 cells ectopically expressing either WT RAF or S338A mutant CRAF arrested in pro-metaphase; FIG. 42(g) illustrates confocal microscopy images of cells progressing through mitosis at 0, 20, 100 and 120 minutes after release from pro-metaphase blockade; cells were stained for a tubulin and DNA; as described in detail in Example 2, below.

FIG. 43 illustrates confocal microscopy images of human HCT-116 cells synchronized at the G1/S boundary by a double thymidine block and stained for γ tubulin, phospho-5338 CRAF and DNA; white arrows indicate localization of phospho-S338 CRAF at the centrosomes in G2 and the mitotic spindle poles from pro-metaphase to anaphase; as described in detail in Example 2, below.

FIG. 44: FIG. 44(a) illustrates confocal microscopy images of WT, CRAF−/− and BRAF−/− MEFs at prophase and metaphase stained for phospho-5338 CRAF, γ-tubulin and DNA; white arrows indicate localization of phospho-S338 CRAF at the mitotic spindle poles; FIG. 44(b) illustrates immunohistochemical staining of phospho-S338 CRAF in tumor biopsies from mouse orthotopic breast tumour xenografts; as described in detail in Example 2, below.

FIG. 45: FIG. 45(a) illustrates immunoblot analysis of Flag immunoprecipitates from XPA-1 cells expressing Flag-tagged WT or S338A CRAF; FIG. 45(b) illustrates confocal microscopy images of a HCT-116 synchronized in G2 or mitosis and stained for phospho-5338 CRAF, phospho-T288 Aurora A and DNA; white arrows indicate colocalization between phospho-5338 CRAF and phospho-T288 Aurora A; FIG. 45(c) illustrates immunoblot analysis of CRAF immunoprecipitates from XPA-1 cells asynchronized or synchronized in mitosis; FIG. 45(d) illustrates immunoblot analysis of Flag immunoprecipitates from XPA-1 cells expressing Flag-tagged WT or kinase dead (K375M) CRAF; FIG. 45(e) illustrates immunoblot analysis of BRAF immunoprecipitates from HCT-116 cells asynchronized or synchronized in mitosis; as described in detail in Example 2, below.

FIG. 46: FIG. 46(a) illustrates Plk1 kinase activity assay performed in WT and CRAF−/− MEFs; FIG. 46(b) illustrates immunoblot analysis of XPA-1 cells asynchronous or arrested in mitosis by treatment with KG5, nocodazole or paclitaxel; FIG. 46(c) illustrates Plk1 kinase activity assay performed in HCT-116 cells treated with nocodazole or nocodazole and KG5; FIG. 46(d) illustrates immunoblot analysis of CenpF immunoprecipitates from HCT-116 cells untreated or treated with KG5; FIG. 46(e) illustrates confocal microscopy images of XPA-1 cell at prophase (upper panel), pro-metaphase control untreated (middle panel) or treated with KG5 (lower panel); cells were stained for phospho-T210 Plk1, a tubulin and DNA; thick white arrows indicate localization of pPlk1 at the mitotic spindle pole and narrow white arrows indicate localization of pPlk1 at the kinetochores; as described in detail in Example 2, below.

FIG. 47 illustrates cell staining and data showing that allosteric inhibition or depletion of CRAF prevents active Plk1 accumulation at the kinetochores in pro-metaphase; the table indicates: Plk1 localization before mitosis and during the different stages of mitosis (from prophase to telophase); as described in detail in Example 2, below.

FIG. 48: FIG. 48(a) graphically illustrates data from studies where CRAF−/− MEFs were transfected with vector control, kinase dead (K375M) CRAF or a constitutively active T210D Plk1 mutant and the G2/M population was quantified by flow cytometry; FIG. 48(b) illustrates an immunoblot analysis of CRAF−/− MEFs transfected with kinase dead (K375M) CRAF FLAG tagged or a constitutively active T210D Plk1 HA-tagged mutant; as described in detail in Example 2, below.

FIG. 49 illustrates staining of RFP-labeled U-87 human glioblastoma cells expressing WT or S338D CRAF, which were orthotopically implanted in the brain of immune-compromised nude mice; and a graphic illustration summarizing the results; as described in detail in Example 2, below.

FIG. 50: FIG. 50(a) illustrates immunoblot analysis of phospho-T210 Plk1, Plk1, phospho-MEK, MEK, Flag and tubulin from U-87 cells expressing WT or S338D/K375M CRAF; FIG. 50(b) graphically illustrates data from studies where U-87 cells expressing WT or S338D/K375M CRAF were injected subcutaneously in the flank of immune-compromised nude mice; tumor size measurements are shown; as described in detail in Example 2, below.

FIG. 51 illustrates a model depicting the MEK-independent role of CRAF in mitosis and tumor progression; as described in detail in Example 2, below.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, the invention provides methods for ameliorating, preventing and/or treating diseases, infections and/or conditions having unwanted, pathological or aberrant cell proliferation, or that are responsive to inhibition or arrest of cell growth, by administration of an allosteric c-RAF inhibitor and/or any agent which prevents localization of c-RAF to mitotic spindles. The inventors discovered that allosteric c-RAF inhibitors, but not ATP-competitive c-RAF inhibitors, block proliferating cells in mitosis by preventing c-RAF localization to the mitotic spindles.

We identified a MEK-independent role for RAF in tumor growth. Specifically, in mitotic cells, c-RAF (or CRAF) becomes highly phosphorylated on serine 338 and localizes to the mitotic spindles of proliferating tumor cells in vitro and in orthotopic mouse model xenografts or biopsies from cancer patients. Treatment of tumors with allosteric, but not ATP-competitive RAF inhibitors prevents c-RAF (or CRAF) phosphorylation on serine 338, localization to the mitotic spindle and causes cell cycle arrest at pro-metaphase. Furthermore, we identify phospho-5338 c-RAF (or CRAF) as a potential biomarker for tumor progression and a surrogate marker for allosteric RAF blockade. Mechanistically, c-RAF (or CRAF), but not BRAF is found in a complex with Aurora-A and Polo-like kinase 1 at the centrosomes/spindle poles during G2/Mitosis. Indeed, allosteric or genetic inhibition of phospho-S338 c-RAF (or CRAF) impairs Plk1 activity and its accumulation at kinetochores causing pro-metaphase arrest, while a phospho-mimetic S338D c-RAF (or CRAF) mutant potentiates Plk1 activation, mitosis and tumor progression in mice. These findings reveal a previously undefined role for RAF in tumor progression beyond the RAF-MEK-ERK paradigm, opening new avenues for targeting RAF, particularly c-RAF, in oncology.

While the invention is not limited by any particular mechanism of action, the invention provides methods for ameliorating, preventing and/or treating diseases, infections and/or conditions having unwanted, pathological or aberrant cell proliferation, or that are responsive to inhibition or arrest of cell growth, by promoting G2M arrest by blocking association of c-RAF with the mitotic spindle and/or mid-body during mitosis, through (1) allosteric inhibition of c-RAF, or by (2) delivering any agent which prevents localization of c-RAF to mitotic spindles (the mid-body is a transient structure in mammalian cells that is present near the end of cytokinesis just prior to the complete separation of the dividing cell; the midbody structure contains bundles of microtubules derived from the mitotic spindle which compacts during the final stages of cell division).

In alternative embodiments, the invention provides methods to determine which patients would benefit from allosteric c-RAF inhibitors by detection of Serine-338 phosphorylated c-RAF localized to the mitotic spindle, e.g., as seen or detected in pathology slides. Another aspect of the invention comprises determining therapeutic efficacy of an agent that blocks cell proliferation at the mitotic phase by detection of Serine-338 phosphorylated c-RAF localized to the mitotic spindle, e.g., in pathology slides.

In alternative embodiments, the invention provides methods of administering allosteric c-RAF inhibitors that are safer, more effective than ATP-competitive RAF inhibitors. In alternative embodiments, the invention provides methods of administering allosteric c-RAF inhibitors to treat, prevent, reverse or ameliorate a cancer, inflammatory diseases, and other diseases, infections or conditions of responsive decreasing, inhibiting or diminishing unwanted or abnormal cell proliferation.

In alternative embodiments, the invention provides methods for in vitro and in vivo administration of a c-RAF modulator, e.g., to inhibit or to promote mitosis.

In alternative embodiments, the invention provides methods using c-RAF inhibitors comprising the compound 6-(4-(5-(3-(trifluoromethyl)phenylamino)-4H-1,2,4-triazol-3-yl)phenoxy)-2-(methylthio)pyrimidin-4-amine (designated “KG5”) and KG38.

In alternative embodiments, compounds used to practice the invention, including e.g., KG5, are described in detail in Murphy et al. (Mar. 2, 2010) vol. 107:4299-4304.

In alternative embodiments, the invention provides methods using c-RAF inhibitors comprising: pyrazolo[1,5-a]pyrimidine scaffold as B-Raf kinase inhibitors, see e.g., Gopalsamy, et al. Bioorg Med Chem. Lett. 2009 Dec. 15; 19(24):6890-2. Epub 2009 Oct. 22; or pyrazolo[1,5-a]pyrimidine-3-carboxylates; see e.g., Gopalsamy, et al. Bioorg Med Chem. Lett. 2009 May 15; 19(10):2735-8. Epub 2009 Mar. 28; or compounds where a scaffold pyrazolo[1,5-a]pyrimidine is replaced with different thienopyrimidine and thienopyridine scaffolds, e.g., as described in Gopalsamy, et al. Bioorg Med Chem. Lett. 2010 Apr. 15; 20(8):2431-4. Epub 2010 Mar. 9; or comprising use of non-hinge-binding pyrazolo[1,5-a]pyrimidines as potent B-Raf kinase inhibitors, e.g., see Berger, et al. Bioorg Med Chem. Lett. 2009 Dec. 1; 19(23):6519-23. Epub 2009 Oct. 26, who prepared a series of C-3 substituted N-(3-(pyrazolo[1,5-a]pyrimidin-7-yl)phenyl)-3-(trifluoromethyl)benzamides, and X-ray crystallography studies revealed that one of the more potent inhibitors bound to B-Raf kinase without forming a hinge-binding hydrogen bond.

In alternative embodiments, the invention provides diagnostic methods comprising the detection of p-ser338 c-RAF staining at a mitotic spindle in a cell, e.g., on a pathology slide, or equivalent histologic visualization apparatus. The invention have detected p-ser338 C-RAF staining at a mitotic spindle in a human glioblastoma biopsy pathology slide.

The inventors have found that allosteric c-RAF inhibitors but not ATP-mimetic RAF inhibitors strongly decrease cell proliferation. Allosteric c-RAF inhibitors block proliferating cells in mitosis (phase of the cell cycle in which the cell divides in two). While the invention is not limited by any particular mechanism of action, in alternative embodiments, the invention provides methods comprising use of allosteric c-RAF inhibitors to arrest cells in mitosis by preventing Raf localization to mitotic spindles, which is the cellular structure required for proper chromosome segregation and cell division. c-RAF localization to the mitotic spindles is a new and surprising finding and suggests that c-RAF may help cells go through mitosis. In alternative embodiments, any pharmacological agent that directly or indirectly will prevent translocation of c-RAF to the mitotic spindle will arrest cells in mitosis and thus block cell proliferation can be used. This is a completely new mechanism of action for a c-RAF inhibitor that makes allosteric c-RAF inhibitors great therapeutically effective compounds for the treatment of diseases characterized by an excessive cell proliferation including all types of cancer and inflammatory diseases.

In alternative embodiments, the invention uses allosteric c-Raf inhibitors that are very selective drugs with very low toxicity. While the invention is not limited by any particular mechanism of action, this mechanism of action of allosteric c-RAF inhibitors is very unique since they arrest proliferating cells in mitosis by preventing c-Raf localization to mitotic spindles. This is a new mechanism of action that has never been described for c-RAF inhibitors and makes allosteric c-RAF inhibitors great anti-proliferative therapeutically effective compounds.

We have found that allosteric c-RAF inhibitors (type II inhibitors), can circumvent the limitations of the type I inhibitors by blocking cell proliferation at the mitotic phase.

In alternative embodiments, the invention provides methods for decreasing or blocking cell proliferation with allosteric c-Raf inhibitors to ameliorate or reduce the level of conditions and diseases such as: all types of cancer, all inflammatory diseases and diseases, conditions (e.g., allergies) and infections having an inflammatory component, such as e.g., multiple sclerosis, rheumatoid arthritis, inflammatory bowel disease, ulcerative colitis or Crohn's disease.

### Pharmaceutical Compositions

In alternative embodiments, the invention provides pharmaceutical compositions for practicing the methods of the invention, e.g., pharmaceutical compositions for ameliorating, preventing and/or treating diseases, infections and/or conditions having unwanted, pathological or aberrant cell proliferation, or that are responsive to inhibition or arrest of cell growth, by administration of an allosteric c-RAF inhibitor and/or any agent which prevents localization of c-RAF to mitotic spindles. The invention provides compositions as described herein, including pharmaceutical compositions, e.g., in the manufacture of medicaments for ameliorating, preventing and/or treating diseases, infections and/or conditions having unwanted, pathological or aberrant cell proliferation.

In alternative embodiments, compositions used to practice the methods of the invention are formulated with a pharmaceutically acceptable carrier. In alternative embodiments, the pharmaceutical compositions used to practice the methods of the invention can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Therapeutic agents used to practice the methods of the invention can be administered alone or as a component of a pharmaceutical formulation (composition).

The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions used to practice the methods of the invention include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Pharmaceutical formulations used to practice the methods of the invention can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, geltabs, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations used to practice the methods of the invention can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., a composition used to practice the methods of the invention) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration hydrophobic active agents used to practice the methods of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In practicing this invention, the pharmaceutical compounds can also be administered by in intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In practicing this invention, the pharmaceutical compounds can be delivered by transdermally, by a topical route, formulated as applicator sticks, solutions, suspensions, emulsions, gels, creams, ointments, pastes, jellies, paints, powders, and aerosols.

In practicing this invention, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In practicing this invention, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations used to practice the methods of the invention can be lyophilized. The invention provides a stable lyophilized formulation comprising a composition of the invention, which can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations used to practice the methods of the invention can be delivered by the use of liposomes (see also discussion, below). By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The formulations used to practice the methods of the invention can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). For example, in alternative embodiments, pharmaceutical compositions of the invention are administered in an amount sufficient to treat, prevent and/or ameliorate normal, dysfunction (e.g., abnormally proliferating) cell, e.g., cancer cell, or blood vessel cell, including endothelial and/or capillary cell growth; including neovasculature related to (within, providing a blood supply to) hyperplastic tissue, a granuloma or a tumor. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, an exemplary pharmaceutical formulation for oral administration of compositions used to practice the methods of the invention can be in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods of the invention can further comprise co-administration with other drugs or pharmaceuticals, e.g., compositions for treating cancer, septic shock, infection, fever, pain and related symptoms or conditions. For example, the methods and/or compositions and formulations of the invention can be co-formulated with and/or co-administered with antibiotics (e.g., antibacterial or bacteriostatic peptides or proteins), particularly those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

### Nanoparticles and Liposomes

The invention also provides nanoparticles and liposomal membranes comprising compounds used to practice the methods of the invention which target specific molecules, including biologic molecules, such as polypeptide, including cell surface polypeptides, e.g., polypeptides on abnormally growing cells, cancer cells, cancer stem cells, blood vessel and angiogenic cells. Thus, in alternative embodiments, the invention provides nanoparticles and liposomal membranes targeting diseased and/or tumor (cancer) stem cells and dysfunctional stem cells, and angiogenic cells.

In alternative embodiments, the invention provides nanoparticles and liposomal membranes comprising (in addition to comprising compounds used to practice the methods of the invention) molecules, e.g., peptides or antibodies, that selectively target abnormally growing, diseased, infected, dysfunctional and/or cancer (tumor) cell receptors. In alternative embodiments, the invention provides nanoparticles and liposomal membranes using IL-11 receptor and/or the GRP78 receptor to targeted receptors on cells, e.g., on tumor cells, e.g., on prostate or ovarian cancer cells. See, e.g., U.S. patent application publication no. 20060239968.

In one aspect, the compositions used to practice the methods of the invention are specifically targeted for inhibiting, ameliorating and/or preventing endothelial cell migration and for inhibiting angiogenesis, e.g., tumor-associated or disease- or infection-associated neovasculature.

The invention also provides nanocells to allow the sequential delivery of two different therapeutic agents with different modes of action or different pharmacokinetics, at least one of which comprises a composition used to practice the methods of the invention. A nanocell is formed by encapsulating a nanocore with a first agent inside a lipid vesicle containing a second agent; see, e.g., Sengupta, et al., U.S. Pat. Pub. No. 20050266067. The agent in the outer lipid compartment is released first and may exert its effect before the agent in the nanocore is released. The nanocell delivery system may be formulated in any pharmaceutical composition for delivery to patients suffering from a diseases or condition as described herein, e.g., such as a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

In treating cancer, a traditional antineoplastic agent is contained in the outer lipid vesicle of the nanocell, and an antiangiogenic agent of this invention is loaded into the nanocore. This arrangement allows the antineoplastic agent to be released first and delivered to the tumor before the tumor's blood supply is cut off by the composition of this invention.

The invention also provides multilayered liposomes comprising compounds used to practice this invention, e.g., for transdermal absorption, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition of this invention.

A multilayered liposome used to practice the invention may further include an antiseptic, an antioxidant, a stabilizer, a thickener, and the like to improve stability. Synthetic and natural antiseptics can be used, e.g., in an amount of 0.01% to 20%. Antioxidants can be used, e.g., BHT, erysorbate, tocopherol, astaxanthin, vegetable flavonoid, and derivatives thereof, or a plant-derived antioxidizing substance. A stabilizer can be used to stabilize liposome structure, e.g., polyols and sugars. Exemplary polyols include butylene glycol, polyethylene glycol, propylene glycol, dipropylene glycol and ethyl carbitol; examples of sugars are trehalose, sucrose, mannitol, sorbitol and chitosan, or a monosaccharides or an oligosaccharides, or a high molecular weight starch. A thickener can be used for improving the dispersion stability of constructed liposomes in water, e.g., a natural thickener or an acrylamide, or a synthetic polymeric thickener. Exemplary thickeners include natural polymers, such as acacia gum, xanthan gum, gellan gum, locust bean gum and starch, cellulose derivatives, such as hydroxy ethylcellulose, hydroxypropyl cellulose and carboxymethyl cellulose, synthetic polymers, such as polyacrylic acid, poly-acrylamide or polyvinylpyrollidone and polyvinylalcohol, and copolymers thereof or cross-linked materials.

Liposomes can be made using any method, e.g., as described in Park, et al., U.S. Pat. Pub. No. 20070042031, including method of producing a liposome by encapsulating a therapeutic product comprising providing an aqueous solution in a first reservoir; providing an organic lipid solution in a second reservoir, wherein one of the aqueous solution and the organic lipid solution includes a therapeutic product; mixing the aqueous solution with said organic lipid solution in a first mixing region to produce a liposome solution, wherein the organic lipid solution mixes with said aqueous solution so as to substantially instantaneously produce a liposome encapsulating the therapeutic product; and immediately thereafter mixing the liposome solution with a buffer solution to produce a diluted liposome solution.

The invention also provides nanoparticles comprising compounds used to practice this invention to deliver a composition of the invention as a drug-containing nanoparticles (e.g., a secondary nanoparticle), as described, e.g., in U.S. Pat. Pub. No. 20070077286. In one embodiment, the invention provides nanoparticles comprising a fat-soluble drug of this invention or a fat-solubilized water-soluble drug to act with a bivalent or trivalent metal salt.

Liposomes

The compositions and formulations used to practice the invention can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. For example, in one embodiment, compositions and formulations used to practice the invention are delivered by the use of liposomes having rigid lipids having head groups and hydrophobic tails, e.g., as using a polyethyleneglycol-linked lipid having a side chain matching at least a portion the lipid, as described e.g., in US Pat App Pub No. 20080089928. In another embodiment, compositions and formulations used to practice the invention are delivered by the use of amphoteric liposomes comprising a mixture of lipids, e.g., a mixture comprising a cationic amphiphile, an anionic amphiphile and/or neutral amphiphiles, as described e.g., in US Pat App Pub No. 20080088046, or 20080031937. In another embodiment, compositions and formulations used to practice the invention are delivered by the use of liposomes comprising a polyalkylene glycol moiety bonded through a thioether group and an antibody also bonded through a thioether group to the liposome, as described e.g., in US Pat App Pub No. 20080014255. In another embodiment, compositions and formulations used to practice the invention are delivered by the use of liposomes comprising glycerides, glycerophospholipides, glycerophosphinolipids, glycerophosphonolipids, sulfolipids, sphingolipids, phospholipids, isoprenolides, steroids, stearines, sterols and/or carbohydrate containing lipids, as described e.g., in US Pat App Pub No. 20070148220.

Therapeutically Effective Amount and Dose

In alternative embodiment, pharmaceutical compositions and formulations used to practice the invention can be administered for prophylactic and/or therapeutic treatments; for example, the invention provides methods for treating, preventing or ameliorating: a disease or condition associated with dysfunctional stem cells or cancer stem cells, a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease. In therapeutic applications, compositions are administered to a subject already suffering from a condition, infection or disease in an amount sufficient to cure, alleviate or partially arrest the clinical manifestations of the condition, infection or disease (e.g., disease or condition associated with dysfunctional stem cells or cancer stem cells) and its complications (a “therapeutically effective amount”). In the methods of the invention, a pharmaceutical composition is administered in an amount sufficient to treat (e.g., ameliorate) or prevent a disease or condition associated with dysfunctional stem cells or cancer stem cells. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

### Kits and Instructions

The invention provides kits comprising compositions for practicing the methods of the invention, including instructions for use thereof. In alternative embodiments, the invention provides kits comprising an allosteric c-Raf regulator, e.g., an allosteric c-RAF inhibitors or activator. In alternative embodiments, the invention provides kits comprising a composition, product of manufacture, or mixture or culture of cells for practicing a method of the invention; wherein optionally the kit further comprises instructions for practicing a method of the invention.

The invention will be further described with reference to the following examples; however, it is to be understood that the invention is not limited to such examples.

### EXAMPLES

### Example 1

### Methods of the Invention are Effective for Inhibiting and/or Promoting Cell Growth and Arresting Mitosis

The data presented herein demonstrates methods of the invention are effective for arresting mitosis and/or inhibiting or decreasing cell growth.

In one embodiment, the compound 2-(methylsulfanyl)-6-[4-(2-{[3-(trifluoromethyl)phenyl]amino}-1H-imidazol-5-yl)phenoxy]pyrimidin-4-amine, also designated “KG5”, or “compound 6”, was used to practice this invention. KG5 does not compete for ATP but inhibits phospho-5338 CRAF. As described in Murphy et al. (Mar. 2, 2010) vol. 107:4299-4304, compound 6 (KG5) inhibits c-RAF and PDGFR signaling but does not influence a variety of other cellular targets. Compound 6 directly impacts PDGFRβ autophosphorylation in response to PDGF-BB and inhibits MAPK signaling in response to bFGF (beta-FGF) or VEGF. Lysates of bFGF- or VEGF-stimulated endothelial cells (ECs) treated in the presence or absence of KG5 were analyzed for the activation of a variety of signaling cascades. Whereas KG5 blocked p-MEK and p-ERK in response to either growth factor, it did not suppress p-Aid, suggesting that 6 inhibits RAF because it impacts the MAPK pathway downstream of Ras but upstream of MEK. We also observed no effect on integrin-mediated signaling to FAK or Src and saw no impact on PKC. Importantly, KG5 inhibited bFGF- or VEGF-mediated phosphorylation of MEK on S217/S221 but not on S298. MEK S217/S221 is the well-known RAF phosphorylation site important for MEK activation, whereas MEK S298 has been shown to be phosphorylated by p21-activated kinase (PAK), which can lead to MEK activation. Therefore, KG5 does not directly inhibit PAK activity.

In alternative embodiments, compounds used to practice the invention, their structures, and methods for making them, including e.g., KG5, are described in detail in Murphy et al. (Mar. 2, 2010) vol. 107:4299-4304, and include (comprise or consist of) the following compounds, and equivalents thereof:
N-(3-(4-(pyridin-3-yl)pyrimidin-2-ylamino)-4-methylphenyl)-4-((4-methylpiperazin-1-yl)methyl)benzamide;
1-(4-(2-(methylcarbamoyl)pyridin-4-yloxy)phenyl)-3-(4-chloro-3-(trifluoromethyl)phenyl)urea;
6-(4-(5-(3-(trifluoromethyl)phenylamino)-4H-1,2,4-triazol-3-yl)phenoxy)pyrimidin-4-amine;
6-(4-(5-(3-(trifluoromethyl)phenylamino)-4H-1,2,4-triazol-3-yl)phenoxy)pyrimidine-2,4-diamine;
6-(4-(5-(4-chloro-3-(trifluoromethyl)phenylamino)-4H-1,2,4-triazol-3-yl)phenoxy)pyrimidine-2,4-diamine;
6-(4-(5-(3-(trifluoromethyl)phenylamino)-4H-1,2,4-triazol-3-yl)phenoxy)-2-(methylthio)pyrimidin-4-amine;
5-(4-(2-(methylthio)pyrimidin-4-yloxy)phenyl)-N-(3-(trifluoromethyl)phenyl)-4H-1,2,4-triazol-3-amine;
6-(4-(5-(4-chloro-3-(trifluoromethyl)phenylamino)-4H-1,2,4-triazol-3-yl)phenoxy)-2-(methylthio)pyrimidin-4-amine;
4-(4-(5-(3-(trifluoromethyl)phenylamino)-4H-1,2,4-triazol-3-yl)phenoxy)-6-methoxypyrimidin-2-amine;
6-(4-(5-(3-(trifluoromethyl)phenylamino)-1,3,4-oxadiazol-2-yl)phenoxy)pyrimidine-2,4-Diamine;
6-(4-(5-(3-(trifluoromethyl)phenylamino)-1,3,4-oxadiazol-2-yl)phenoxy)-2-(methylthio)pyrimidin-4-amine;
(Z)-3-((3,5-dimethyl-1 Hpyrrol-2-yl)methylene)indolin-2-one; and/or 3-(3,5-dibromo-4-hydroxybenzylidene)-5-iodoindolin-2-one.

In alternative embodiments, compounds used to practice the invention, and equivalents thereof, comprise or consist of:

2-(methylsulfanyl)-6-[4-(2-{[3-(trifluoromethyl)phenyl]amino}-1H-imidazol-5-yl)phenoxy]pyrimidin-4-amine (also called “KG5”), or 2-(methylsulfanyl)-6-[4-(2-{[3-(trifluoromethyl)phenyl]amino}-1H-imidazol-5-yl)phenoxy]pyrimidin-4-amine (also called “K38-B, or KG38”), having the structure:

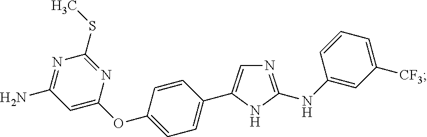 [SMILES: CSc1nc(N)cc(Oc2ccc(-c3cnc(Nc4cccc(C(F)(F)F)c4)[nH]3)cc2)n1]

6-(4-{5-[2,3-dimethylphenyl)amino]-4H-1,2,4-triazol-3-yl}phenoxy)-2-(methylsulfanyl)pyrimidin-4-amine (also called “H3-11”), having the structure:

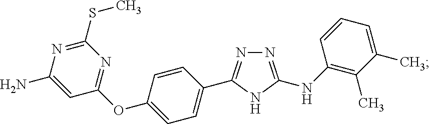 [SMILES: CSc1nc(N)cc(Oc2ccc(-c3nnc(Nc4cccc(C)c4C)[nH]3)cc2)n1]

2-(methylsulfanyl)-6-{4-[5-(5,6,7,8-tetrahydronaphthalen-1-ylamino)-4H-1,2,4-triazol-3-yl]phenoxy}pyrimidin-4-amine (also called “H3-21”), having the structure:

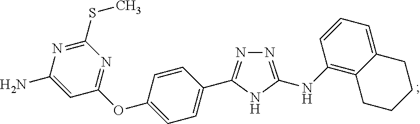 [SMILES: CSc1nc(N)cc(Oc2ccc(-c3nnc(Nc4cccc5c4CCCC5)[nH]3)cc2)n1]

6-(4-{5-[3,4-dimethylphenyl)amino]-4H-1,2,4-triazol-3-yl}phenoxy)-2-(methylsulfanyl)pyrimidin-4-amine (also called “H3-9”), having the structure:

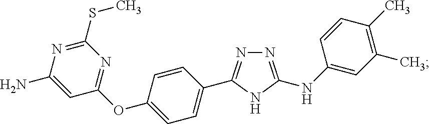 [SMILES: CSc1nc(N)cc(Oc2ccc(-c3nnc(Nc4ccc(C)c(C)c4)[nH]3)cc2)n1]

6-(4-{5-[(3,5-dimethylphenyl)amino]-4H-1,2,4-triazol-3-yl}phenoxy)-2-(methylsulfanyl)pyrimidin-4-amine (also called “H3-7”), having the structure:

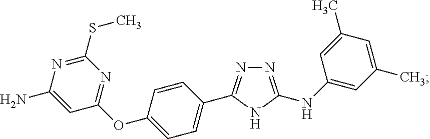 [SMILES: CSc1nc(N)cc(Oc2ccc(-c3nnc(Nc4cc(C)cc(C)c4)[nH]3)cc2)n1]

6-(4-{5-[(2,5-dimethylphenyl)amino]-4H-1,2,4-triazol-3-yl}phenoxy)-2-(methylsulfanyl)pyrimidin-4-amine (also called “H3-3”), having the structure:

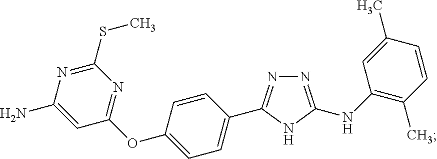 [SMILES: CSc1nc(N)cc(Oc2ccc(-c3nnc(Nc4cc(C)ccc4C)[nH]3)cc2)n1]

2-ethoxy-6-[4-(5-{[3-(trifluoromethyl)phenyl]amino}-4H-1,2,4-triazol-3-yl)phenoxy]pyrimidin-4-amine (also called “K1-72”), having the structure:

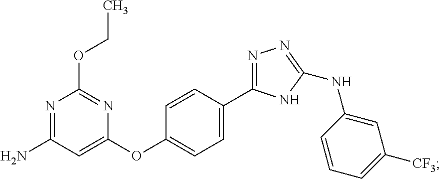 [SMILES: CCOc1nc(N)cc(Oc2ccc(-c3nnc(Nc4cccc(C(F)(F)F)c4)[nH]3)cc2)n1]

and/or

any one of a genus of compounds having a structural Formula I:

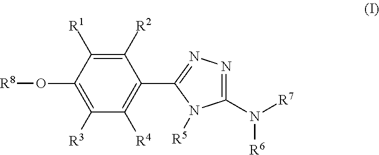

wherein:
R¹-R⁶ are independently selected from the group consisting of hydrogen, deuterium, alkyls, alkenyls, alkynyls, aryls, hetero-alkyls, hetero-alkenyls, hetero-alkynyls, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, thiols, sulfhydryls, sulfides, disulfides, sulfinyls, sulfos, thiocyanates, isothiocyanates, carbonothioyls, phosphinos, phosphonos, phosphates, silyls, and Si(OH)₃; and
R⁷-R⁸ are independently selected from the group consisting of cycloalkyl, cycloalkenyl, cycloalkynyl, aryl, heterocyle, and extended ring system.

In one embodiment, the invention provides a genus of compounds having a structural Formula I as defined above, with the proviso that the compound or specie is not a 6-(4-(5-(3-(trifluoromethyl)phenylamino)-4H-1,2,4-triazol-3-yl)phenoxy)-2-(methylthio)pyrimidin-4-amine (also can be designated “KG5”).

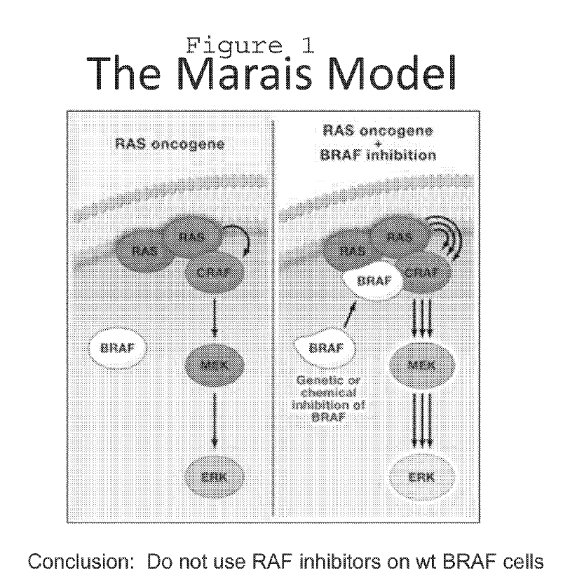

FIG. 1 illustrates a mechanism of tumorigenesis mediated by kinase-dead BRAF in the presence of oncogenic RAS; Heidorn (2010) Cell, January 22; 140(2): 209-221, demonstrated that drugs that selectively inhibit BRAF drive RAS-dependent BRAF binding to CRAF, CRAF activation, and MEK-ERK signaling. This does not occur when oncogenic BRAF is inhibited, demonstrating that BRAF inhibition per se does not drive pathway activation; it only occurs when BRAF is inhibited in the presence of oncogenic RAS. Kinase-dead BRAF mimics the effects of the BRAF-selective drugs and kinase-dead Braf and oncogenic Ras cooperate to induce melanoma in mice.

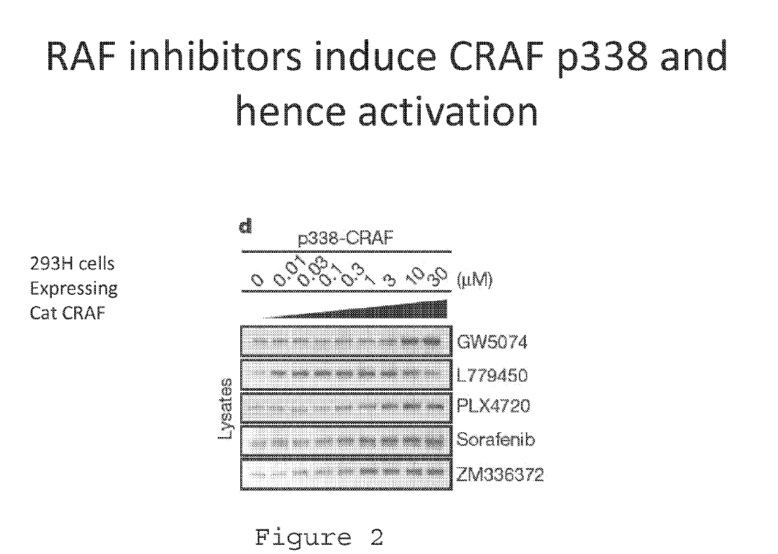

FIG. 2 illustrates that RAF inhibitor induces the active, phosphorylated state of wildtype and kinase-dead RAF, as described by Poulikakos (2010) Nature, Vol. 464, 18 Mar. 2010, doi:10.1038/nature08902; 293H cells overexpressing catC were treated with the indicated amounts of PLX4720 for 1 h. Cells were lysed, catC was immunoprecipitated (IP), washed extensively and subjected to kinase assay. Kinase activity was determined by immunoblotting for pMEK; the illustration of FIG. 2 shows the 293H cells immunoblotted for pS338-CRAF; phosphorylation at S338 steadily increased, even when concentrations were reached that inhibited MEK/ERK.

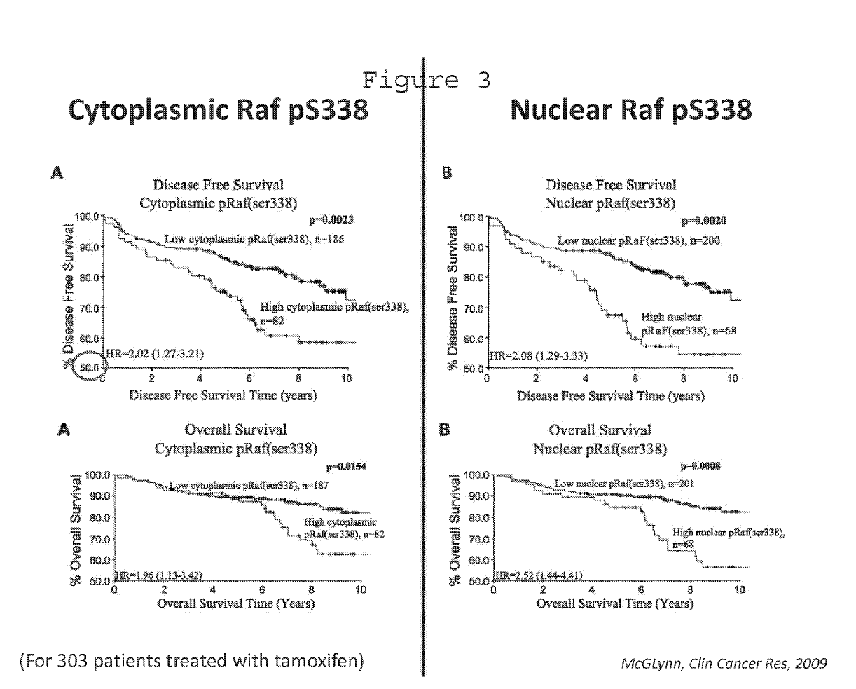

FIG. 3 illustrates data showing Tamoxifen-only treated patients disease free survival (DFS) curves, from McGlynn (2009) Clin. Cancer Res. 15:1487-1495; Published online Feb. 19, 2009. Kaplein-Meier survival curves showing DFS in patients treated only with tamoxifen whose tumors express pRaf(ser338) and pMAPK. FIG. 3A, survival curve showing a significant reduction in DFS time in patients whose tumors express high levels of cytoplasmic pRaf(ser338) (P=0.0023). FIG. 3B, survival curve showing a significant reduction in disease survival time in patients whose tumors express high levels of nuclear pRaf(ser338) (P=0.0020). High levels were defined as scores z upper quartile value. P values represent log rank testing of the differences in survival. HR, hazard ratio.

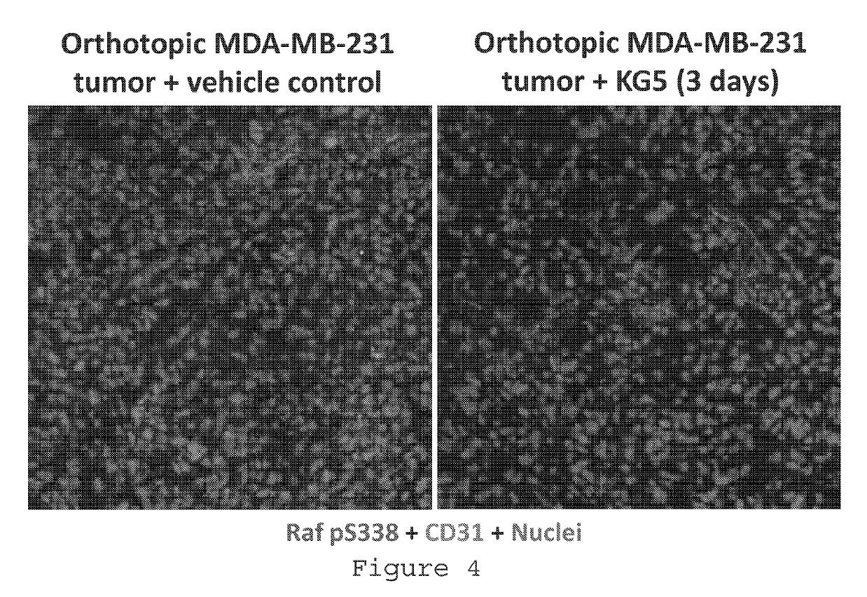

FIG. 4 illustrates an immunofluorescent staining of phospho-S338 c-RAF (CRAF) in orthotopic breast cancer tissues untreated or systemically treated with KG5 for 3 days. Conclusion: KG5 treatment inhibits serine 338 phosphorylation of CRAF in orthotopic tumors implanted in mice.

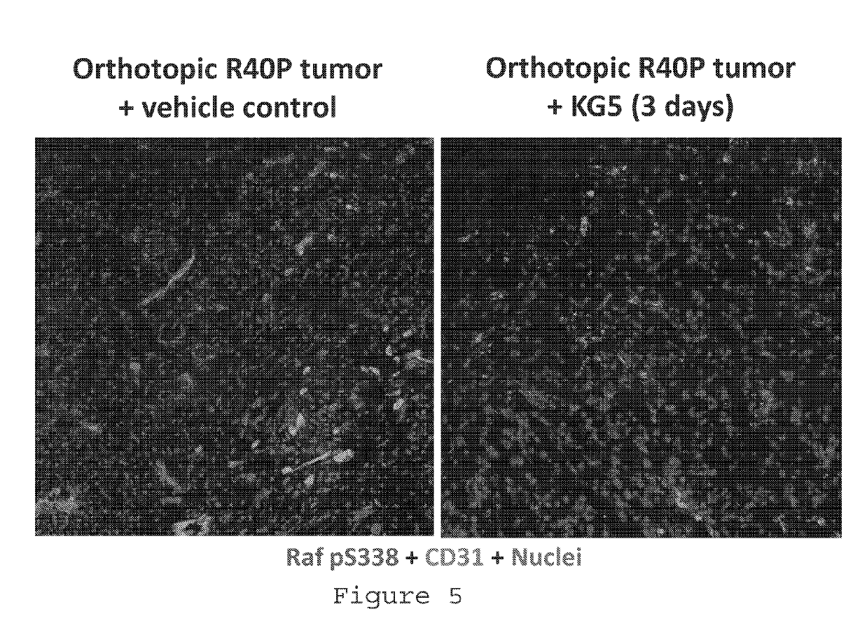

FIG. 5 illustrates an immunofluorescent staining of phospho-S338 CRAF in orthotopic pancreatic cancer tissues untreated or systemically treated with KG5 for 3 days. Conclusion: KG5 treatment inhibits serine 338 phosphorylation of CRAF in orthotopic tumors implanted in mice.

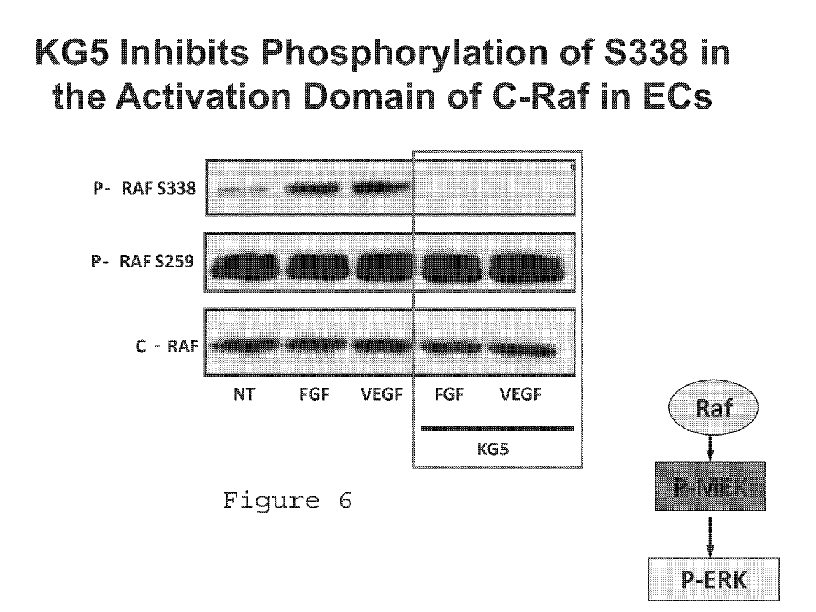

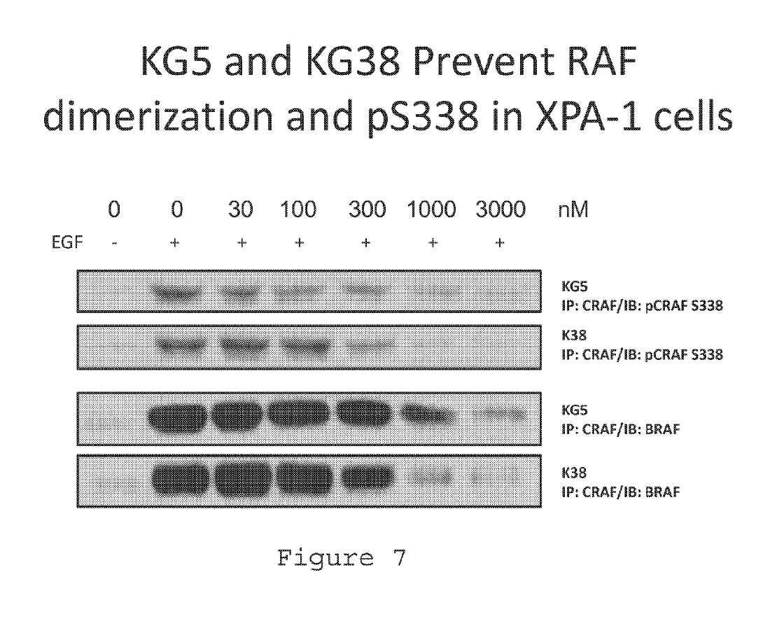

FIGS. 6 and 7 illustrate immunoblot analysis of CRAF immunoprecipitated from XPA-1 cells treated with KG5 and KG38. Conclusion: KG5 and KG38 prevent RAF dimerization and serine 338 phosphorylation on CRAF.

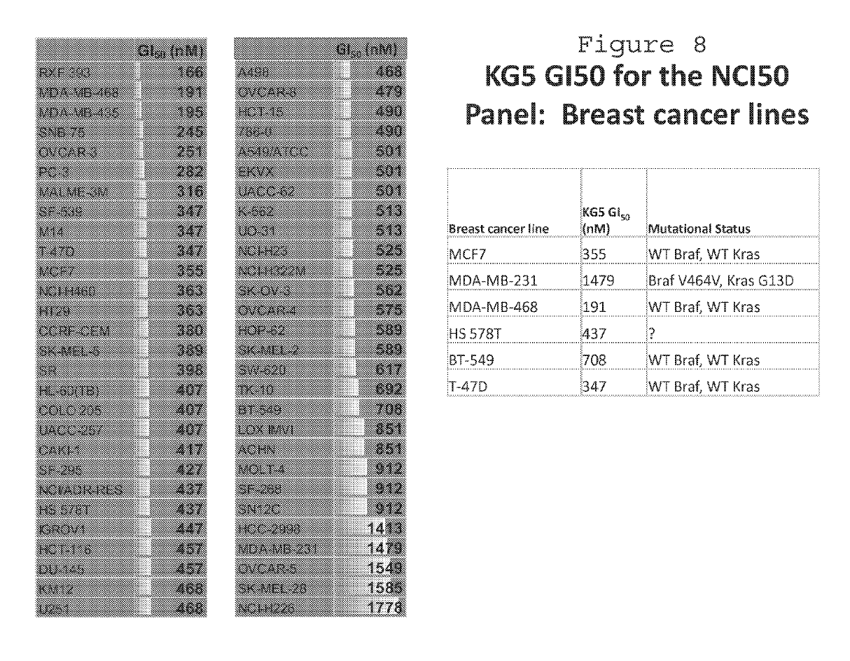

FIG. 8 illustrates NCI-50 panel showing the half maximal growth inhibitory concentration of KG5 in 50 human cancer cell lines. Conclusion: KG5 inhibits the growth of several human cancer cell lines.

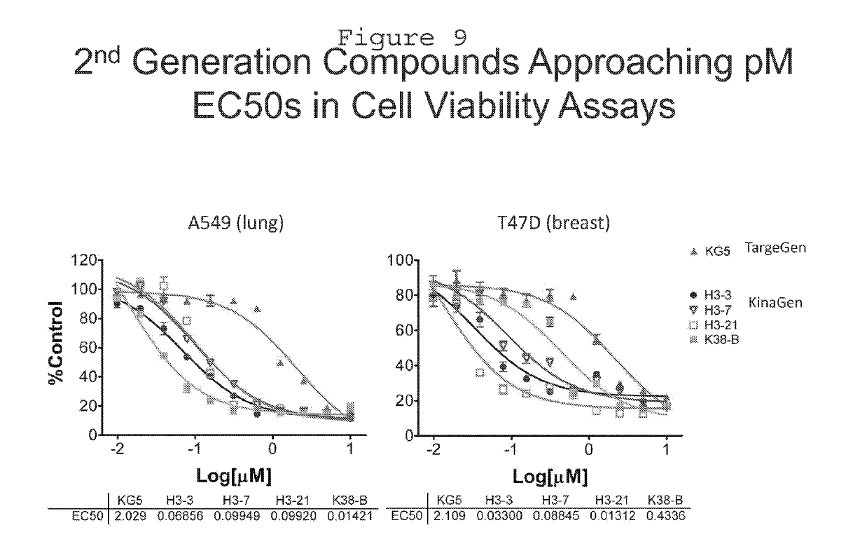

FIG. 9 illustrates cell viability assays and EC50s performed in human lung (A549) and breast (T47D) carcinoma cell lines treated with KG5 and the second generation compounds: H3-3, H3-7, H3-21, K38-B. Conclusion: All compounds decrease cell viability. The second generation compounds are more potent than KG5.

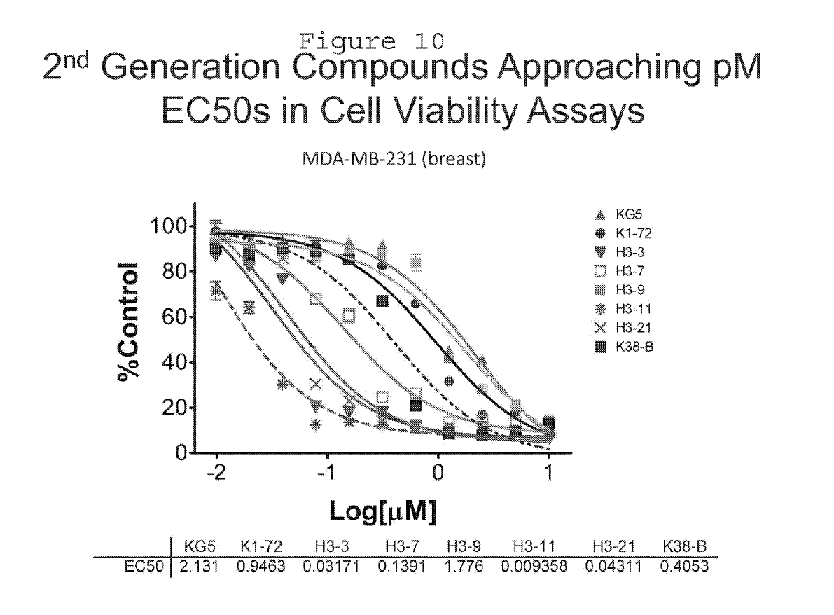

FIG. 10 illustrates cell viability assays and EC50s performed in human breast (MDA-MB-231) carcinoma cell line treated with KG5 and the second generation compounds: K1-72, H3-3, H3-7, H3-9, H3-11, H3-21, K38-B. Conclusion: All compounds decrease cell viability. The second generation compounds are more potent than KG5.

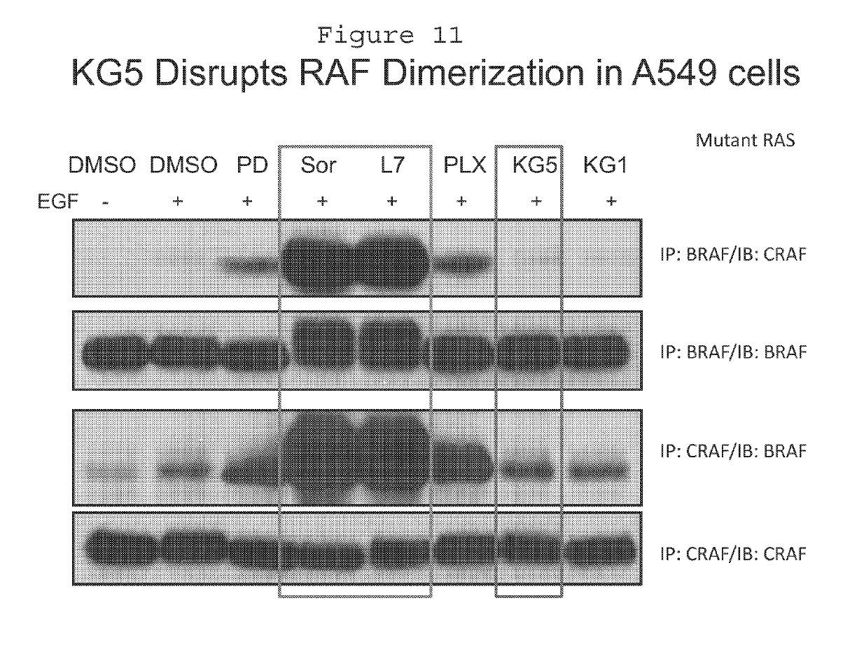

FIG. 11 illustrates immunoblot analysis of BRAF and CRAF immunoprecipitates from A549 cells treated with PD0325901, Sorafenib, L-779450, PLX4720, KG5 or KG1. Conclusion: KG5 prevents RAF dimerization.

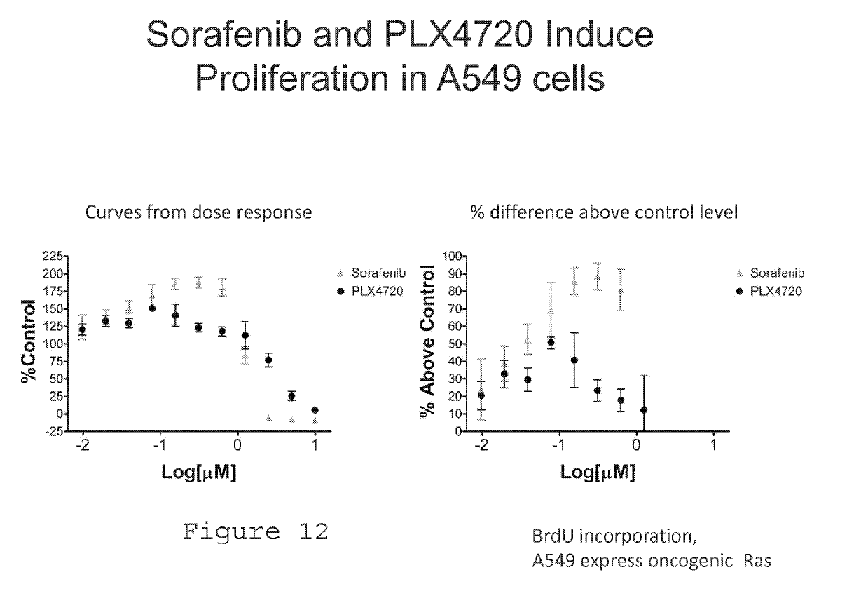

FIG. 12 illustrates cell proliferation assays performed in A549 cells treated with Sorafenib or PLX4720. Conclusion: Sorafenib and PLX4720 induce proliferation in A549 cells.

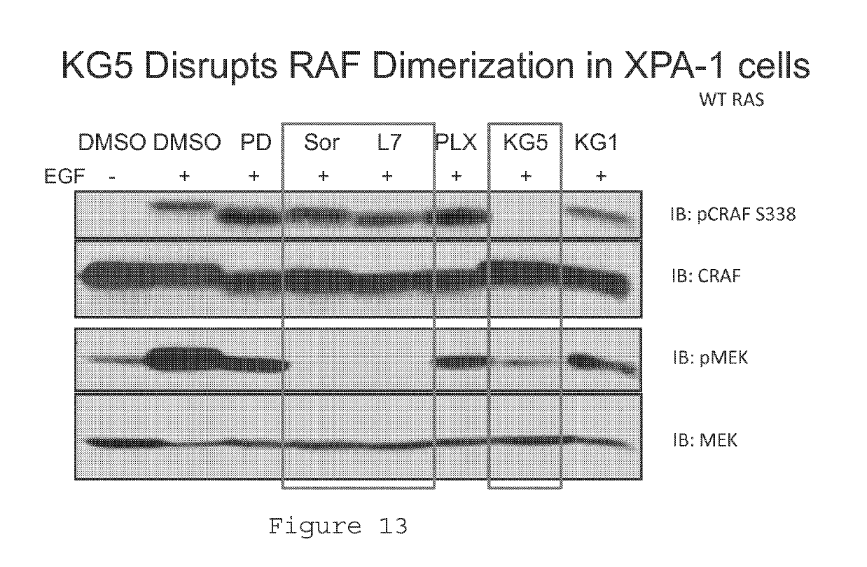

FIG. 13 illustrates immunoblot analysis of phospho-5338 CRAF, phospho-MEK, total CRAF and total MEK of lysates from XPA-1 cells treated with PD0325901, Sorafenib, L-779450, PLX4720, KG5 or KG1. Conclusion: KG5 inhibits phosphorylation of CRAF on serine 338.

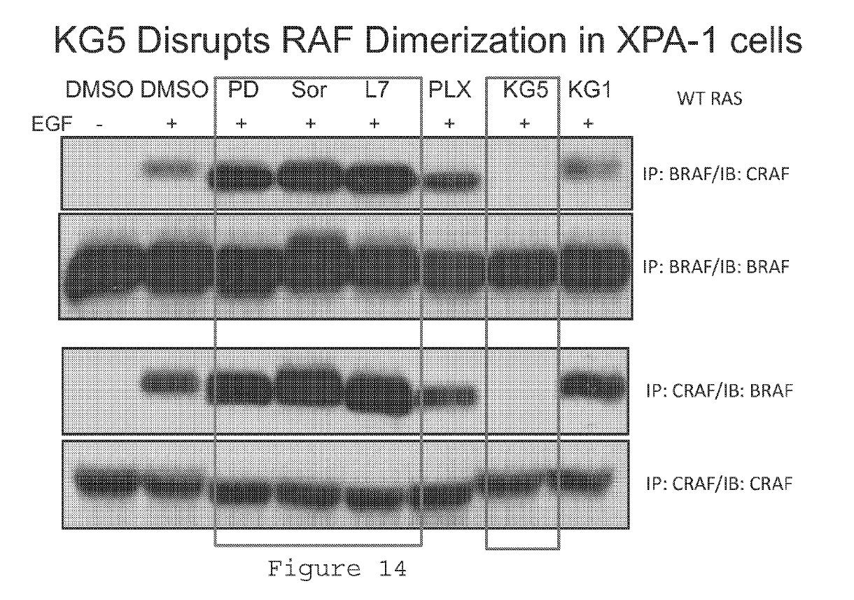

FIG. 14 illustrates immunoblot analysis of BRAF and CRAF immunoprecipitates from XPA-1 cells treated with PD0325901, Sorafenib, L-779450, PLX4720, KG5 or KG1. Conclusion: KG5 prevents RAF dimerization.

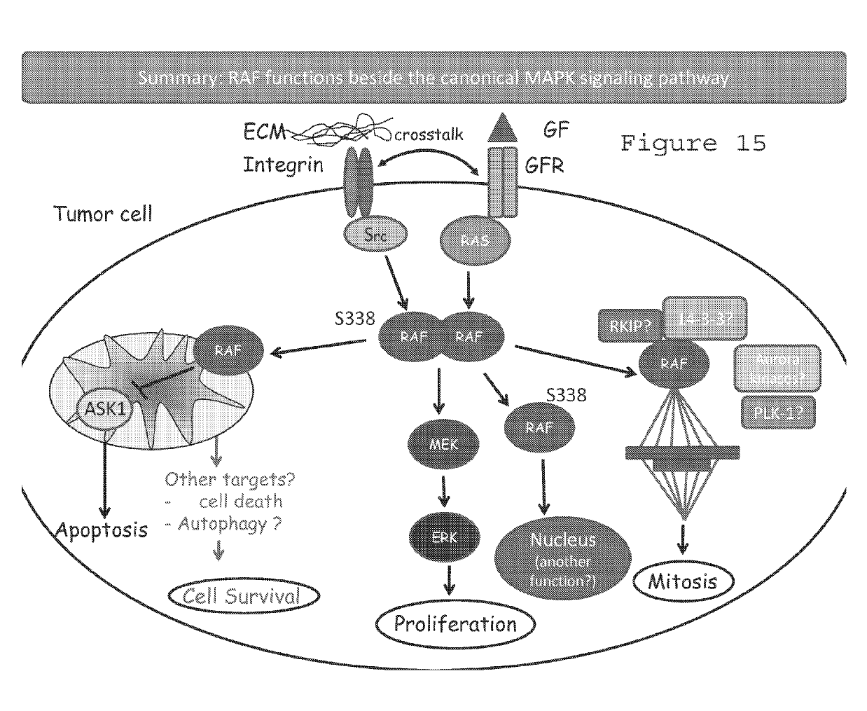

FIG. 15 schematically illustrates RAF functions other than the canonical MAPK signaling pathway; it is a schematic depicting the MEK-independent functions of c-RAF in mitosis and survival.

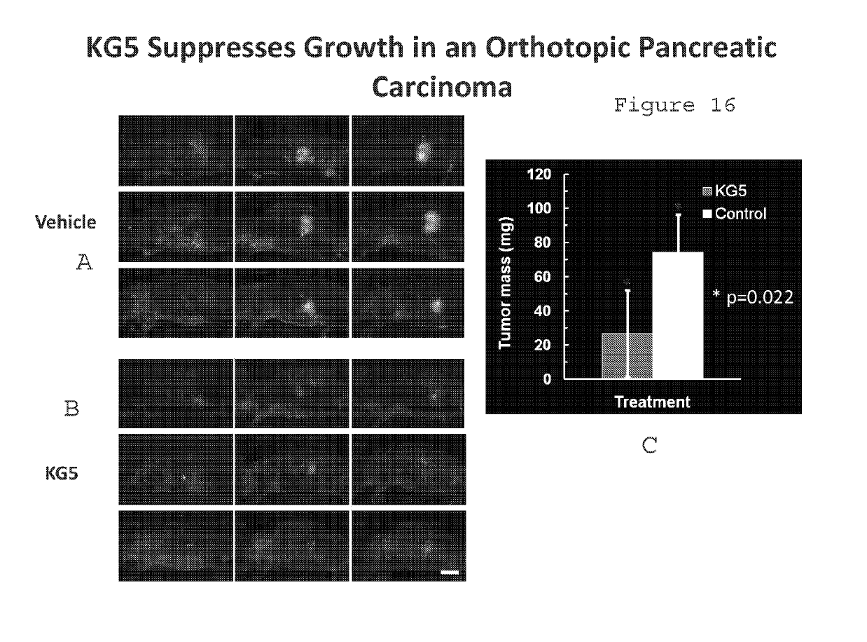

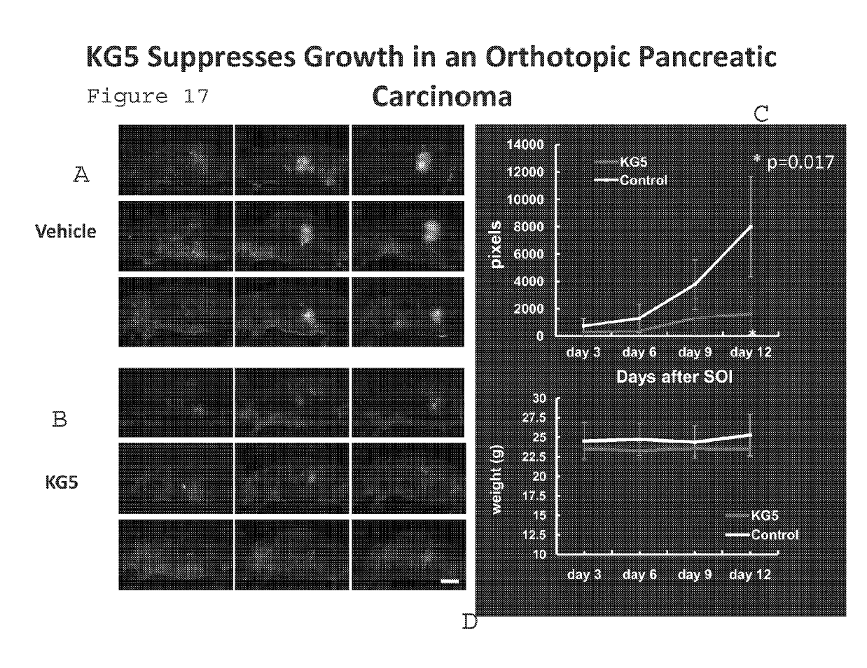

FIG. 16 and FIG. 17 illustrate data showing that KG5 inhibits pancreatic tumor growth and reduces tumor vascular density, as described in Murphy (2010) Proc. Natl. Acad. Sci. USA, Mar. 2, 2010; vol. 107(9): 4299-4304. FIGS. 16A and 16B, and FIGS. 17A and 17B: Real-time fluorescent imaging of XPA-1-RFP pancreatic tumor xenografts in the pancreas of Nestin-GFP mice (n=5/group) treated with either vehicle (FIG. 16A, 17A) or KG5 (FIG. 16B, 17B) (50 mg/kg, i.p., bid). Drug treatments were started 3 days after surgical orthotopic implantation (SOT) of XPA-1-RFP tumors, and tumor progression was monitored every 3 days by whole-animal imaging (Scale bar, 10 mm). FIG. 17(C) graphically illustrates a plot of tumor surface area over time for the vehicle- and KG5-treated groups, Tumor surface area was calculated by adding the total pixels of both the ventral and lateral images of the tumor. *P=0.034. FIG. 17(D) graphically illustrates average body weight of the mice measured each day of whole-animal imaging described in A and B. FIG. 16(C) graphically illustrates total weights of resected primary tumors on day 15 post-SOT. *P=0.022.

Animals were systemically treated with either vehicle or KG5 (50 mg/kg, bid) beginning 3 days after surgical orthotopic implantation (SOI) of a tumor fragment. Tumor growth was monitored noninvasively by whole-body imaging. Tumor growth was completely suppressed in animals treated with KG5 compared to vehicle alone 12 days post-SOI. Representative time-course images (lateral view) from three animals demonstrate that the growth of pancreatic tumors treated with 6 was suppressed and RFP intensity was abolished by day 12 after SOI compared to vehicle-treated animals (FIGS. 16A and 16B, and FIGS. 17A and 17B). Clearly, KG5 suppressed tumor growth in this model (FIG. 17C) but caused no weight loss, and the mice did not demonstrate any signs of lethargy. Animals treated with KG5 produced an average tumor weight of 26.7 mg compared to 74.1 mg for vehicle-treated animals (FIG. 16C). At this time, GFP-labeled blood vessels were imaged and tumor-associated blood vessel density was quantified by measuring the ratio of total blood vessel length to tumor volume. Tumors treated with 6 were substantially less vascularized relative to vehicle treatment, and images of the GFP-labeled tumor vasculature showed a significant reduction in the total blood vessels present. The mean vessel length/tumor volume was 2.5 mm/mm³ compared to 0.2 mm/mm³ for vehicle and KG5, respectively.

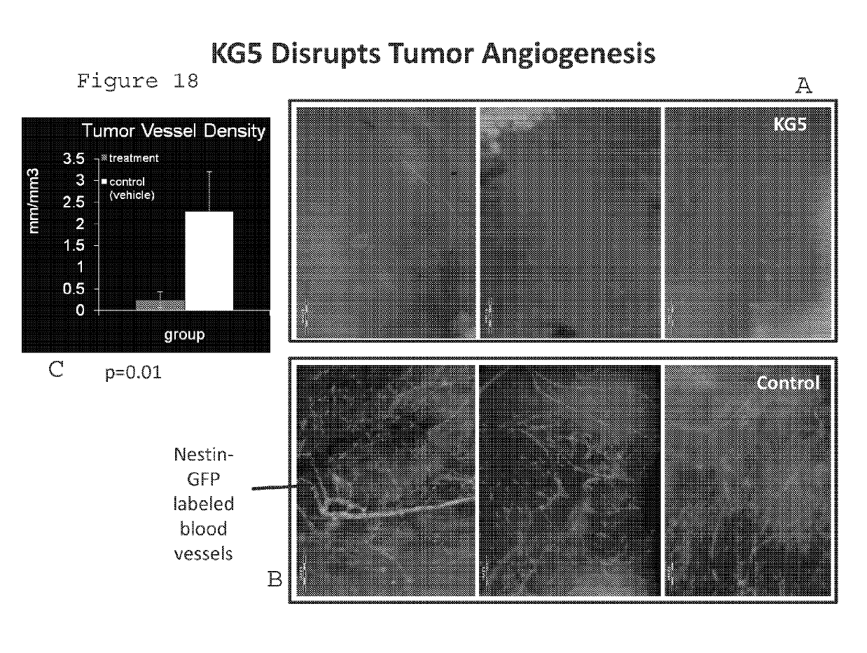

FIG. 18 further illustrates data from the studies of FIG. 16 and FIG. 17: FIG. 18(A) and FIG. 18(B) illustrate representative fluorescent images of endothelial GFP expression (GFP expression driven by the Nestin promoter) within the XPA-1-RFP tumors after resection. Images were taken 15 days post-SOI (Scale bar, 200 μm); FIG. 18(C) illustrates a plot of tumor vessel density from images acquired as in E, Blood vessels imaged as in E were converted to length (mm) and normalized to tumor volume (mm³). *P=0.01.

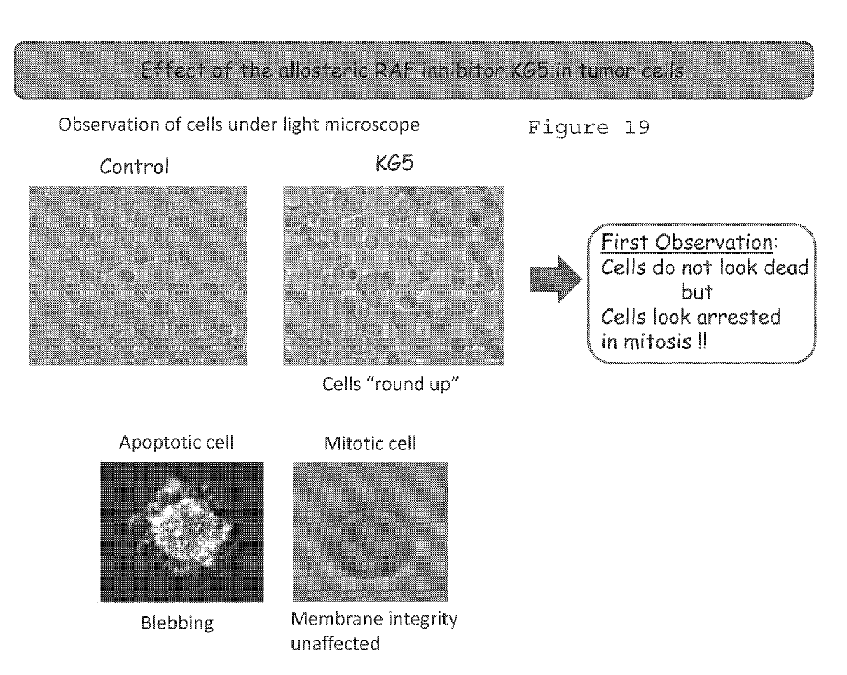

FIG. 19 illustrates bright field images of XPA-1 cells treated for 20 hours with the allosteric RAF inhibitor KG5. Conclusion: Upon initial exposure to KG5 cells look arrested in mitosis.

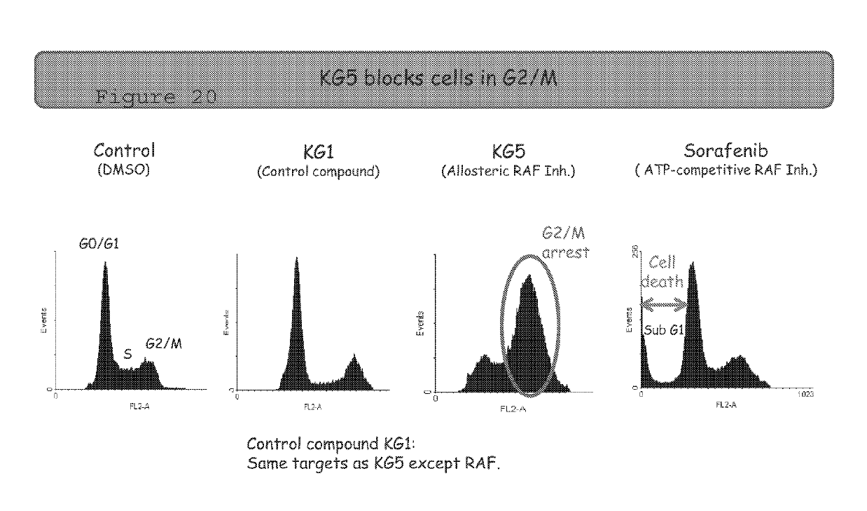

FIG. 20 illustrates cell cycle analysis of XPA-1 cells treated for 20 hours with the allosteric RAF inhibitor KG5. Conclusion: Upon initial exposure to KG5 cells become arrested in G2/M.

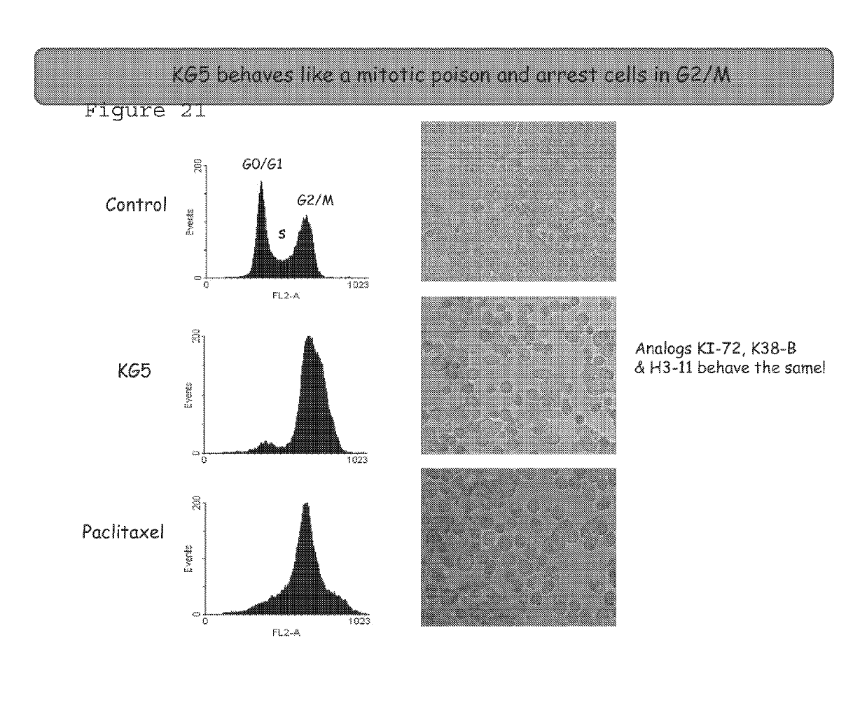

FIG. 21 illustrates cell cycle analysis of XPA-1 cells treated for 20 hours with the allosteric RAF inhibitor KG5 or the microtubule stabilizer paclitaxel. Conclusion: KG5 behaves like a mitotic poison and arrests cells in G2/M.

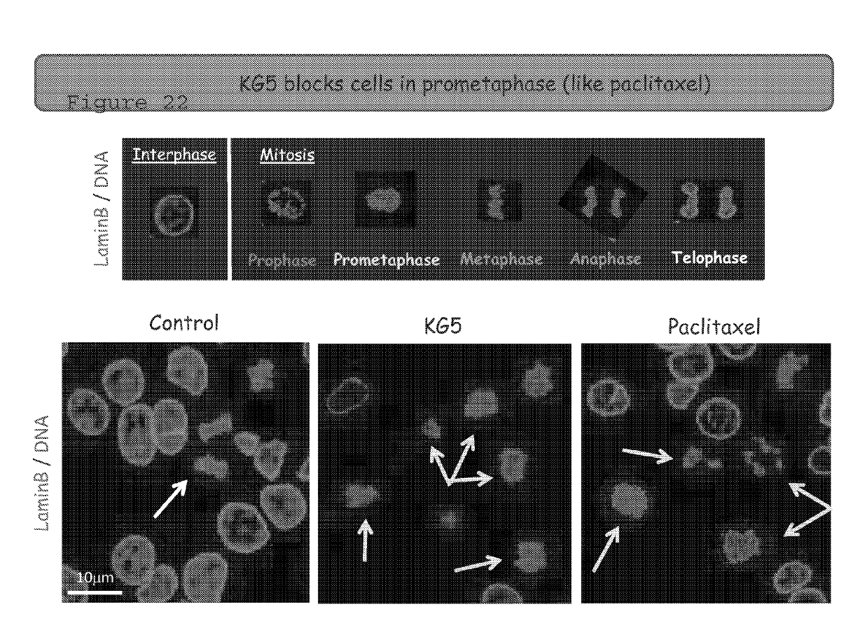

FIG. 22 illustrates confocal microscopy images of XPA-1 cells treated for 20 hours with KG5 or paclitaxel. Conclusion: KG5, like paclitaxel arrests cells at pro-metaphase.

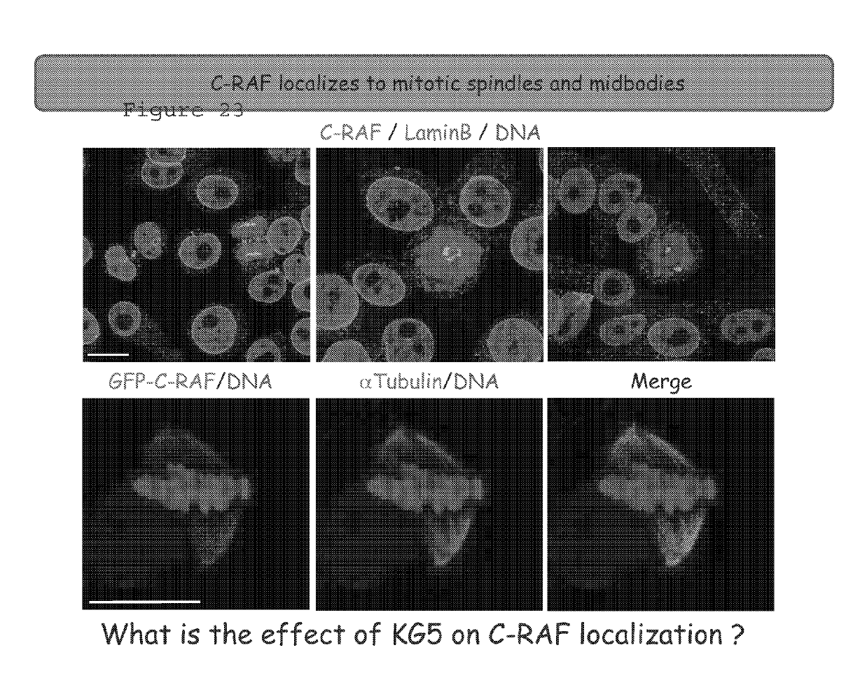

FIG. 23 illustrates confocal microscopy images of XPA-1 cells. Conclusion: CRAF localizes to the mitotic spindle and midbodies.

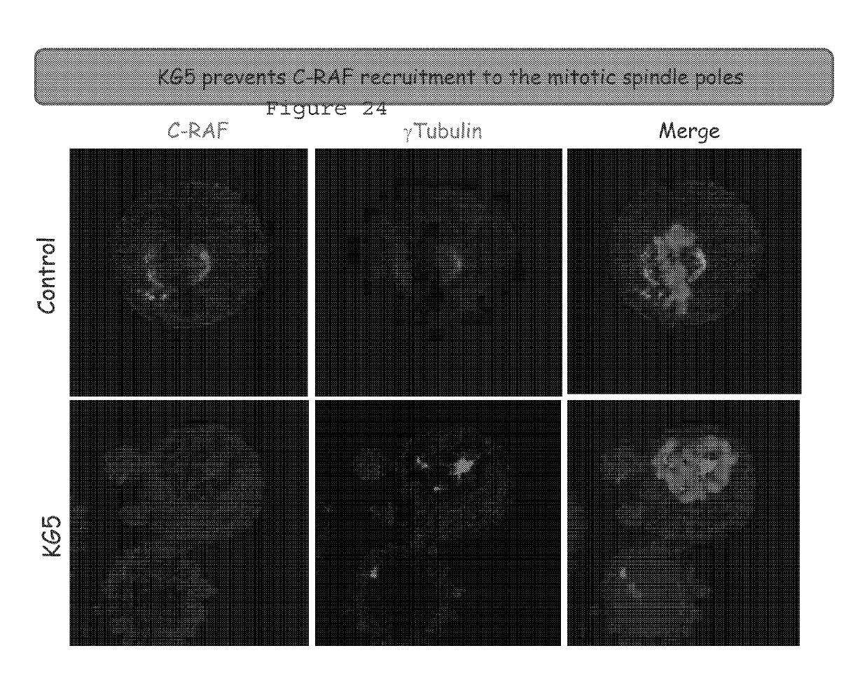

FIG. 24 illustrates confocal microscopy images of XPA-1 cells treated with KG5. Conclusion: KG5 prevents CRAF recruitment to the mitotic spindle.

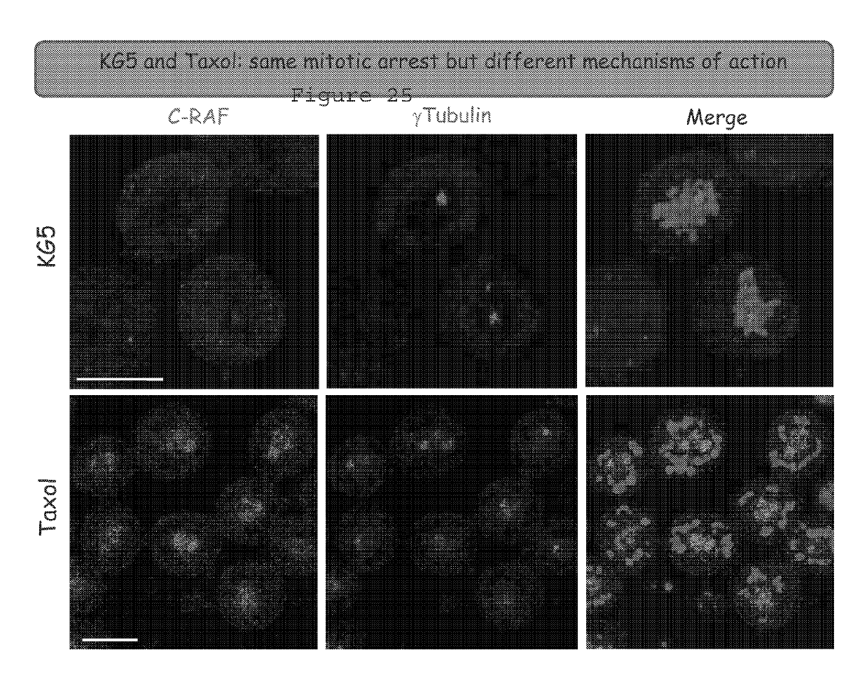

FIG. 25 illustrates confocal microscopy images of XPA-1 cells treated with KG5 or paclitaxel. Conclusion: KG5 and paclitaxel arrest cells at prometaphase but they do so by different mechanisms since paclitaxel does not affect CRAF localization to the mitotic spindle whereas KG5 does.

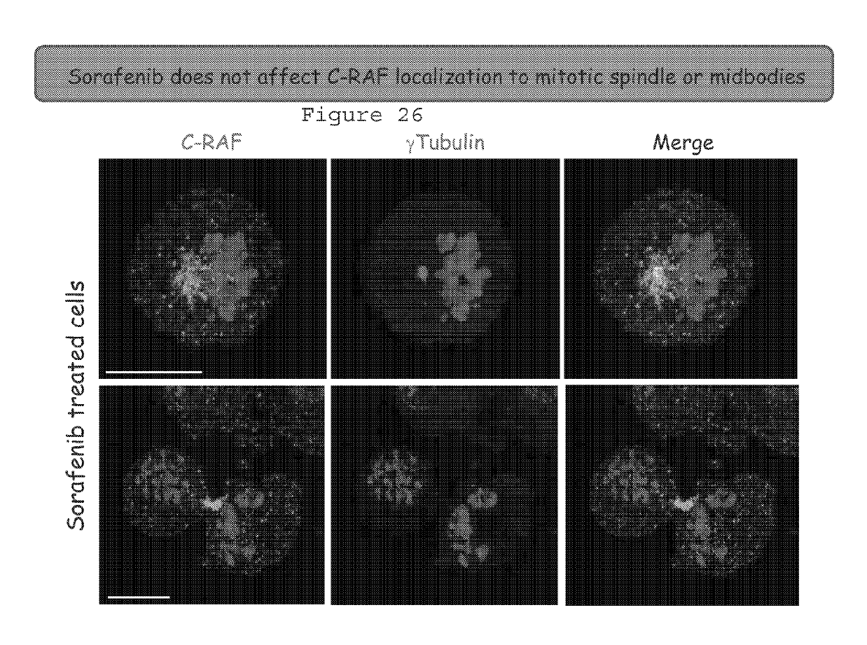

FIG. 26 illustrates confocal microscopy images of XPA-1 cells treated with sorafenib. Conclusion: The ATP-competitive RAF inhibitor Sorafenib does not arrest cells at pro-metaphase and does not prevent CRAF localization to the mitotic spindle or midbodies.

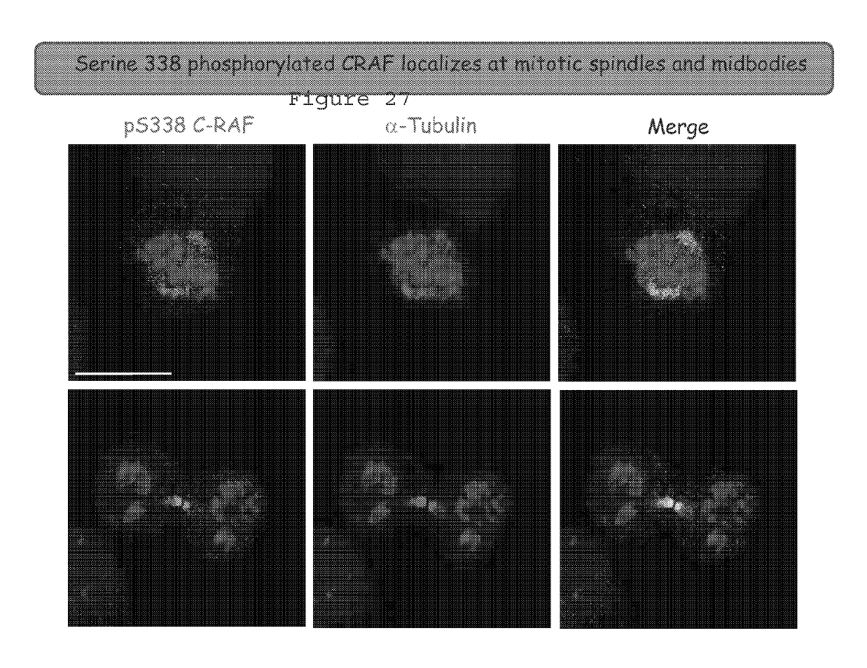

FIG. 27 illustrates confocal microscopy images of XPA-1 cells. Conclusion: phospho-S338 CRAF localizes to mitotic spindles and midbodies.

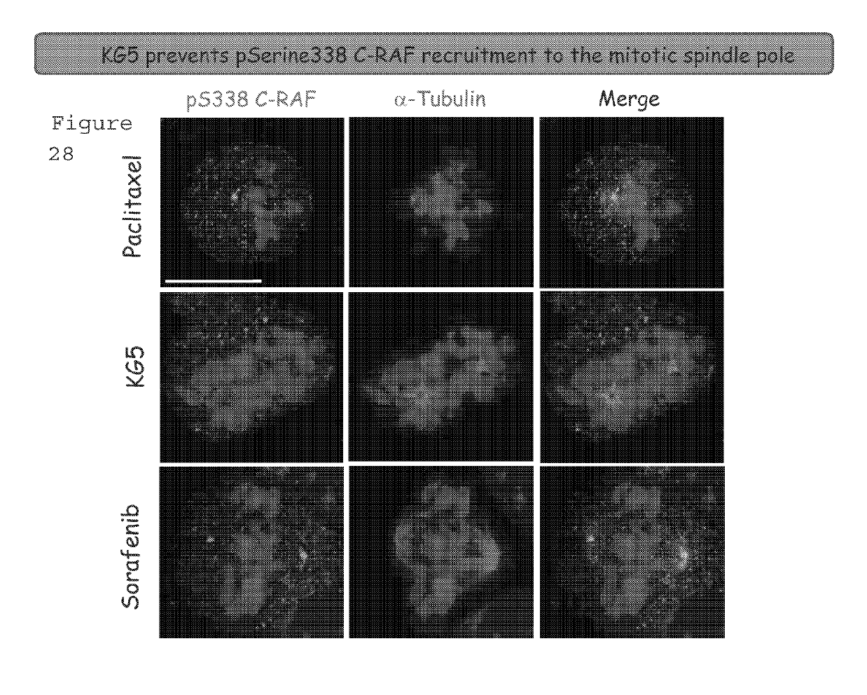

FIG. 28 illustrates confocal microscopy images of XPA-1 cells treated with sorafenib, KG5 or paclitaxel. Conclusion: KG5 but not Sorafenib nor paclitaxel, prevents phospho-S338 CRAF recruitment to the mitotic spindle.

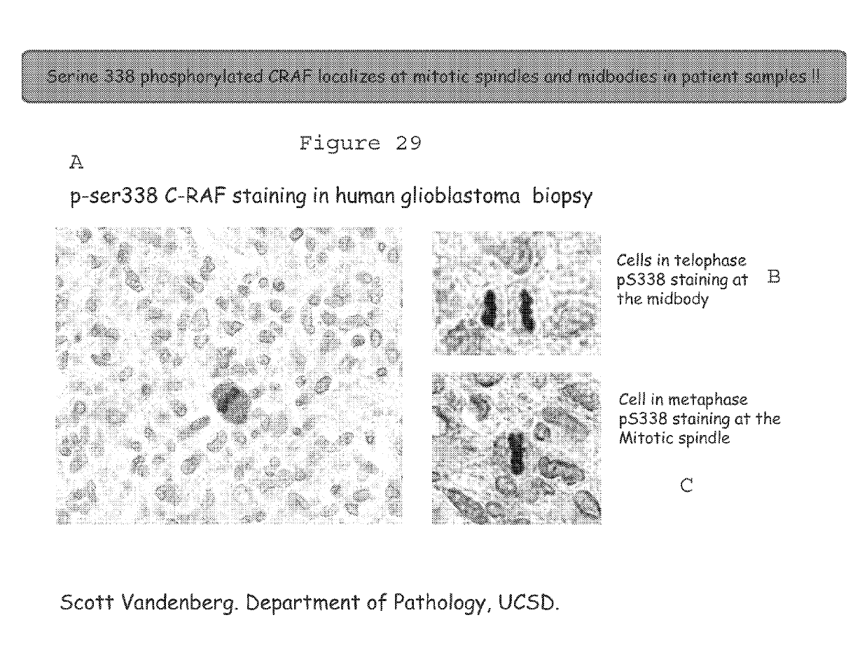

FIG. 29 illustrates immunohistochemical analysis of phospho-S338 CRAF performed in biopsies from glioblastoma patients. Conclusion: Phospho-S338 CRAF localizes to the mitotic spindle of tumor dividing cells in biopsies from cancer patients.

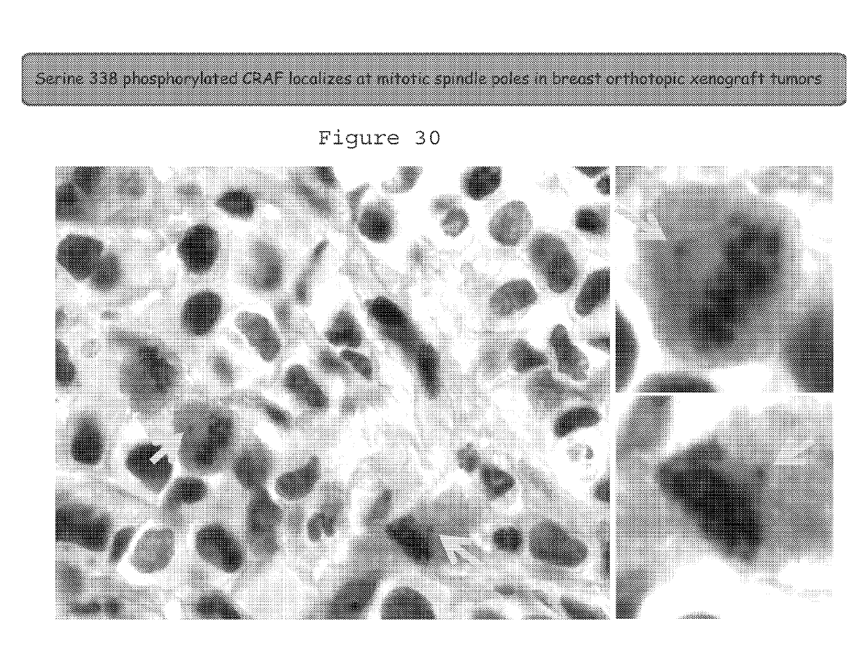

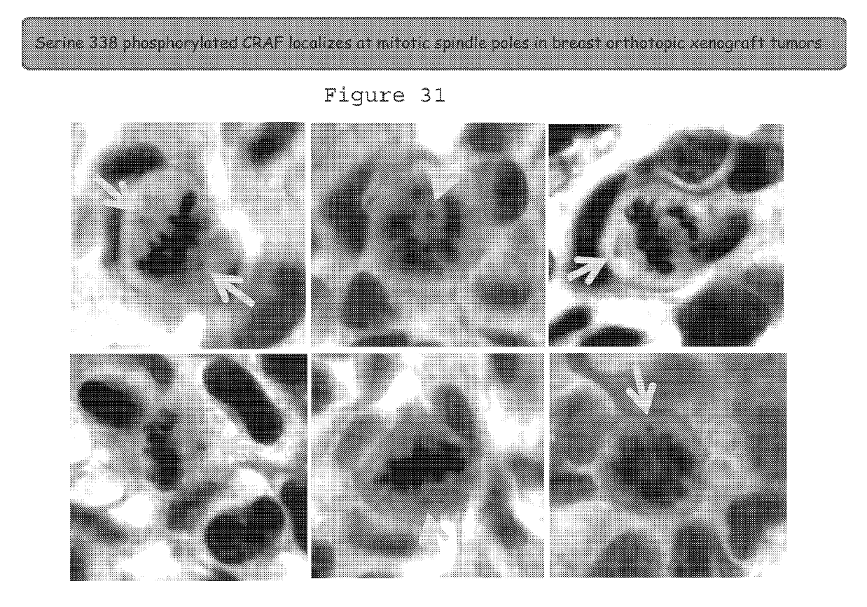

FIG. 30 and FIG. 31 illustrate immunohistochemical analysis of phospho-S338 CRAF performed in tissues from orthotopic breast tumors implanted in mice. Conclusion: Phospho-S338 CRAF localizes to the mitotic spindle of tumor dividing cells in tissues derived from breast orthotopic xenograft tumors.

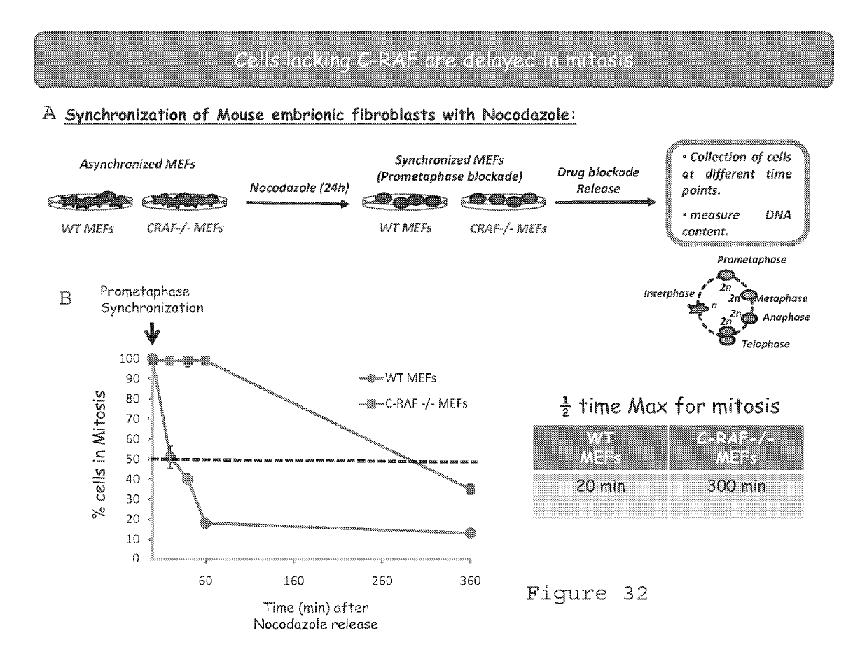

FIG. 32 illustrates WT and Craf^{-/-} MEFs were synchronized at pro-metaphase with a thymidine-nocodazole block and subsequently released from the blockade and allowed to progress through mitosis. Quantification of cells in G2/M was performed by flow cytometry. Conclusion: Cells lacking CRAF are delayed in mitosis.

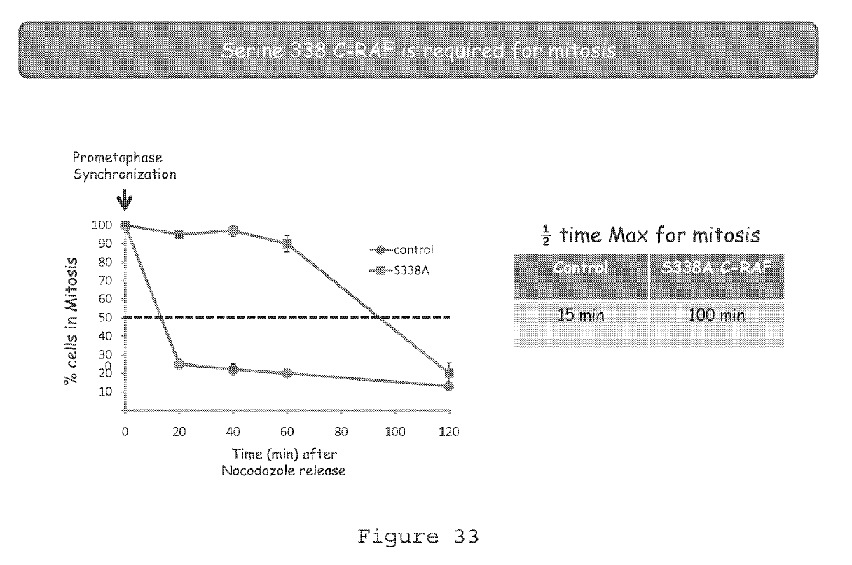

FIG. 33 illustrates XPA-1 cells ectopically expressing either WT RAF or S338A mutant CRAF were arrested in pro-metaphase and subsequently allowed to progress through mitosis. Quantification of cells in G2/M was performed by flow cytometry. Conclusion: phosphorylation of CRAF on serine 338 is required for mitosis.

### Example 2

### Methods of the Invention are Effective for Inhibiting and/or Promoting Cell Growth and Arresting Mitosis

The data presented herein demonstrates methods of the invention are effective for arresting mitosis and/or inhibiting or decreasing cell growth.

This study used inter alia as an allosteric inhibitor of c-RAF (compound 6, also referred to here as KG5, as described by Murphy (2010) “Disruption of angiogenesis and tumor growth with an orally active drug that stabilizes the inactive state of PDGFRbeta/B-RAF”, Proc. Natl. Acad. Sci. USA 107:4299-4304) that does not compete for ATP but inhibits phospho-5338 CRAF and tumor growth in mice.

KG5 exerted a broad growth inhibitory activity against the NCI-60 panel (Table 1, below) suggesting that KG5 influences a general mechanism of tumor cell growth or survival that is independent of the activation status of RAS, RAF, or other effectors. Table 1 summarizes KG5's effect in 60 NCI cancer cell lines: the NCI-60 panel showing the half maximal growth inhibitory concentration of KG5 and the RAS and BRAF status in 60 human cancer cell lines.

TABLE 1

| | | Mutated | Mutated | |
|---|---|---|---|---|
| Cell line | | RAS | BRAF | GI50 (nM) |
| Breast Cancer | BT-549 | No | No | 708 |
| Breast Cancer | HS 578T | Yes | No | 437 |
| Breast Cancer | MCF7 | No | No | 355 |
| Breast Cancer | MDA-MB-231/ATCC | Yes | Yes | 1479 |
| Breast Cancer | MDA-MB-468 | No | No | 191 |
| Breast Cancer | T-47D | Yes | No | 347 |
| CNS Cancer | SF-268 | No | No | 912 |
| CNS Cancer | SF-295 | No | No | 427 |
| CNS Cancer | SF-539 | No | No | 347 |
| CNS Cancer | SNB-75 | No | No | 245 |
| CNS Cancer | U251 | No | No | 468 |
| Colon Cancer | COLO 205 | Yes | No | 407 |
| Colon Cancer | HCC-2998 | No | No | 1413 |
| Colon Cancer | HCT-116 | Yes | Yes | 457 |
| Colon Cancer | HCT-15 | Yes | Yes | 490 |
| Colon Cancer | HT29 | No | Yes | 363 |
| Colon Cancer | KM12 | No | No | 468 |
| Colon Cancer | SW-620 | No | No | 617 |
| Leukemia | CCRF-CEM | Yes | No | 380 |
| Leukemia | HL-60(TB) | Yes | No | 407 |
| Leukemia | K-562 | No | No | 513 |
| Leukemia | MOLT-4 | Yes | No | 912 |
| Leukemia | SR | No | No | 398 |
| Melanoma | LOX IMVI | No | Yes | 851 |
| Melanoma | M14 | No | Yes | 347 |
| Melanoma | MALME-3M | No | Yes | 316 |
| Melanoma | MDA-MB-435 | No | Yes | 195 |
| Melanoma | SK-MEL-2 | Yes | No | 589 |
| Melanoma | SK-MEL-28 | No | Yes | 1585 |
| Melanoma | SK-MEL-5 | No | Yes | 389 |
| Melanoma | UACC-257 | No | Yes | 407 |
| Melanoma | UACC-62 | No | Yes | 501 |
| NSC Lung Cancer | A549/ATCC | Yes | No | 501 |
| NSC Lung Cancer | EKVX | No | No | 501 |
| NSC Lung Cancer | HOP-62 | Yes | No | 589 |
| NSC Lung Cancer | NCI-H226 | No | No | 1778 |
| NSC Lung Cancer | NCI-H23 | Yes | No | 525 |
| NSC Lung Cancer | NCI-H322M | No | No | 525 |
| NSC Lung Cancer | NCI-H460 | Yes | No | 363 |
| Ovarian Cancer | IGROV1 | No | No | 447 |
| Ovarian Cancer | NCI/ADR-RES | No | No | 437 |
| Ovarian Cancer | OVCAR-3 | No | No | 251 |
| Ovarian Cancer | OVCAR-4 | No | No | 575 |
| Ovarian Cancer | OVCAR-5 | Yes | No | 1549 |
| Ovarian Cancer | OVCAR-8 | No | No | 479 |
| Ovarian Cancer | SK-OV-3 | No | No | 562 |
| Prostate Cancer | DU-145 | No | No | 457 |
| Prostate Cancer | PC-3 | No | No | 282 |
| Renal Cancer | 786-0 | No | No | 490 |
| Renal Cancer | A498 | No | No | 468 |
| Renal Cancer | ACHN | No | No | 851 |
| Renal Cancer | CAKI-1 | No | No | 417 |
| Renal Cancer | RXF 393 | No | No | 166 |
| Renal Cancer | SN12C | No | No | 912 |
| Renal Cancer | TK-10 | No | No | 692 |
| Renal Cancer | UO-31 | No | No | 513 |

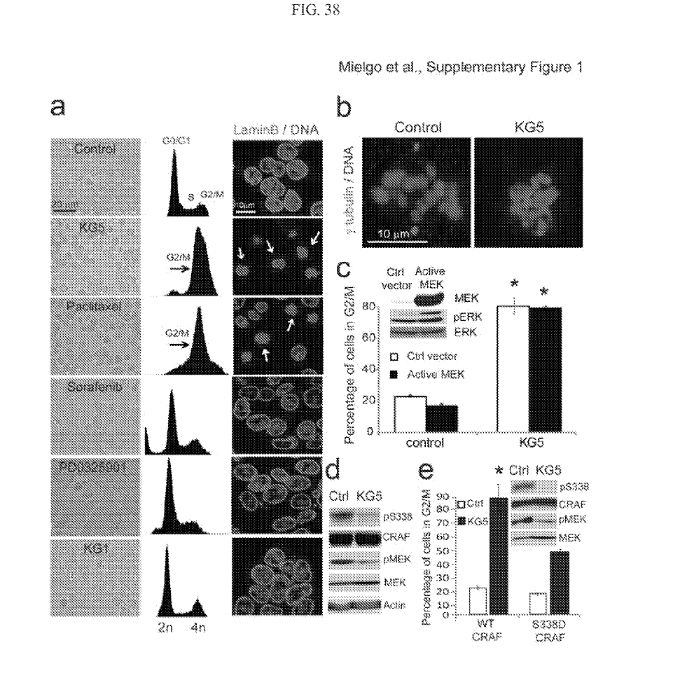

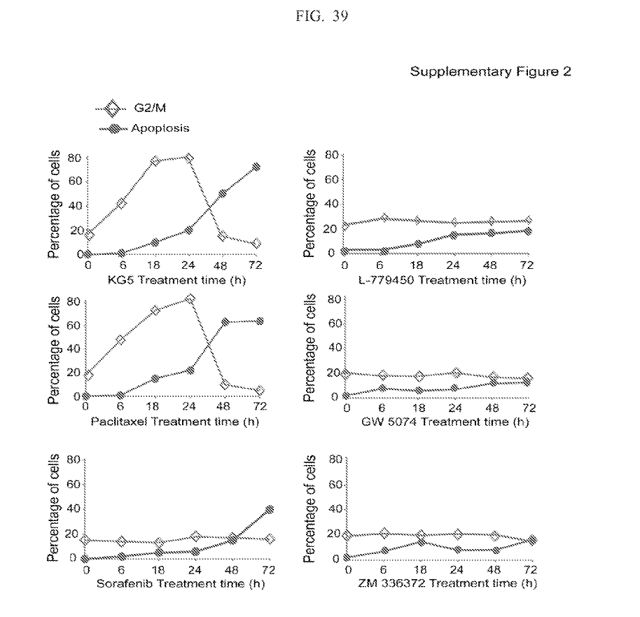

Tumor cells exposed to KG5 appeared rounded and arrested in mitosis, whereas cells treated with the same concentration of ATP-competitive RAF or MEK inhibitors, maintained their adhesive properties and normal mitotic function (FIGS. 38a and 39). KG5 treatment led to inhibition of phospho-S338 CRAF and mitotic arrest at pro-metaphase followed by cell death in all proliferating tumor cell lines tested (FIGS. 38a, 38b, 38d, 38e, and FIG. 39 and FIG. 40), mimicking the effects of paclitaxel.

As with most kinase targeted drugs, KG5 inhibits other targets besides RAF that might account for some of the anti-proliferative effects observed. To address this, we incorporated the control compound KG1, a structural analogue of KG5 that like KG5, inhibits c-Kit, Flt-3 and PDGFR but does not target RAF¹². In this case KG1 did not inhibit cell proliferation, suggesting the effects of KG5 are due to its effect on RAF (FIG. 38a).

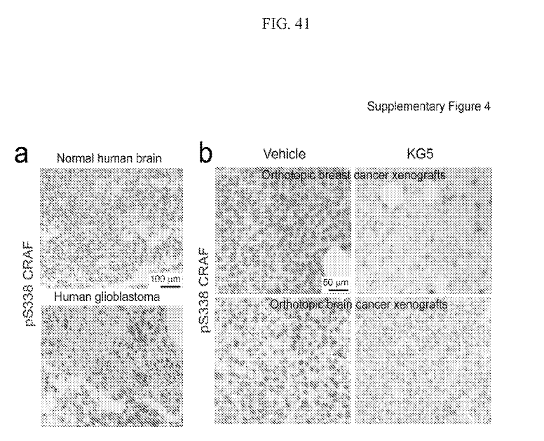

Phosphorylation of CRAF on serine 338 has been linked to cell proliferation survival as well as breast cancer progression^{9-11,14-16}. To determine whether KG5 could similarly regulate phospho-5338 RAF in tumors, we analyzed orthotopic breast and brain tumor tissues for the presence of this marker before or after systemic delivery of the drug. While chronic dosing with KG5 led to a suppression of tumor growth¹², a single systemic KG5 treatment led to a qualitative decrease in phospho-5338 RAF and an accumulation of cells at pro-metaphase in these tissues after three days (FIG. 38e and FIG. 41b). Importantly, while ectopic expression of active MEK failed to rescue the effects of KG5 on cell proliferation, cells expressing a phospho-mimetic S338D CRAF mutant showed resistance to KG5 suggesting that the anti-mitotic effects of KG5 are MEK-independent but linked to inhibition of phospho-S338 CRAF (FIGS. 38c, 38e). These findings demonstrate that c-RAF plays a MEK-independent role in cell proliferation that can be inhibited by an allosteric c-RAF inhibitor such as KG5.

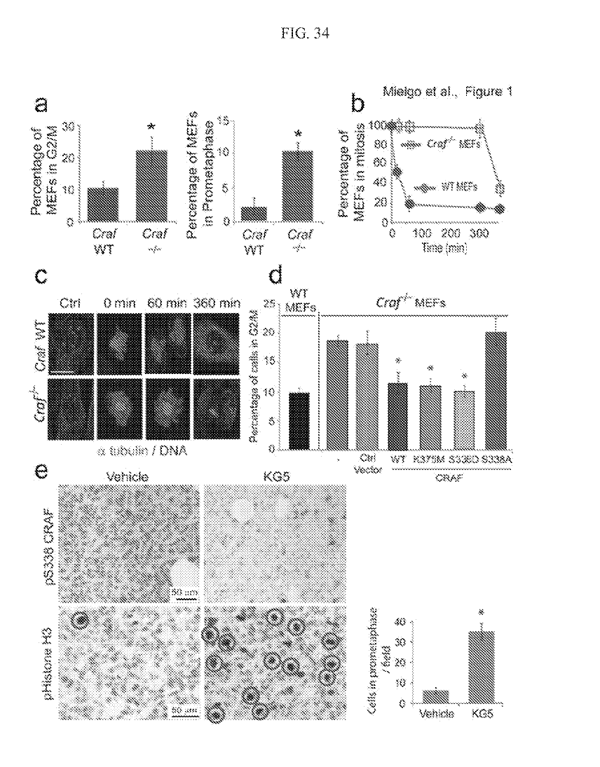

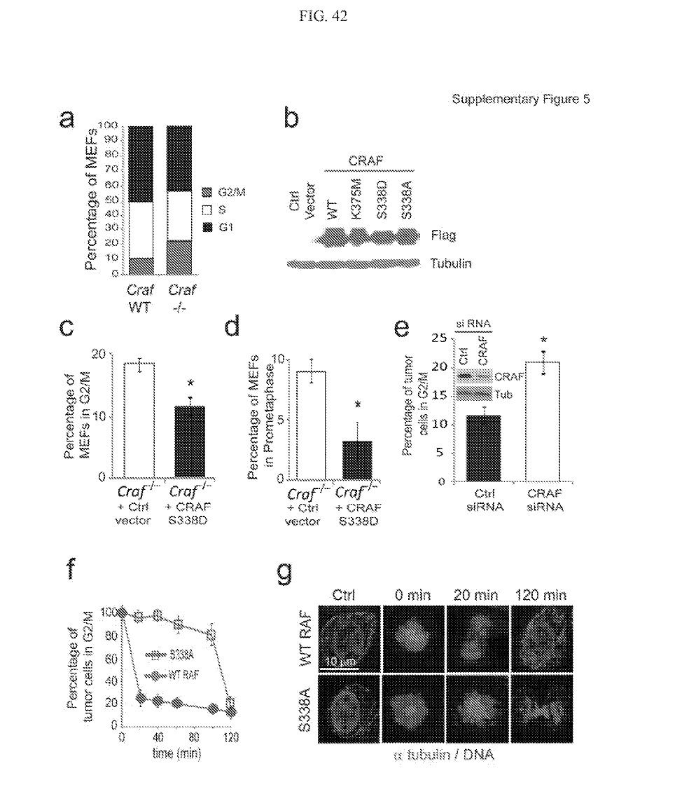

We next examined mouse embryonic fibroblasts (MEFs) derived from c-RAF knockout embryos (CRAF−/−)¹⁷. Asynchronized CRAF−/− MEFs showed a two- and five-fold increase in the number of cells in G2/M and pro-metaphase respectively, compared to wildtype (WT) MEFs (FIG. 34a and FIG. 42a). Following synchronization, CRAF−/− MEFs accumulated in pro-metaphase showing a six-fold delay in mitotic progression compared to WT MEFs (FIGS. 34b, 34c). Depletion of CRAF in tumor cells, similarly led to an accumulation of cells in G2/M (Supplementary FIG. 5e). Importantly, expression of kinase dead (K375M), phospho-mimetic S338D or WT CRAF constructs rescued the mitotic defect of these cells, whereas a non-phosphorylatable (S338A) CRAF mutant, failed to do so (FIG. 34d and FIGS. 42b, 42c, 42d), even though this mutant maintains kinase activity and can dimerize with BRAF (data not shown).

To substantiate these findings, XPA-1 human pancreatic cancer cells expressing WT or the CRAF S338A mutant were synchronized and analyzed for mitotic progression. Cells expressing WT CRAF completed mitosis in twenty minutes whereas cells expressing CRAF S338A showed a six-fold delay in mitosis with cells accumulating in pro-metaphase (FIGS. 42f, 42g). These findings provide genetic evidence to support a kinase-independent role for phospho-5338 CRAF in mitosis.

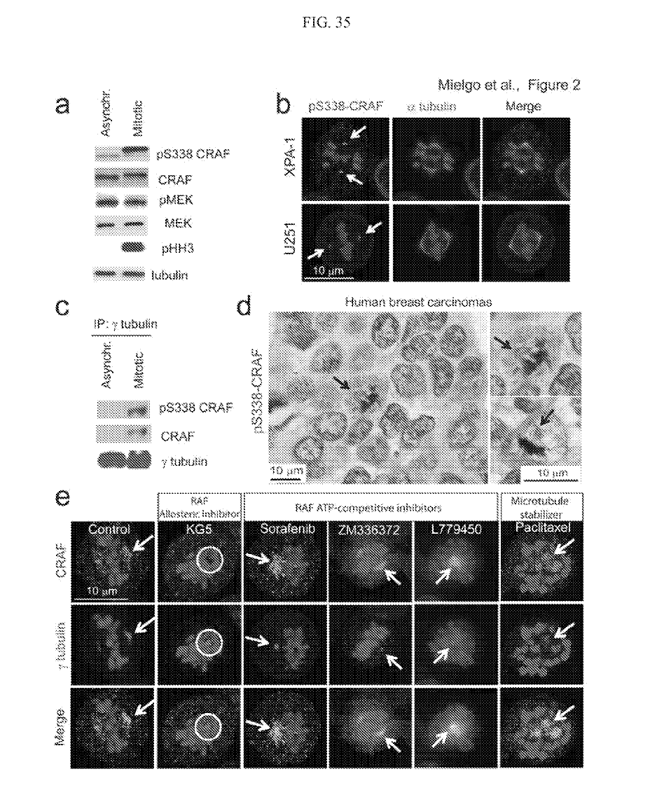

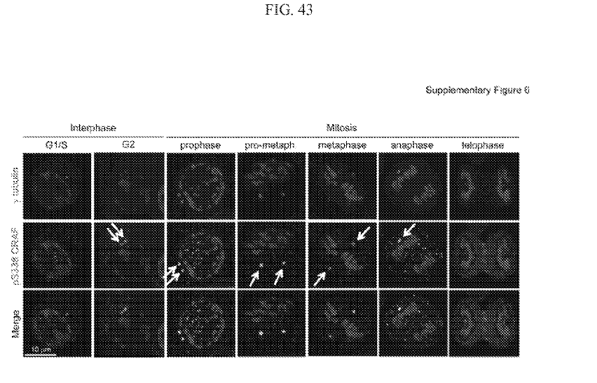

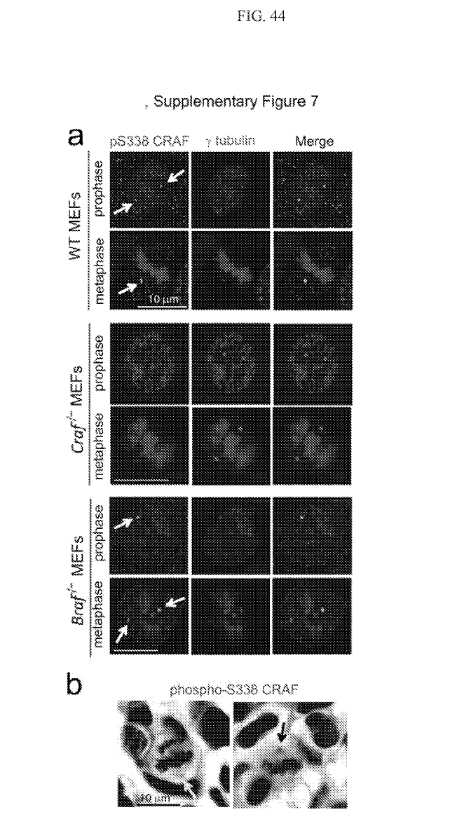

We further investigated the role of CRAF in mitosis and found that phospho-5338 CRAF was strongly increased in mitosis where it co-precipitated with γ-tubulin (FIGS. 35a and 35c) and localized to the centrosomes/mitotic spindle poles of several tumor cell lines in G2/M (FIG. 35b and FIG. 43). We observed similar mitotic localization in biopsies from breast cancer patients (FIG. 35d) and orthotopic breast cancer xenografts (Supplementary FIG. 44b). In fact, the allosteric inhibitor KG5, but not ATP-competitive RAF inhibitors, prevented this localization (FIG. 35e). Furthermore, in cells treated with the microtubule stabilizing agent paclitaxel, CRAF remained localized to the mitotic spindle, suggesting that KG5 and paclitaxel arrest cells at pro-metaphase via distinct molecular mechanisms (FIG. 35e).

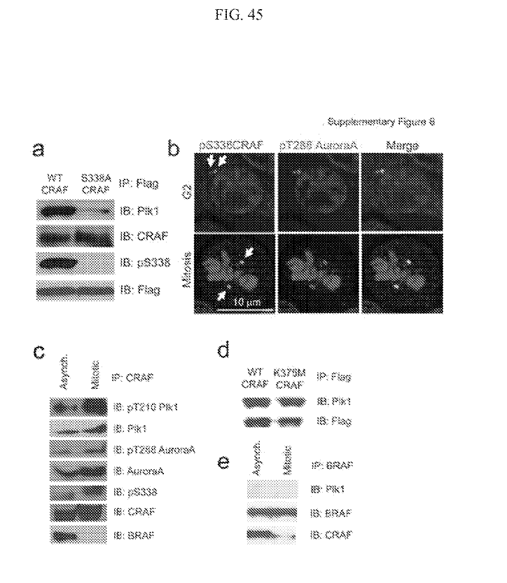

Previous studies have revealed that CRAF and BRAF can form heterodimers¹⁸ and that BRAF can play a role in mitosis¹⁹⁻²³. Therefore, we investigated whether the localization of CRAF to the mitotic spindle was BRAF dependent by analyzing lysates from asynchronized or mitotic cells for BRAF/CRAF heterodimer formation. Interestingly, while BRAF/CRAF heterodimers could readily be detected in asynchronized cells we could not detect such heterodimers in mitotic cells (FIG. 45c). In fact, phospho-S338 CRAF localized to the spindle pole in cells lacking BRAF (FIG. 44a). These results suggest that the role that CRAF plays in mitosis is distinct from that of BRAF.

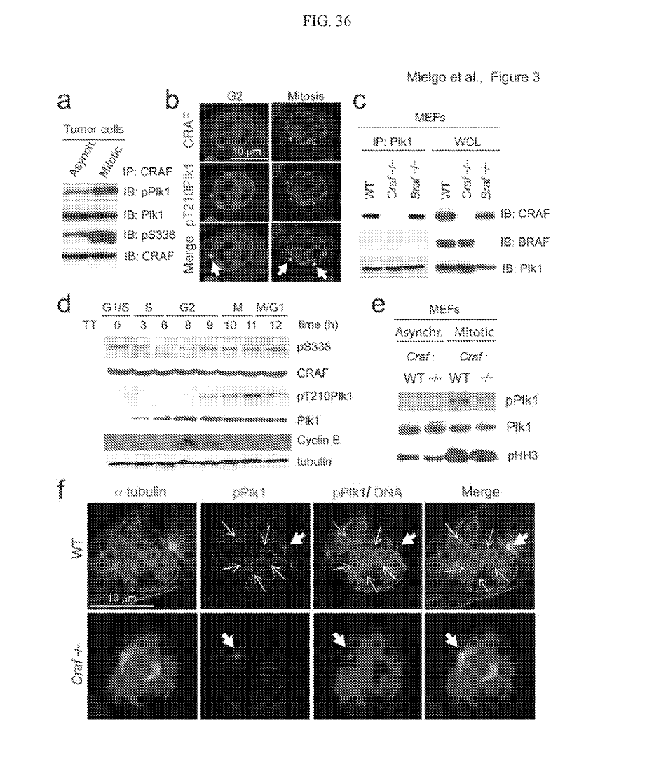

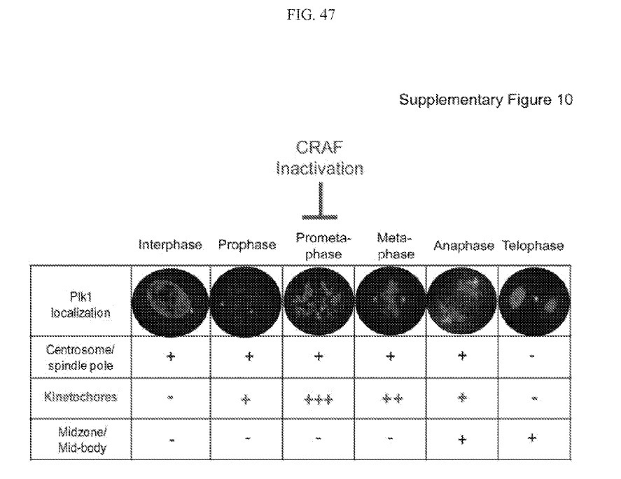

Mitotic progression is regulated by various mitotic kinases such as Aurora kinases, Plk1, and CDK1²⁴⁻²⁶. Plk1 becomes activated by Aurora-A in G2, localizes to centrosomes, spindle poles and accumulates at kinetochores at pro-metaphase to regulate several processes during mitosis²⁷⁻²⁹ (FIG. 47). Accordingly, tumor cells expressing oncogenic RAS are particularly dependent on Plk1 activity for mitotic progression and survival^{30,31}, and inhibition or depletion of Plk1 causes cell cycle delay or arrest at pro-metaphase ^{28,32-35}. To investigate the role of CRAF in tumor cell mitosis, we evaluated whether CRAF could interact with either Aurora-A and/or Plk1. Interestingly, both Aurora-A and Plk1 co-precipitated with CRAF but not BRAF (FIGS. 36a and 36c, and FIGS. 45c and 45e). Moreover, CRAF was co-localized with Aurora-A and Plk1 at the centrosomes in G2 and spindle poles in mitotic tumor cells (FIG. 36b and FIG. 45b). Furthermore, we immunoprecipitated endogenous Plk1 from WT, CRAF−/− and BRAF−/− MEFs and analyzed the immunoprecipitates for C- or BRAF. Interestingly, CRAF, but not BRAF, co-precipitated with Plk1 (FIG. 36c). Moreover, we detected a complex between CRAF and Plk1 in cells expressing WT CRAF and kinase dead (K375M) CRAF (Supplementary FIG. 45d) but this interaction was significantly decreased in cells expressing the S338A RAF mutant (Supplementary FIG. 45a) suggesting that the interaction between CRAF and Plk1 does not depend on CRAF kinase activity but rather requires serine 338 phosphorylation. Thus, CRAF may serve as a scaffold bringing Aurora-A and Plk1 into a functional mitotic complex.

To determine whether the kinetics of CRAF serine 338 phosphorylation during cell cycle progression correlated with Plk1 activation, we analyzed CRAF (S338) and Plk1 (T210) phosphorylation throughout the cell cycle from G1-M. Interestingly, phospho-S338 CRAF levels were increased in G1, as expected, but showed a second wave of phosphorylation at G2/M beginning immediately prior to Plk1 phosphorylation (FIG. 36d), consistent with the notion that CRAF phosphorylation on serine 338 precedes Plk1 activation.

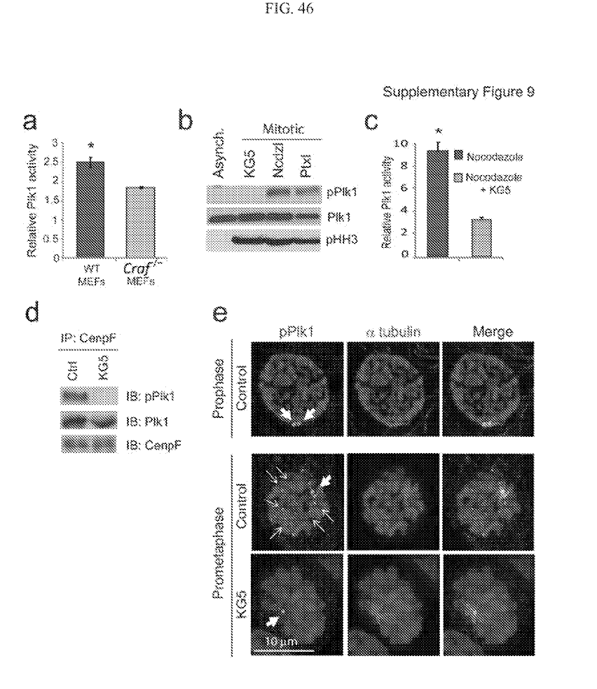

To further explore a role for CRAF in Plk1 activation, lysates from WT and CRAF−/− MEFs were subjected to immunoblotting for phospho-Plk1 or evaluated for Plk1 enzymatic activity (FIG. 36e and FIG. 46a). Indeed, CRAF−/− MEFs demonstrated minimal Plk1 activity compared to WT MEFs. Furthermore, tumor cells arrested in pro-metaphase with KG5 showed decreased Plk1 activity compared to cells arrested with nocodazole or paclitaxel (FIGS. 46b, 46c). While treatment of cells with KG5 had no effect on Plk1 localization to the spindle pole, it prevented the subsequent accumulation of active Plk1 at the kinetochores (FIGS. 46d, 46e) and similar findings were observed in CRAF deficient MEFs (FIG. 36f). These findings were substantiated since an active form of Plk1 was able to rescue the G2/M delay caused by CRAF depletion (FIG. 11). Together, these results indicate that CRAF potentiates Plk1 and/or Aurora-A activation leading to accumulation of active Plk1 at the kinetochores. This process facilitates mitotic progression through pro-metaphase and can be blocked by inhibition of phospho-S338 CRAF via allosteric RAF blockade.

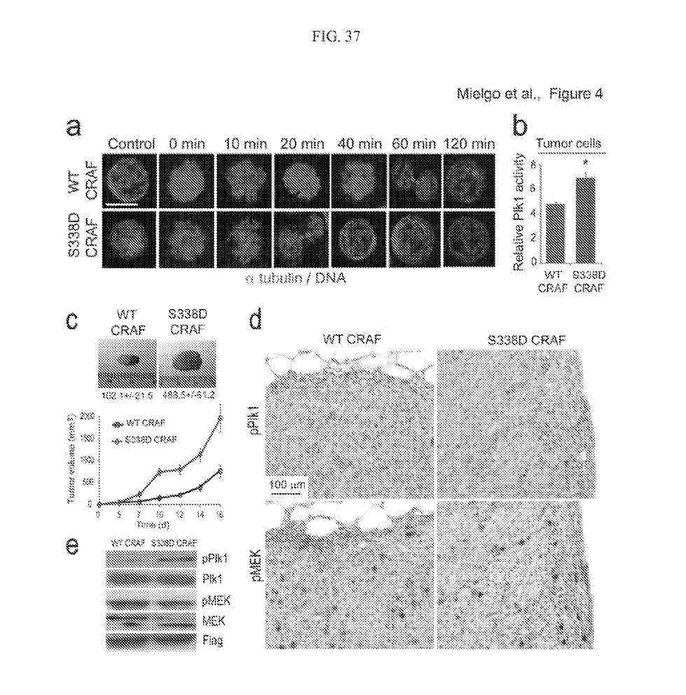

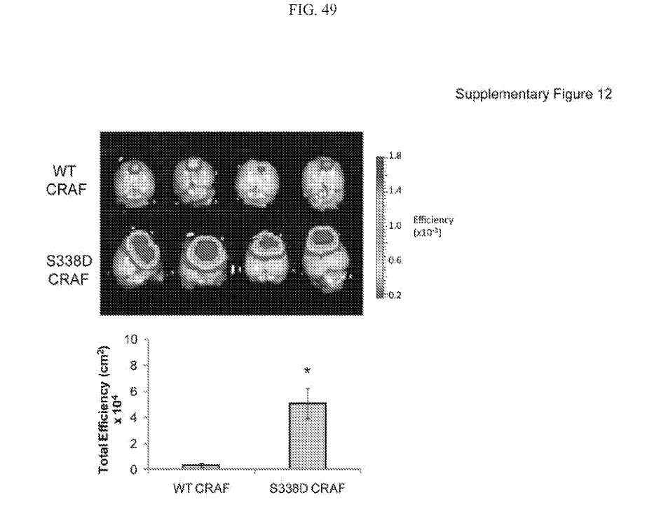

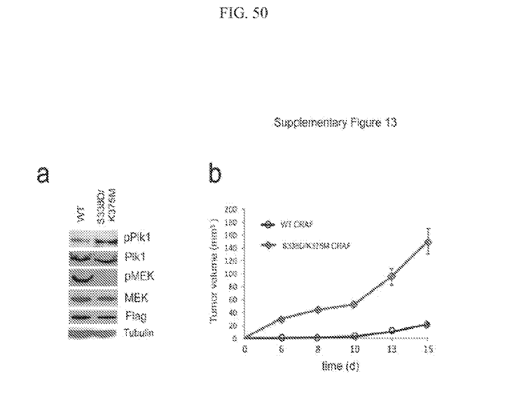

To investigate the impact of the CRAF/Plk1 signaling module in tumor progression, we evaluated the tumor growth capacity of human colon carcinoma and glioblastoma cells stably expressing the phospho-mimetic S338D CRAF mutant. HCT-116 colon cells expressing the S338D CRAF mutant showed accelerated mitosis and a five-fold increase in tumor growth relative to cells expressing WT CRAF and this was associated with increased phospho-Plk1 but not phospho-MEK expression in these tumors (FIGS. 37a, 37b, 37c, 37d, 37e). Similarly, U-87 glioblastoma cells expressing S338D CRAF produced a 15-fold increase in brain tumor growth relative to cells expressing WT CRAF (FIG. 49). Furthermore, cells expressing a double mutant S338D kinase dead (S338D/K375M) also showed increase Plk1 activity and tumor growth in mice but failed to activate MEK (FIG. 50). These results demonstrate that phospho-5338 CRAF is an important mediator of tumor progression based on its capacity to promote mitosis in a manner that is independent of active MEK.

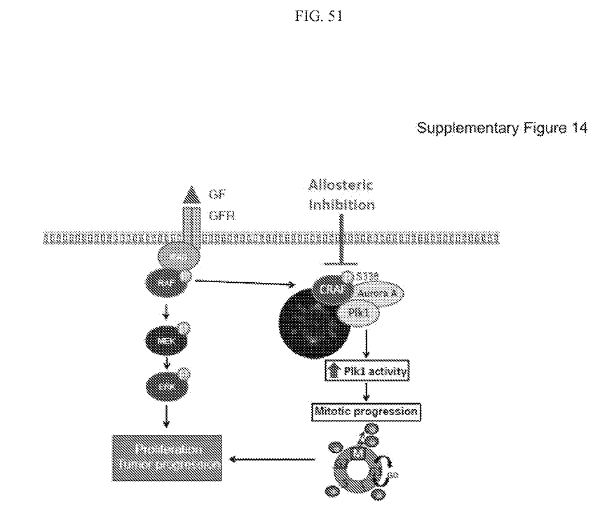

While RAF is an essential component of the canonical MAPK signaling pathway various reports reveal that RAF exerts kinase independent functions^{17,38-40}. We previously showed that CRAF, independent of its kinase activity, could translocate to the mitochondria and protect cells from apoptosis by inhibiting the pro-apoptotic protein ASK1^{14,15}. Here, we reveal a kinase independent function of CRAF in cell proliferation and demonstrate that phospho-5338 CRAF localizes to centrosomes/mitotic spindle poles where it interacts with Aurora-A and Plk1, promotes Plk1 activation and thereby mitotic and tumor progression (FIG. 51).

Whether CRAF interaction with Aurora-A and Plk1 is direct or indirect still needs to be determined. However, these data support the conclusion that CRAF may act as an adaptor protein to promote Aurora A and/or Plk1 activation facilitating mitosis and tumor progression. While our findings may be relevant to all cells undergoing mitosis, targeting this pathway with allosteric RAF inhibitors, like KG5, might be particularly effective during angiogenesis^{12,} and tumor growth, processes characterized by highly proliferative cells. Finally, our studies reveal that allosteric inhibitors designed to block phospho-5338 CRAF and its function in mitosis represent a new therapeutic approach to inhibit the growth of a wide range of cancers.

Figure legends

FIG. 34. CRAF is required for mitotic progression: (a) Cell cycle analysis of WT and CRAF−/− MEFs. Left graph, Cells in G2/M were quantified by flow cytometry. Right graph, Cells at pro-metaphase were quantified using confocal microscopy. Error bars represent s.d. (n=4); * two tailed p value=0.0079 (left graph) and p=0.0055 (right graph) using a Mann Whitney U test. (b) WT and CRAF−/− MEFs were synchronized at pro-metaphase with a thymidine-nocodazole block as described in these Methods, and subsequently released from the blockade and allowed to progress through mitosis. Quantification of cells in G2/M was performed by flow cytometry. Error bars represent s.d. (n=3). (c) Confocal microscopy images of WT and CRAF−/− cells progressing through mitosis at 0, 60 and 360 minutes after release from pro-metaphase blockade. Cells were stained for α-tubulin (in red) and DNA (TOPRO-3™ in blue). Scale bar, 10 μm. (d) WT and CRAF−/− MEFs were transfected with vector control, WT CRAF, kinase dead (K375M) CRAF, phospho-mimetic (S338D) CRAF or non-phosphorylatable (S338A) CRAF mutants. Cell cycle analysis and quantification of cells in G2/M was performed by flow cytometry. Error bars represent s.d. (n=3); * two tailed p value=0.0084 using a Mann Whitney U test. (e) Immunohistochemical staining of phospho-5338 CRAF and phospho-histone H3 (mitotic marker) in orthotopic breast and tumor xenografts untreated or treated systemically with KG5 (50 mg/Kg for 3 days Scale bar, 50 μm. Scale bar, 50 μm. Circles indicate cells in pro-metaphase. Right, Quantification of cells in pro-metaphase. Error bars represent s.d. (n=12); * two tailed p value=0.006 using Student T test.

FIG. 35. Phospho-serine 338 CRAF is upregulated in mitosis and localizes to mitotic spindles in human cell lines and tumor biopsies: FIG. 35(a) Immunoblot analysis of human colon carcinoma HCT-116 cells asynchronized and synchronized at pro-metaphase. pS338 refers to phospho-5338 CRAF, pMEK refers to phospho-MEK and pHH3 refers to phospho-histone H3. Data are representative of three independent experiments. FIG. 35 (b) Confocal microscopy images of human pancreatic XPA-1 and glioblastoma U251 cells during mitosis, stained for phospho-5338 CRAF (in green), α-tubulin (in red) and DNA (TOPRO-3™ in blue). Scale bar, 10 μm. White arrows indicate localization of phospho-S338 CRAF at the mitotic spindle. FIG. 35 (c) Immunoblot analysis of γ-tubulin immunoprecipitates from human colon carcinoma HCT-116 cells asynchronized and synchronized at pro-metaphase. Data are representative of three independent experiments. FIG. 35(d) Immunohistochemical staining of phospho-S338 CRAF in tumor biopsies from breast cancer patients. Scale bar, 10 μm. FIG. 35(e) Confocal microscopy images of XPA-1 cells treated with KG5, sorafenib, ZM336372, L779450 or paclitaxel and stained for CRAF (in green), γ-tubulin (in red) and DNA (TOPRO-3™ in blue). Scale bar, 10 μm. White arrows indicate localization of CRAF at the spindle pole. White circle indicates the absence of CRAF at the spindle pole.

FIG. 36. CRAF interacts with Plk1 and promotes its activation and accumulation to the kinetochores at pro-metaphase: FIG. 36(a) Immunoblot analysis of CRAF immunoprecipitates from HCT-116 asynchronized and synchronized at pro-metaphase cells. Data are representative of three independent experiments. FIG. 36(b) Confocal microscopy images of HCT-116 cells synchronized at G2 and pro-metaphase (as described in Methods) and stained for CRAF (in green), phospho-T210 Plk1 (in red) and DNA (TOPRO-3™ in blue). White arrows indicate co-localization of CRAF with phospho-Plk1 at the centrosomes and mitotic spindle poles. FIG. 36(c) Immunoblot analysis of Plk1 immunoprecipitates from WT, CRAF−/− and BRAF−/− MEFs. Data are representative of three independent experiments. FIG. 36(d) Immunoblot analysis from G1-M, of phospho-S338 CRAF, total CRAF, phospho-T210 Plk1, total Plk1, cyclin B and tubulin of HCT-116 cells synchronized at the G1/S boundary. Cells were synchronized at the G1/S boundary by a double thymidine block as described in Methods. Data are representative of two independent experiments. FIG. 36(e) Immunoblot analysis of asynchronous and mitotic WT and CRAF−/− MEFs. FIG. 36(f) Confocal microscopy images of WT and CRAF−/− MEFs at pro-metaphase. Cells were stained for a tubulin (in green), phospho-T210 Plk1 (in red) and DNA (TOPRO-3™ in blue). Thick white arrows indicate localization of phospho-Plk1 at the mitotic spindle pole and narrow white arrows indicate localization of phospho-Plk1 at the kinetochores. Scale bar, 10 p.m.

FIG. 37. Phospho-mimetic CRAF S338D mutation drives tumor growth and activates Plk1 in vivo: FIG. 37(a) HCT-116 human colon carcinoma cells ectopically expressing either WT RAF or S338D mutant CRAF were arrested in pro-metaphase as described in Methods, and subsequently allowed to progress through mitosis. Cells were stained for α-tubulin (in red) and DNA (TOPRO-3™ in blue) at 0, 10, 20, 40, 60 and 120 minutes after release from pro-metaphase blockade and mitotic progression was analyzed by confocal microscopy. Scale bar, 10 p.m. Data are representative of three independent experiments. FIG. 37(b) Plk1 kinase activity assay performed in HCT-116 cells expressing WT CRAF or a phospho-mimetic S338D CRAF mutant. Error bars represent s.d. (n=3); * two tailed p value=0.011 using a Mann Whitney U test. FIG. 37(c) HCT-116 cells expressing WT or S338D CRAF Flag tagged were injected subcutaneously in the flank of immune-compromised nude mice. Tumor images, average weights +/−s.e and tumor size measurements are shown (n=20). FIG. 37(d) Immunohistochemical staining of phospho-Plk1 and phospho-MEK in mouse tissues from tumors expressing WT or S338D CRAF. Scale bar, 100 μm. FIG. 37(e) Immunoblot analysis of phospho-T210 Plk1, Plk1, phospho-MEK, MEK and Flag from tumor lysates from HCT-116 cells expressing WT or S338D CRAF. Data are representative of five independent experiments.

FIG. 38. Allosteric inhibition of RAF arrests cells at pro-metaphase in a way that is MEK-independent but requires CRAF phosphorylation on serine 338. FIG. 38(a) Bright field images (scale bar, 20 μm), cell cycle analysis and confocal microscopy images (scale bar, 10 μm, laminB in red and DNA in blue) of XPA-1 cells treated for 20 hours with the allosteric RAF inhibitor KG5, the ATP-competitive inhibitor Sorafenib, the MEK inhibitor PD0325901, the control compound KG1 at 5 μM or paclitaxel at 200 nM. Cells were harvested after 20 hours and the cell cycle stage was determined by flow cytometry and confocal microscopy. White arrows indicate cells arrested at pro-metaphase. Scale bars, 10 μm. FIG. 38(b) Representative confocal microscopy images of untreated XPA-1 tumor cells undergoing mitosis (left image) and XPA-1 cell arrested with KG5 at pro-metaphase (right image). Cells were stained with α-tubulin in red and DNA in blue. Scale bar, 10 μm. FIG. 38(c) XPA-1 cells transfected with a vector control or active MEK were treated with 1 μM KG5 for 20 hours or left untreated and the G2/M population was quantified by flow cytometry. Error bars represent s.d. (n=3); * two tailed p value=0.00063 using a Mann Whitney U test. FIG. 38(d) Immunoblot analysis of XPA-1 cells untreated or treated with 1 μM KG5 for 18 hours. Data are representative of four independent experiments. FIG. 38(e) HCT-116 cells stably expressing WT or the phospho-mimetic S338D CRAF construct were treated with 1 μM KG5 for 18 hours and cells arrested in G2/M were quantified by flow cytometry. Error bars represent s.d. (n=3); * two tailed p value=0.010 using a Mann Whitney U test. Inset, Immunoblot analysis of HCT-116 cells untreated or treated with 1 μM KG5 for 18 hours.

FIG. 39. Allosteric inhibition of RAF with KG5, unlike ATP-competitive RAF inhibitors, induces a G2/M arrest followed by cell death. FIG. 39 graphically illustrates cell cycle analysis of XPA-1 cells treated with the allosteric RAF inhibitor KG5, the microtubule stabilizer paclitaxel, or the ATP-competitive RAF inhibitors sorafenib, L-779450, GW 5074, and ZM-336372. Cells were harvested after 6, 18, 24, 48 and 72 hours and the percentages of cells in G2/M and SubG1 were determined by flow cytometry. Data are representative of three independent experiments.

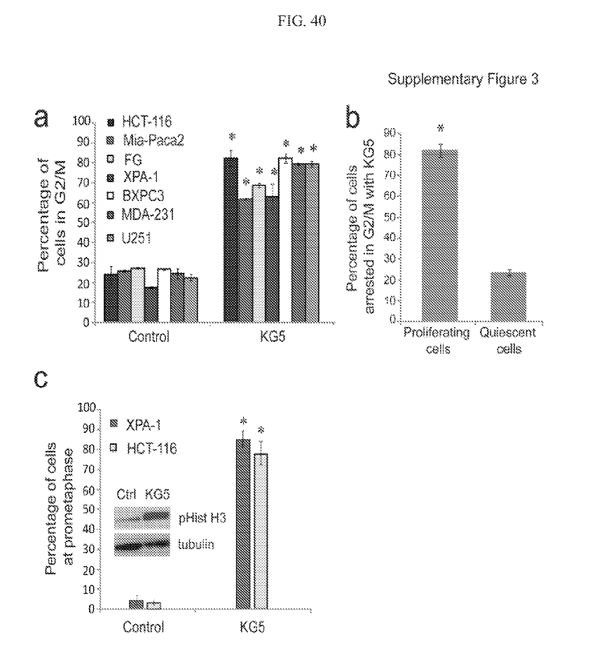

FIG. 40. Allosteric inhibition of RAF arrests proliferating cells at pro-metaphase. FIG. 40(a) Cell cycle analysis and G2/M quantification of human colon (HCT-116), pancreatic (Mia-Paca2, FG, XPA-1, BXPC3), breast (MDA-MB-231) and brain (U251) cancer cell lines untreated or treated for 20 hours with 5 μM of KG5. Error bars represent s.d. (n=4); * two tailed p value=0.0023 using Mann Whitney U test. FIG. 40(b) HCT-116 cells were cultured under low (30%) or high (90%) confluency and treated with 1 μM KG5 for 20 hours. Quantification of cells arrested in G2/M was performed by flow cytometry. Error bars represent s.d. (n=3); * two tailed p value=0.0021 using Mann Whitney U test. FIG. 40(c) HCT-116 and XPA-1 cells were treated for 20 hours with 1 μM of KG5, stained for α-tubulin/DNA, and cells arrested in pro-metaphase were quantified by confocal microscopy. Error bars represent s.d. (n=3); * two tailed p value=0.0008 using Mann Whitney U test. Lysates from the same cells were also analyzed by immunoblotting for the expression of the mitotic marker phospho-Histone H3 (inset). Data are representative of three independent experiments.

FIG. 41. Phospho-5338 CRAF is highly expressed in tumors and can be blocked by an allosteric RAF inhibitor: FIG. 41(a) Immunohistochemical staining of phospho-S338 CRAF in normal human brain and human glioblastoma tissues. Scale bar, 100 μm. FIG. 41(b) Immunohistochemical staining of phospho-S338 CRAF in orthotopic brain tumor xenografts untreated or treated systemically with KG5 (50 mg/Kg) for 3 days. Scale bar, 50 p.m. FIG. 42. Phospho-5338 CRAF promotes mitosis: FIG. 42(a) Quantification of WT and CRAF−/− cells in the G1, S and G2/M phases of the cell cycle. Data are representative of three independent experiments. FIG. 42(b) Immunoblot analysis of CRAF−/− MEFs transfected with WT, kinase dead (K375M), phospho-mimetic (S338D) and non-phosphorylatable (S338A) FLAG tagged CRAF constructs. FIG. 42(c) Cell cycle analysis of CRAF−/− MEFs transfected with vector control or S338D CRAF was performed. Cells in G2/M were quantified by flow cytometry. Error bars represent s.d. (n=3); * two tailed p value=0.0067 using a Mann Whitney U test. FIG. 42(d) Cells at pro-metaphase were quantified using confocal microscopy. Error bars represent s.d. (n=3); * two tailed p value=0.0048 using a Mann Whitney U test. FIG. 42(e) HCT-116 cells were transfected with control or CRAF siRNAs and cells in G2/M were quantified by flow cytometry. Error bars represent s.d. (n=3); * two tailed p value=0.0062 using a Mann Whitney U test. Lysates of these cells were analyzed for CRAF expression by immunoblotting (inset). FIG. 42(f) XPA-1 cells ectopically expressing either WT RAF or S338A mutant CRAF were arrested in pro-metaphase as described in Methods, and subsequently allowed to progress through mitosis. Quantification of cells in G2/M was performed by flow cytometry. Error bars represent s.d. (n=3). FIG. 42(g) Confocal microscopy images of cells progressing through mitosis at 0, 20, 100 and 120 minutes after release from pro-metaphase blockade. Cells were stained for a tubulin (in red) and DNA (TOPRO-3™ in blue). Scale bar, 10 p.m.

FIG. 43: Phospho-5338 CRAF localizes to centrosomes in G2 phase and spindle poles during tumor cell mitosis: the figure illustrates confocal microscopy images of human HCT-116 cells synchronized at the G1/S boundary by a double thymidine block (as described in Methods) and stained for γ tubulin (in red), phospho-5338 CRAF (in green), and DNA (TOPRO-3™ in blue). Scale bar, 10 p.m. White arrows indicate localization of phospho-5338 CRAF at the centrosomes in G2 and the mitotic spindle poles from pro-metaphase to anaphase.

FIG. 44. Phospho-5338 CRAF localizes to mitotic spindle poles in a BRAF-independent manner: FIG. 44(a) Confocal microscopy images of WT, CRAF−/− and BRAF−/− MEFs at prophase and metaphase stained for phospho-5338 CRAF (in green), γ-tubulin (in red) and DNA (TOPRO-3™ in blue). Scale bar, 10 p.m. White arrows indicate localization of phospho-5338 CRAF at the mitotic spindle poles. FIG. 44(b) Immunohistochemical staining of phospho-5338 CRAF in tumor biopsies from mouse orthotopic breast tumour xenografts. Scale bar, 10 p.m.

FIG. 45. CRAF interacts with Plk1 and Aurora A and this interaction does not require CRAF kinase activity but requires serine 338 phosphorylation. FIG. 45(a)

Immunoblot analysis of Flag immunoprecipitates from XPA-1 cells expressing Flag-tagged WT or S338A CRAF. Data are representative of three independent experiments. FIG. 45(b) Confocal microscopy images of a HCT-116 synchronized in G2 or mitosis (as described in methods) and stained for phospho-5338 CRAF (in green), phospho-T288 Aurora A (in red) and DNA (in blue). White arrows indicate colocalization between phospho-5338 CRAF and phospho-T288 Aurora A. Scale bar, 10 p.m. FIG. 45(c) Immunoblot analysis of CRAF immunoprecipitates from XPA-1 cells asynchronized or synchronized in mitosis. Data are representative of three independent experiments. FIG. 45(d) Immunoblot analysis of Flag immunoprecipitates from XPA-1 cells expressing Flag-tagged WT or kinase dead (K375M) CRAF. Data are representative of three independent experiments. FIG. 45(e) Immunoblot analysis of BRAF immunoprecipitates from HCT-116 cells asynchronized or synchronized in mitosis.

FIG. 46. CRAF regulates Plk1 activity and localization. FIG. 46(a) Plk1 kinase activity assay performed in WT and CRAF−/− MEFs. Error bars represent s.d. (n=3); * two tailed p value=0.029 using Mann Whitney U test. FIG. 46(b) Immunoblot analysis of XPA-1 cells asynchronous or arrested in mitosis by treatment with KG5, nocodazole or paclitaxel (referred to as Ncdzl and Ptxl respectively). pPlk1 refers to phospho-T210 Plk1 and pHH3 refers to phospho-Histone H3. Data are representative of three independent experiments. FIG. 46(c) Plk1 kinase activity assay performed in HCT-116 cells treated with nocodazole or nocodazole and KG5. Error bars represent s.d. (n=2); * two tailed p value=0.002 using Mann Whitney U test. FIG. 46(d) Immunoblot analysis of CenpF immunoprecipitates from HCT-116 cells untreated or treated with KG5. Data are representative of three independent experiments. FIG. 46(e) Confocal microscopy images of XPA-1 cell at prophase (upper panel), pro-metaphase control untreated (middle panel) or treated with KG5 (lower panel). Cells were stained for phospho-T210 Plk1 (in green), a tubulin (in red) and DNA (TOPRO-3™ in blue). Thick white arrows indicate localization of pPlk1 at the mitotic spindle pole and narrow white arrows indicate localization of pPlk1 at the kinetochores. Scale bar, 10 p.m.

FIG. 47 illustrates data showing that allosteric inhibition or depletion of CRAF prevents active Plk1 accumulation at the kinetochores in pro-metaphase; the table indicates: Plk1 localization before mitosis and during the different stages of mitosis (from prophase to telophase). Phospho-T210 Plk1 is stained in green and DNA in blue.

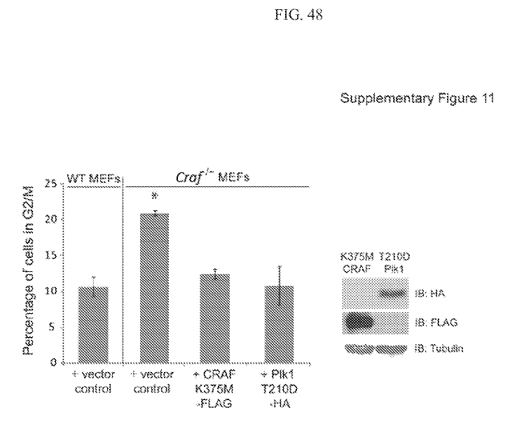

FIG. 48. Kinase dead CRAF and constitutively active T210D Plk1 are both capable of rescuing the CRAF−/− MEFs phenotype. FIG. 48(a) CRAF−/− MEFs were transfected with vector control, kinase dead (K375M) CRAF or a constitutively active T210D Plk1 mutant and the G2/M population was quantified by flow cytometry. Error bars represent s.d. (n=3); * two tailed p value=0.0055 using a Mann Whitney U test. FIG. 48(b) Immunoblot analysis of CRAF−/− MEFs transfected with kinase dead (K375M) CRAF FLAG tagged or a constitutively active T210D Plk1 HA-tagged mutant.

FIG. 49. Phospho-mimetic CRAF S338D mutation drives tumor growth in a brain orthotopic model. FIG. 49 illustrates staining of RFP-labeled U-87 human glioblastoma cells expressing WT or S338D CRAF, which were orthotopically implanted in the brain of immune-compromised nude mice; fluorescence images were recorded and tumor sizes analyzed using IVIS LIVINGIMAGET™ software; and a graphic illustration summarizing the results; values are shown as average fluorescence efficiency +/−SD. (n=9); * two tailed p value=0.003 using Student T test.

FIG. 50. Phospho-mimetic kinase dead CRAF S338D K375M mutation drives tumor growth. FIG. 50(a) Immunoblot analysis of phospho-T210 Plk1, Plk1, phospho-MEK, MEK, Flag and tubulin from U-87 cells expressing WT or S338D/K375M CRAF. Data are representative of five independent experiments. FIG. 50(b) U-87 cells expressing WT or S338D/K375M CRAF were injected subcutaneously in the flank of immune-compromised nude mice. Tumor size measurements are shown (n=10).

FIG. 51 illustrates a model depicting the MEK-independent role of CRAF in mitosis and tumor progression. Phospho-5338 CRAF localizes to centrosomes in G2 and mitotic spindle poles in mitosis where it interacts with Plk1 and Aurora A and promotes Plk1 activation, leading to mitosis and tumor progression. Allosteric inhibition of CRAF prevents phosphorylation of CRAF at serine 338, and thereby activation of Plk1, causing cells to arrest at pro-metaphase.

### Methods

Cell Culture and Transfections

Human pancreatic (FG, XPA-1, MiaPaca-2), breast (MDA-MB-231), colon (HCT-116) and brain (U251) cancer cell lines as well as mouse embryonic fibroblasts (MEFs) were cultured in DMEM supplemented with 10% fetal bovine serum, glutamine and non-essential amino acids. XPA-1 and HCT-116 cells were transiently transfected with WT and S338A CRAF-Flag and CRAF-GFP constructs using the Lipofectamine reagent (Invitrogen) following manufacturer's protocol or stably transfected using a lentiviral system. MEFs were transiently transfected with S338D CRAF-Flag or control vector using the AMAXA™ system following manufacturer's protocol. CRAF expression was confirmed by immunoblotting with anti-flag antibody (Sigma) or by GFP fluorescence microscopy.

Cell Cycle Analysis and Drug Treatments

Cell cycle analysis was performed by flow cytometry following propidium iodide staining. Briefly, cells were harvested, fixed with ice cold methanol, treated for 45 min with 10 μg/ml of RNase and resuspended in PBS containing 10 μg/ml of PI, and analyzed by flow cytometry. Cell cycle analysis was performed in cells treated for 18 hours with the allosteric RAF inhibitor KG5, the ATP-competitive RAF inhibitors: sorafenib, L-779450, GW5074, and ZM-336372, the MEK inhibitor (PD0325901) at 1 M concentration or paclitaxel at 100 nM.

Cell Synchronization

To analyze mitotic progression, CRAF WT and CRAF−/− MEFs and HCT-116 cells expressing WT or S338D CRAF were synchronized at pro-metaphase by a thymidine-nocodazole arrest as described²⁹. Cells synchronized at pro-metaphase (approximately 15% for the MEFs and approximately 90% for the HCT-116) were collected by shake-off. Medium was replaced with nocodazole-free medium to allow cells to re-enter the cell cycle and progress through mitosis. The time required to accomplish mitosis was assessed by flow cytometry and confocal microscopy. To analyze the kinetics of CRAF phosphorylation on serine 338 and Plk1 phosphorylation on threonine 210, as well as phospho-5338 CRAF localization at the different stages of the cell cycle, HCT-116 cells were synchronized at the G1-S boundary by a double-thymidine treatment as described²⁹.

Immunofluorescence Analysis

Cells attached to cover-slips were fixed with cold methanol and permeabilized in PBS containing 0.1% triton for two minutes and blocked for 60 minutes, at room temperature with 2% BSA in PBS. Cells were stained with antibodies to CRAF (Abcam), phospho-serine 338 CRAF (SantaCruz), phospho-serine 338 CRAF (Thermo Scientific), a tubulin (Calbiochem), γ tubulin (Abcam), laminB (SantaCruz), Plk1 (Abcam), phospho-T210 Plk1 (Abcam), or phospho-T288 Aurora A (cell signaling). All primary antibodies were used at 1:100 dilution, for two hours at room temperature. After washing several times with PBS, cells were stained for two hours at room temperature, with secondary antibody specific for mouse, rabbit or goat (Invitrogen), as appropriate, diluted 1:300 and co-incubated with the DNA dye TOPRO-3™ (1:500) (Invitrogen). Samples were mounted in VECTASHIELD™ hard-set mounting media (Vector Laboratories) and imaged on a Nikon ECLIPSE C1™ confocal microscope with a 1.4 NA 60× oil-immersion lens, using minimum pinhole (30 μm). Images were captured using EZ-C_{1-3.50} imaging software.

Kinase Assay

Plk1 enzymatic activity in cell lysates from WT, CRAF−/− MEFs, HCT-116 cells expressing WT or S338D CRAF and HCT-116 cells treated with the allosteric RAF inhibitor KG5 was determined using the Plk1 kinase kit from MBL and following the manufacturer's protocol.

Orthotopic Breast Cancer Xenograft Model

Tumors were generated by injection of MDA-MB-231 human breast carcinoma cells (2×10⁶ tumor cells in 50 μl of sterile PBS) into the mammary fatpad of 6-8 week old female nude mice. Tumors were established for 2 weeks before beginning dosing. Mice were dosed with vehicle (10% HS-15) or 50 mg/kg KG5 (i.p. bid) for 3 days prior to harvest. All research was conducted under protocols approved by the UCSD animal subjects committee and in accordance with the guidelines set forth in the NIH Guide for the Care and Use of Laboratory Animals.

Orthotopic Brain Cancer Xenograft Model

Tumors were generated by injection of RFP-labeled U-87 human glioblastoma cells (10⁵ tumorcells in 2 μl of sterile PBS) into the brain of 6-8 week old female nude mice. Tumors were established for 3 weeks before beginning dosing. Mice were dosed with vehicle (10% HS-15) or 50 mg/kg KG5 (i.p. bid) for 3 days prior to harvest. Alternatively, U-87 cells stably expressing WT CRAF or the phospho-mimetic S338D CRAF mutant were injected into the brain of 6-8 week old female nude mice. Brains were harvested at day 15 and fluorescence images recorded using the IVIS-200™ imaging system (Xenogen Corp., Hopkinton, Mass.). Tumor sizes were measured as a function of fluorescence intensity using the ROI function of the IVIS Living Image Software.

Subcutaneous Mouse Tumor Model

HCT-116 human colon carcinoma cells stably expressing WT or S338D CRAF were injected subcutaneously (2×10⁶ tumor cells in 50 μl of sterile PBS), in the left or right flank, respectively, of 6-8 week old female nude mice. Tumors were measured every 2-3 days with calipers and harvested, weighed and processed at day 16.

Immunohistochemical Analysis

Immunostaining was performed according to the manufacturer's recommendations (Vector Labs), on 5 μM sections of paraffin-embedded tumors from the orthotopic xenograft breast and brain cancer mouse models, the subcutaneous tumor mouse model or from human patients diagnosed with breast or brain cancer (as approved by the Institutional Review Board at University of California, San Diego). Antigen retrieval was performed in citrate buffer pH 6.0 at 95° C. for 20 min. Sections were treated with 0.3% H₂O₂ for 30 min, blocked in normal goat serum, PBS-T for 30 min followed by Avidin-D and then incubated overnight at 4° C. with primary antibodies against phospho-S338 CRAF (Thermo Scientific), phospho-T210 Plk1 (Rockland) and phospho-Histone H3 (Cell signaling) diluted 1:100 in blocking solution. Tissue sections were washed and then incubated with biotinylated secondary antibody (1:500, Jackson ImmunoResearch) in blocking solution for 1 h. Sections were washed and incubated with VECSTATIN ABC™ (Vector Labs) for 30 min. Staining was developed using a Nickel-enhanced diamino-benzidine reaction (Vector Labs) and sections were counter-stained with hematoxylin.

Immunoprecipitation and Immunoblot Analysis Cells were lysed in either NP40 lysis buffer (150 mM NaCl, 50 mM Tris Base pH 7.4, 1% NP40) or RIPA buffer (50 mM Tris pH 7.4, 100 mM NaCl, 0.1% SDS) supplemented with complete protease inhibitor mixture (Roche), 50 mM NaF and 1 mM Na₃VO₄ and centrifuged at 13,000×g for 10 min at 4° C. Protein concentration was determined by BCA assay. 500 ng of protein were immunoprecipitated with 2 ng of anti-Flag (Sigma), anti-CRAF (BD Pharmingen), anti-Plk1 (Abcam), anti-γ-Tubulin (sigma) or anti-cenpF (Santa Cruz) antibodies overnight at 4° C. followed by capture with 25 μl of protein A/G (Pierce). Beads were washed five times, eluted in boiling Laemmli buffer, resolved on 10% SDS-PAGE and immunoblotting was performed with anti-Plk1 antibody (Abcam), anti-CRAF antibody (BD Pharmingen), anti-Aurora A (Abcam), anti-phospho-S338 CRAF (Cell Signaling), anti-phospho-T210 Plk1 or anti-phospho-T288 Aurora A (Cell signaling) diluted 1:1000. For immunoblot analysis, 30 ng of protein was boiled in

Laemmli buffer and resolved on 10% gel.

Statistical Analysis

All statistical analysis were performed with Prism GRAPHPAD™. Two-tailed Mann-Whitney U test or Student T-test was used to calculate statistical significance. A P value <0.05 was considered to be significant.

### REFERENCES

1. Wellbrock, C., Karasarides, M. & Marais, R. The RAF proteins take centre stage. Nature reviews 5, 875-885 (2004).
2. Dhillon, A. S., Hagan, S., Rath, O. & Kolch, W. MAP kinase signalling pathways in cancer. Oncogene 26, 3279-3290 (2007).
3. Bollag, G., et al. Clinical efficacy of a RAF inhibitor needs broad target blockade in BRAF-mutant melanoma. Nature 467, 596-599 (2010).
4. Flaherty, K. T., et al Inhibition of mutated, activated BRAF in metastatic melanoma. The New England journal of medicine 363, 809-819 (2010).
5. Joseph, E. W., et al. The RAF inhibitor PLX4032 inhibits ERK signaling and tumor cell proliferation in a V600E BRAF-selective manner. Proceedings of the National Academy of Sciences of the United States of America 107, 14903-14908 (2010).
6. King, A. J., et al. Demonstration of a genetic therapeutic index for tumors expressing oncogenic BRAF by the kinase inhibitor SB-590885. Cancer research 66, 11100-11105 (2006).
7. Tsai, J., et al. Discovery of a selective inhibitor of oncogenic B-Raf kinase with potent antimelanoma activity. Proceedings of the National Academy of Sciences of the United States of America 105, 3041-3046 (2008).
8. Yang, H., et al. RG7204 (PLX4032), a selective BRAFV600E inhibitor, displays potent antitumor activity in preclinical melanoma models. Cancer research 70, 5518-5527 (2010).
9. Hatzivassiliou, G., et al. RAF inhibitors prime wild-type RAF to activate the MAPK pathway and enhance growth. Nature 464, 431-435 (2010).
10. Heidorn, S. J., et al. Kinase-dead BRAF and oncogenic RAS cooperate to drive tumor progression through CRAF. Cell 140, 209-221 (2010).
11. Poulikakos, P. I., Zhang, C., Bollag, G., Shokat, K. M. & Rosen, N. RAF inhibitors transactivate RAF dimers and ERK signalling in cells with wild-type BRAF. Nature 464, 427-430 (2010).
12. Murphy, E. A., et al. Disruption of angiogenesis and tumor growth with an orally active drug that stabilizes the inactive state of PDGFRbeta/B-RAF. Proceedings of the National Academy of Sciences of the United States of America 107, 4299-4304 (2010).
13. Aaronson, S. A. Growth factors and cancer. Science (New York, N.Y. 254, 1146-1153 (1991).
14. Alavi, A., Hood, J. D., Frausto, R., Stupack, D. G. & Cheresh, D. A. Role of Raf in vascular protection from distinct apoptotic stimuli. Science (New York, N.Y. 301, 94-96 (2003).
15. Alavi, A. S., Acevedo, L., Min, W. & Cheresh, D. A. Chemoresistance of endothelial cells induced by basic fibroblast growth factor depends on Raf-1-mediated inhibition of the proapoptotic kinase, ASK1. Cancer research 67, 2766-2772 (2007).
16. McGlynn, L. M., et al. Ras/Raf-1/MAPK pathway mediates response to tamoxifen but not chemotherapy in breast cancer patients. Clin Cancer Res 15, 1487-1495 (2009).
17. Mikula, M., et al. Embryonic lethality and fetal liver apoptosis in mice lacking the c-raf-1 gene. The EMBO journal 20, 1952-1962 (2001).
18. Weber, C. K., Slupsky, J. R., Kalmes, H. A. & Rapp, U. R. Active Ras induces heterodimerization of cRaf and BRaf. Cancer research 61, 3595-3598 (2001).
19. Borysov, S. I., Cheng, A. W. & Guadagno, T. M. B-Raf is critical for MAPK activation during mitosis and is regulated in an M phase-dependent manner in Xenopus egg extracts. The Journal of biological chemistry 281, 22586-22596 (2006).
20. Borysov, S. I. & Guadagno, T. M. A novel role for Cdk1/cyclin B in regulating B-raf activation at mitosis. Molecular biology of the cell 19, 2907-2915 (2008).
21. Borysova, M. K., Cui, Y., Snyder, M. & Guadagno, T. M. Knockdown of B-Raf impairs spindle formation and the mitotic checkpoint in human somatic cells. Cell cycle (Georgetown, Tex. 7, 2894-2901 (2008).
22. Cui, Y., Borysova, M. K., Johnson, J. O. & Guadagno, T. M. Oncogenic B-Raf(V600E) induces spindle abnormalities, supernumerary centrosomes, and aneuploidy in human melanocytic cells. Cancer research 70, 675-684 (2010).
23. Cui, Y. & Guadagno, T. M. B-Raf(V600E) signaling deregulates the mitotic spindle checkpoint through stabilizing Mps1 levels in melanoma cells. Oncogene 27, 3122-3133 (2008).
24. Malumbres, M. & Barbacid, M. Cell cycle kinases in cancer. Current opinion in genetics & development 17, 60-65 (2007).
25. Nigg, E. A. Mitotic kinases as regulators of cell division and its checkpoints. Nature reviews 2, 21-32 (2001).
26. Taylor, S. & Peters, J. M. Polo and Aurora kinases: lessons derived from chemical biology. Current opinion in cell biology 20, 77-84 (2008).
27. Macurek, L., et al. Polo-like kinase-1 is activated by aurora A to promote checkpoint recovery. Nature 455, 119-123 (2008).
28. Petronczki, M., Lenart, P. & Peters, J. M. Polo on the Rise-from Mitotic Entry to Cytokinesis with Plk1. Developmental cell 14, 646-659 (2008).
29. Seki, A., Coppinger, J. A., Jang, C. Y., Yates, J. R. & Fang, G. Bora and the kinase Aurora a cooperatively activate the kinase Plk1 and control mitotic entry. Science (New York, N.Y. 320, 1655-1658 (2008).
30. Knauf, J. A., et al. Oncogenic RAS induces accelerated transition through G2/M and promotes defects in the G2 DNA damage and mitotic spindle checkpoints. The Journal of biological chemistry 281, 3800-3809 (2006).
31. Luo, J., et al. A genome-wide RNAi screen identifies multiple synthetic lethal interactions with the Ras oncogene. Cell 137, 835-848 (2009).
32. McInnes, C., et al Inhibitors of Polo-like kinase reveal roles in spindle-pole maintenance. Nature chemical biology 2, 608-617 (2006).
33. Santamaria, A., et al. Use of the novel Plk1 inhibitor ZK-thiazolidinone to elucidate functions of Plk1 in early and late stages of mitosis. Molecular biology of the cell 18, 4024-4036 (2007).
34. Steegmaier, M., et al. BI 2536, a potent and selective inhibitor of polo-like kinase 1, inhibits tumor growth in vivo. Curr Biol 17, 316-322 (2007).
35. Strebhardt, K. Multifaceted polo-like kinases: drug targets and antitargets for cancer therapy. Nat Rev Drug Discov 9, 643-660 (2010).
36. Arnaud, L., Pines, J. & Nigg, E. A. GFP tagging reveals human Polo-like kinase 1 at the kinetochore/centromere region of mitotic chromosomes. Chromosoma 107, 424-429 (1998).
37. Sumara, I., et al. Roles of polo-like kinase 1 in the assembly of functional mitotic spindles. Curr Biol 14, 1712-1722 (2004).
38. Huser, M., et al. MEK kinase activity is not necessary for Raf-1 function. The EMBO journal 20, 1940-1951 (2001).
39. Hindley, A. & Kolch, W. Extracellular signal regulated kinase (ERK)/mitogen activated protein kinase (MAPK)-independent functions of Raf kinases. Journal of cell science 115, 1575-1581 (2002).
40. Kamata, T., et al. BRAF Inactivation Drives Aneuploidy by Deregulating CRAF. Cancer research (2010).

A number of embodiments of the invention have been described. Nevertheless, it will be understood that various modifications may be made without departing from the spirit and scope of the invention. Accordingly, other embodiments are within the scope of the following claims.

What is claimed is:

1. A method for regulating or modulating cell growth or mitosis, angiogenesis, or regulating or modulating phospho-5338 CRAF localization to centrosomes/mitotic spindle poles, comprising
(1) (a) providing a composition comprising or consisting of:
(i) an allosteric regulator of a c-RAF kinase protein,
wherein the allosteric c-RAF regulator comprises an allosteric c-RAF inhibitor or an allosteric c-RAF activator, or
(ii) a composition or an agent which inhibits or prevents localization of a c-RAF to a mitotic spindle or mid-body; and
(b) administering a sufficient amount of the composition to the cell to regulate or modulate cell growth or mitosis,
wherein administering an allosteric c-RAF activator increases or unregulates cell growth or mitosis, or angiogenesis, or blocks G2M arrest,
and administering an allosteric c-RAF inhibitor, or a composition or an agent which inhibits or prevents localization of a c-RAF to a mitotic spindle, or inhibits, suppresses or decreases cell growth or mitosis, or inhibits or suppresses angiogenesis, or causes cell growth arrest,
and administering an allosteric c-RAF inhibitor inhibits phospho-5338 CRAF localization to centrosomes/mitotic spindle poles; or
(2) the method of (1), wherein the composition comprises a pharmaceutical composition administered in vivo; or
(3) the method of (1) or (2), wherein the composition is formulated for administration intravenously (IV), parenterally, nasally, topically, orally, or by liposome or vessel-targeted nanoparticle delivery;
(4) the method of any of (1) to (3), wherein the allosteric c-RAF inhibitor is not an ATP-competitive RAF inhibitor;
(5) the method of any of (1) to (3), wherein the allosteric c-RAF inhibitor or composition or agent which inhibits or prevents localization of a c-RAF to a mitotic spindle, acts by promoting G2M arrest, wherein optionally the G2M arrest is effected by the allosteric c-RAF inhibitor blocking association of c-RAF with a mitotic spindle and/or a mid-body during cell mitosis;
(6) the method of any of (1) to (5), wherein administering an allosteric c-RAF inhibitor, or a composition or an agent which inhibits or prevents localization of a c-RAF to a mitotic spindle, decreases, slows or blocks cancer cell growth;
(7) the method of any of (1) to (5), wherein administering an allosteric c-RAF inhibitor, or a composition or an agent which inhibits or prevents localization of a c-RAF to a mitotic spindle, decreases, slows or blocks new blood vessel growth, neovascularization or angiogenesis; or
(7) the method of any of (1) to (5), wherein administering an allosteric c-RAF inhibitor, or a composition or an agent which inhibits or prevents localization of a c-RAF to a mitotic spindle, treats or ameliorates conditions that are responsive to blocking or slowing cell growth, and/or the development of neovascularization or new blood vessels,
wherein the method optionally reduces, treats or ameliorates the level of disease in a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

2. A method for reducing, treating or ameliorating a condition or disease responsive to slowing, decreasing the rate of, arresting or inhibiting cell growth, comprising:
(1) (a) providing a composition comprising or consisting of:
(i) an allosteric regulator of a c-RAF kinase protein,
wherein the allosteric RAF regulator comprises an allosteric c-RAF inhibitor or an allosteric c-RAF activator, or
(ii) a composition or an agent which inhibits or prevents localization of a c-RAF to a mitotic spindle; and
(b) administering a sufficient amount of the composition to the cell to regulate or modulate cell growth or mitosis,
wherein administering an allosteric c-RAF inhibitor, or a composition or an agent which inhibits or prevents localization of a c-RAF to a mitotic spindle, inhibits, suppresses or decreases cell growth or mitosis, or causes cell growth arrest,
wherein administering an allosteric c-RAF activator increases or unregulates cell growth or mitosis, or blocks G2M arrest; or
(2) the method of (1), wherein the composition comprises a pharmaceutical composition administered in vivo; or
(3) the method of (1) or (2), wherein the composition is formulated for administration intravenously (IV), parenterally, orally, or by liposome or vessel-targeted nanoparticle delivery.
(4) the method of any of (1) to (3), wherein the allosteric c-RAF inhibitor is not an ATP-competitive RAF inhibitor;
(5) the method of any of (1) to (3), wherein the allosteric c-RAF inhibitor or composition or agent which inhibits or prevents localization of a c-RAF to a mitotic spindle, acts by promoting G2M arrest, wherein optionally the G2M arrest is effected by the allosteric c-RAF inhibitor blocking association of c-RAF with a mitotic spindle and/or a mid-body during cell mitosis;
(6) the method of any of (1) to (5), wherein administering an allosteric c-RAF inhibitor, or a composition or an agent which inhibits or prevents localization of a c-RAF to a mitotic spindle, decreases, slows or blocks cancer cell growth;
(7) the method of any of (1) to (6), wherein administering an allosteric c-RAF inhibitor, or a composition or an agent which inhibits or prevents localization of a c-RAF to a mitotic spindle, decreases, slows or blocks new blood vessel growth, neovascularization or angiogenesis; or
(8) the method of any of (1) to (7), wherein administering an allosteric c-RAF inhibitor, or a composition or an agent which inhibits or prevents localization of a c-RAF to a mitotic spindle, treats or ameliorates conditions that are responsive to blocking or slowing cell growth, and/or the development of neovascularization or new blood vessels,
wherein the method optionally reduces, treats or ameliorates the level of disease in a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

3. A kit comprising a composition comprising or consisting of: (i) an allosteric regulator of a c-RAF kinase protein, wherein the allosteric c-RAF regulator comprises an allosteric c-RAF inhibitor or an allosteric c-RAF activator, or (ii) a composition or an agent which inhibits or prevents localization of a c-RAF to a mitotic spindle; and optionally further comprising instructions for practicing the method of claim 1.

4. A kit comprising a composition comprising or consisting of: (i) an allosteric regulator of a c-RAF kinase protein, wherein the allosteric c-RAF regulator comprises an allosteric c-RAF inhibitor or an allosteric c-RAF activator, or (ii) a composition or an agent which inhibits or prevents localization of a c-RAF to a mitotic spindle; and optionally further comprising instructions for practicing the method of claim 2.

5. A method for determining whether an individual or a patient would benefit from administration of an allosteric c-RAF inhibitor, or determining the therapeutic efficacy of an agent that blocks cell proliferation at the mitotic phase, comprising:
(a) detecting a serine-338 phosphorylated c-RAF, or detecting a serine-338 phosphorylated c-RAF localized to the mitotic spindle, wherein optionally the detection is by analysis or visualization of a biopsy or other tissue sample or a pathology slide taken from the patient or individual,
wherein detection of a serine-338 phosphorylated c-RAF, or detection of a serine-338 phosphorylated c-RAF localized to the mitotic spindle, indicates: that the individual or patient will be responsive to the allosteric c-RAF inhibitor, or that the agent that blocks cell proliferation at the mitotic phase will have a therapeutic efficacy; or
(b) the method of (a), wherein an antibody that can specifically bind to a serine-338 phosphorylated c-RAF is used to detect a serine-338 phosphorylated c-RAF localized to the mitotic spindle, or the serine-338 phosphorylated c-RAF.

6. A method for blocking proliferating cells in mitosis by preventing c-RAF localization to a mitotic spindle or a mid-body of a mammalian cell comprising:
(a) providing a composition comprising or consisting of:
(i) an allosteric c-RAF inhibitor of a c-RAF kinase protein, or
(ii) a composition or an agent which inhibits or prevents localization of a c-RAF to a mitotic spindle or a mid-body; and
(b) administering a sufficient amount of the composition to the mammalian cell to block proliferating mammalian cells in mitosis by preventing c-RAF localization to the mitotic spindle or mid-body.

7. A method for inhibiting or impairing a polo-like kinase 1 (Plk1 kinase) activity and/or inhibiting or impairing Plk1 kinase accumulation at a kinetochore in a mammalian cell comprising:
(a) providing a composition comprising or consisting of:
(i) an allosteric c-RAF inhibitor of a c-RAF kinase protein, or
(ii) a composition or an agent which inhibits or prevents localization of a c-RAF to a mitotic spindle or a mid-body; and
(b) administering a sufficient amount of the composition to the mammalian cell to inhibit or impair the Plk1 kinase activity, or inhibit or impair Plk1 kinase accumulation at the kinetochore, in the mammalian cell.

8. The method of claim 1, wherein the allosteric RAF inhibitor comprises or consists of, or is selected from the group consisting of, any one of the following compounds, or equivalents thereof:
(a) N-(3-(4-(pyridin-3-yl)pyrimidin-2-ylamino)-4-methylphenyl)-4-((4-methylpiperazin-1-yl)methyl)benzamide;
(b) 1-(4-(2-(methylcarbamoyl)pyridin-4-yloxy)phenyl)-3-(4-chloro-3-(trifluoromethyl)phenyl)urea;
(c) 6-(4-(5-(3-(trifluoromethyl)phenylamino)-4H-1,2,4-triazol-3-yl)phenoxy)pyrimidin-4-amine;
(d) 6-(4-(5-(3-(trifluoromethyl)phenylamino)-4H-1,2,4-triazol-3-yl)phenoxy)pyrimidine-2,4-diamine;
(e) 6-(4-(5-(4-chloro-3-(trifluoromethyl)phenylamino)-4H-1,2,4-triazol-3-yl)phenoxy)pyrimidine-2,4-diamine;
(f) 6-(4-(5-(3-(trifluoromethyl)phenylamino)-4H-1,2,4-triazol-3-yl)phenoxy)-2-(methylthio)pyrimidin-4-amine;
(g) 5-(4-(2-(methylthio)pyrimidin-4-yloxy)phenyl)-N-(3-(trifluoromethyl)phenyl)-4H-1,2,4-triazol-3-amine;
(h) 6-(4-(5-(4-chloro-3-(trifluoromethyl)phenylamino)-4H-1,2,4-triazol-3-yl)phenoxy)-2-(methylthio)pyrimidin-4-amine;
(i) 4-(4-(5-(3-(trifluoromethyl)phenylamino)-4H-1,2,4-triazol-3-yl)phenoxy)-6-methoxypyrimidin-2-amine;
(j) 6-(4-(5-(3-(trifluoromethyl)phenylamino)-1,3,4-oxadiazol-2-yl)phenoxy)pyrimidine-2,4-Diamine;
(k) 6-(4-(5-(3-(trifluoromethyl)phenylamino)-1,3,4-oxadiazol-2-yl)phenoxy)-2-(methylthio)pyrimidin-4-amine;
(l) (Z)-3-((3,5-dimethyl-1 Hpyrrol-2-yl)methylene)indolin-2-one;
(m) 3-(3,5-dibromo-4-hydroxybenzylidene)-5-iodoindolin-2-one;
(n) 2-(methylsulfanyl)-6-[4-(2-{[3-(trifluoromethyl)phenyl]amino}-1H-imidazol-5-yl)phenoxy]pyrimidin-4-amine (also called “KG5”), or 2-(methylsulfanyl)-6-[4-(2-{[3-(trifluoromethyl)phenyl]amino}-1H-imidazol-5-yl)phenoxy]pyrimidin-4-amine (also called “K38-B, or KG38”), having the structure: 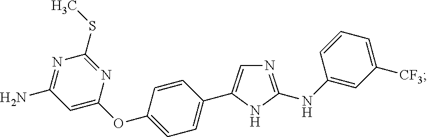 [SMILES: CSc1nc(N)cc(Oc2ccc(-c3cnc(Nc4cccc(C(F)(F)F)c4)[nH]3)cc2)n1]
(o) 6-(4-{5-[(2,3-dimethylphenyl)amino]-4H-1,2,4-triazol-3-yl}phenoxy)-2-(methylsulfanyl)pyrimidin-4-amine (also called “H3-11”), having the structure: 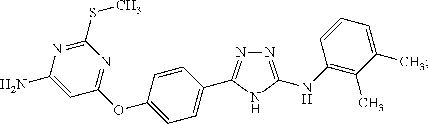 [SMILES: CSc1nc(N)cc(Oc2ccc(-c3nnc(Nc4cccc(C)c4C)[nH]3)cc2)n1]
(p) 2-(methylsulfanyl)-6-{4-[5-(5,6,7,8-tetrahydronaphthalen-1-ylamino)-4H-1,2,4-triazol-3-yl]phenoxy}pyrimidin-4-amine (also called “H3-21”), having the structure: 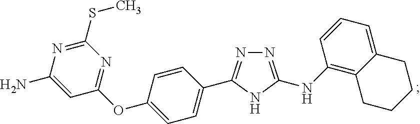 [SMILES: CSc1nc(N)cc(Oc2ccc(-c3nnc(Nc4cccc5c4CCCC5)[nH]3)cc2)n1]
(q) 6-(4-{5-[(3,4-dimethylphenyl)amino]-4H-1,2,4-triazol-3-yl}phenoxy)-2-(methylsulfanyl)pyrimidin-4-amine (also called “H3-9”), having the structure: 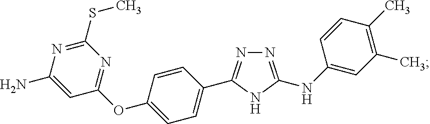 [SMILES: CSc1nc(N)cc(Oc2ccc(-c3nnc(Nc4ccc(C)c(C)c4)[nH]3)cc2)n1]
(s) 6-(4-{5-[(3,5-dimethylphenyl)amino]-4H-1,2,4-triazol-3-yl}phenoxy)-2-(methylsulfanyl)pyrimidin-4-amine (also called “H3-7”), having the structure: 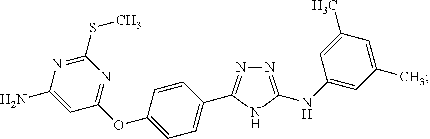 [SMILES: CSc1nc(N)cc(Oc2ccc(-c3nnc(Nc4cc(C)cc(C)c4)[nH]3)cc2)n1]
(s) 6-(4-{5-[(2,5-dimethylphenyl)amino]-4H-1,2,4-triazol-3-yl}phenoxy)-2-(methylsulfanyl)pyrimidin-4-amine (also called “H3-3”), having the structure: 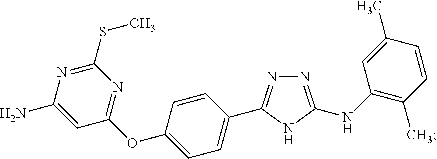 [SMILES: CSc1nc(N)cc(Oc2ccc(-c3nnc(Nc4cc(C)ccc4C)[nH]3)cc2)n1]
(t) 2-ethoxy-6-[4-(5-{[3-(trifluoromethyl)phenyl]amino}-4H-1,2,4-triazol-3-yl)phenoxy]pyrimidin-4-amine (also called “K1-72”), having the structure: 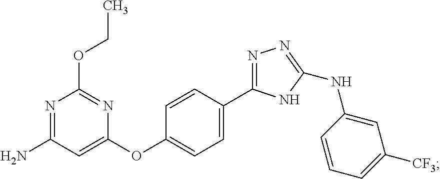 [SMILES: CCOc1nc(N)cc(Oc2ccc(-c3nnc(Nc4cccc(C(F)(F)F)c4)[nH]3)cc2)n1]
(u) a compound having a structural Formula I: 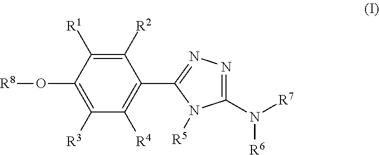
or a pharmaceutically acceptable salt or solvate thereof,
wherein:
R¹-R⁶ are independently selected from the group consisting of hydrogen, deuterium, alkyls, alkenyls, alkynyls, aryls, hetero-alkyls, hetero-alkenyls, hetero-alkynyls, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, thiols, sulfhydryls, sulfides, disulfides, sulfinyls, sulfos, thiocyanates, isothiocyanates, carbonothioyls, phosphinos, phosphonos, phosphates, silyls, and Si(OH)₃; and
R⁷-R⁸ are independently selected from the group consisting of cycloalkyl, cycloalkenyl, cycloalkynyl, aryl, heterocyle, and extended ring system;
(v) a genus of compounds as defined in step (u), with the proviso that the compound or specie is not a 6-(4-(5-(3-(trifluoromethyl)phenylamino)-4H-1,2,4-triazol-3-yl)phenoxy)-2-(methylthio)pyrimidin-4-amine (also can be designated “KG5”);
(w) a compound having structural Formula II: 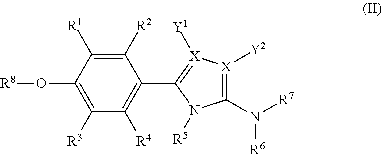
or a pharmaceutically acceptable salt or solvate thereof, wherein:
X is independently N or C, and wherein when X is N then Y is absent;
R¹-R⁶, Y¹ and Y², are independently selected from the group consisting of hydrogen, deuterium, alkyls, alkenyls, alkynyls, aryls, hetero-alkyls, hetero-alkenyls, hetero-alkynyls, alkoxys, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, thiols, thioethers, thioethers, sulfhydryls, sulfides, disulfides, sulfinyls, sulfos, thiocyanates, isothiocyanates, carbonothioyls, phosphinos, phosphonos, phosphates, silyls, and Si(OH)₃; and
R⁷-R⁸ are independently selected from the group consisting of cycloalkyl, cycloalkenyl, cycloalkynyl, aryl, heterocyle, and extended ring system;
(x) a genus of compounds having a structural Formula II, wherein:
R⁷ is an aryl having the structure: 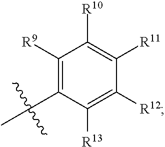
R⁸ is a heterocycle having the structure: 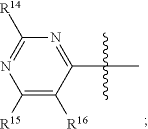
and
R⁹-R¹⁶ are independently selected from the group consisting of hydrogen, deuterium, alkyls, alkenyls, alkynyls, hetero-alkyls, hetero-alkenyls, hetero-alkynyls, alkoxys, halos, hydroxyls, carboxyls, carboxylates, aldehydes, haloformyls, esters, ethers, orthoesters, amines, azides, cyanates, isocyanates, nitrates, nitriles, isonitriles, nitrosos, nitros, nitrosooxy, pyridyls, sulfhydryls, sulfides, disulfides, sulfinyls, thiols, thioethers, sulfos, thiocyanates, isothiocyanates, carbonothioyls, phosphinos, phosphonos, phosphates, silyls, and Si(OH)₃;
(y) a genus of compounds having a structural Formula II, wherein:
R¹-R⁶, and R¹⁶ are independently selected from the group consisting of hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, amines, azides, cyanates, nitrates, nitriles, nitros, thiols, and phosphates;
R⁹-R¹³ are independently selected from the group consisting of hydrogen, deuterium, methyl and CF₃;
R¹⁴ is selected from the group consisting of (C₁-C₆)alkyls, (C₂-C₆)alkenyls, (C₂-C₆)alkynyls, hetero-(C₁-C₅)alkyls, hetero-(C₁-C₅)alkenyls, hetero-(C₁-C₅)alkynyls, alkoxys, ethers, carboxylic acid, amines, aldehyde, carbonyls, thiols, thioethers, esters, and azides, and
R¹⁵ is an amine; and/or
(z) the genus of compounds as defined in step (w) (based on structural Formula II) with the proviso that genus does not have (include) the compound or specie 6-(4-(5-(3-(trifluoromethyl)phenylamino)-4H-1,2,4-triazol-3-yl)phenoxy)-2-(methylthio)pyrimidin-4-amine (also can be designated “KG5”).

9. The method of claim 8, wherein:
R⁷ is an aryl having the structure: 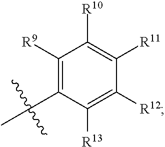
R⁸ is a heterocycle having the structure: 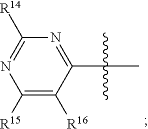
and/or
R⁹-R¹⁶ are independently selected from the group consisting of hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, orthoesters, amines, azides, cyanates, isocyanates, nitrates, nitriles, isonitriles, nitrosos, nitros, nitrosooxy, pyridyls, sulfhydryls, sulfides, disulfides, sulfinyls, thiols, sulfos, thiocyanates, isothiocyanates, carbonothioyls, phosphinos, phosphonos, phosphates, silyls, and Si(OH)₃.

10. The method of claim 9, wherein:
R¹-R⁶, R⁹-R¹¹, R¹³, and R¹⁶ are independently either hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, amines, azides, cyanates, nitrates, nitriles, nitros, thiols, and phosphates;
R¹² is CF₃;
R¹⁴ is SCH₃; and/or
R¹⁵ is NH₂.

11. The method of claim 2, wherein the allosteric RAF inhibitor comprises or consists of, or is selected from the group consisting of, any one of the following compounds, or equivalents thereof:
(a) N-(3-(4-(pyridin-3-yl)pyrimidin-2-ylamino)-4-methylphenyl)-4-((4-methylpiperazin-1-yl)methyl)benzamide;
(b) 1-(4-(2-(methylcarbamoyl)pyridin-4-yloxy)phenyl)-3-(4-chloro-3-(trifluoromethyl)phenyl)urea;
(c) 6-(4-(5-(3-(trifluoromethyl)phenylamino)-4H-1,2,4-triazol-3-yl)phenoxy)pyrimidin-4-amine;
(d) 6-(4-(5-(3-(trifluoromethyl)phenylamino)-4H-1,2,4-triazol-3-yl)phenoxy)pyrimidine-2,4-diamine;
(e) 6-(4-(5-(4-chloro-3-(trifluoromethyl)phenylamino)-4H-1,2,4-triazol-3-yl)phenoxy)pyrimidine-2,4-diamine;
(f) 6-(4-(5-(3-(trifluoromethyl)phenylamino)-4H-1,2,4-triazol-3-yl)phenoxy)-2-(methylthio)pyrimidin-4-amine;
(g) 5-(4-(2-(methylthio)pyrimidin-4-yloxy)phenyl)-N-(3-(trifluoromethyl)phenyl)-4H-1,2,4-triazol-3-amine;
(h) 6-(4-(5-(4-chloro-3-(trifluoromethyl)phenylamino)-4H-1,2,4-triazol-3-yl)phenoxy)-2-(methylthio)pyrimidin-4-amine;
(i) 4-(4-(5-(3-(trifluoromethyl)phenylamino)-4H-1,2,4-triazol-3-yl)phenoxy)-6-methoxypyrimidin-2-amine;
(j) 6-(4-(5-(3-(trifluoromethyl)phenylamino)-1,3,4-oxadiazol-2-yl)phenoxy)pyrimidine-2,4-Diamine;
(k) 6-(4-(5-(3-(trifluoromethyl)phenylamino)-1,3,4-oxadiazol-2-yl)phenoxy)-2-(methylthio)pyrimidin-4-amine;
(l) (Z)-3-((3,5-dimethyl-1 Hpyrrol-2-yl)methylene)indolin-2-one;
(m) 3-(3,5-dibromo-4-hydroxybenzylidene)-5-iodoindolin-2-one;
(n) 2-(methylsulfanyl)-6-[4-(2-{[3-(trifluoromethyl)phenyl]amino}-1H-imidazol-5-yl)phenoxy]pyrimidin-4-amine (also called “KG5”), or 2-(methylsulfanyl)-6-[4-(2-{[3-(trifluoromethyl)phenyl]amino}-1H-imidazol-5-yl)phenoxy]pyrimidin-4-amine (also called “K38-B, or KG38”), having the structure: 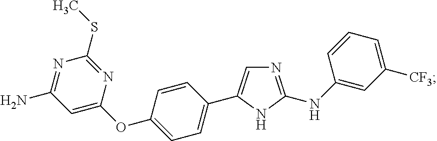 [SMILES: CSc1nc(N)cc(Oc2ccc(-c3cnc(Nc4cccc(C(F)(F)F)c4)[nH]3)cc2)n1]
(o) 6-(4-{5-[(2,3-dimethylphenyl)amino]-4H-1,2,4-triazol-3-yl}phenoxy)-2-(methylsulfanyl)pyrimidin-4-amine (also called “H3-11”), having the structure: 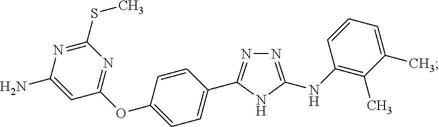 [SMILES: CSc1nc(N)cc(Oc2ccc(-c3nnc(Nc4cccc(C)c4C)[nH]3)cc2)n1]
(p) 2-(methylsulfanyl)-6-{4-[5-(5,6,7,8-tetrahydronaphthalen-1-ylamino)-4H-1,2,4-triazol-3-yl]phenoxy}pyrimidin-4-amine (also called “H3-21”), having the structure: 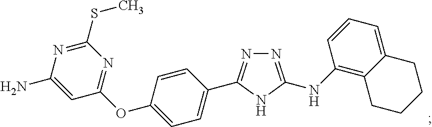 [SMILES: CSc1nc(N)cc(Oc2ccc(-c3nnc(Nc4cccc5c4CCCC5)[nH]3)cc2)n1]
(q) 6-(4-{5-[(3,4-dimethylphenyl)amino]-4H-1,2,4-triazol-3-yl}phenoxy)-2-(methylsulfanyl)pyrimidin-4-amine (also called “H3-9”), having the structure: 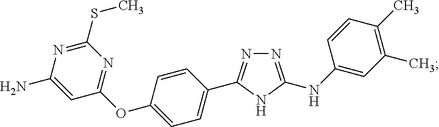 [SMILES: CSc1nc(N)cc(Oc2ccc(-c3nnc(Nc4ccc(C)c(C)c4)[nH]3)cc2)n1]
(t) 6-(4-{5-[(3,5-dimethylphenyl)amino]-4H-1,2,4-triazol-3-yl}phenoxy)-2-(methylsulfanyl)pyrimidin-4-amine (also called “H3-7”), having the structure: 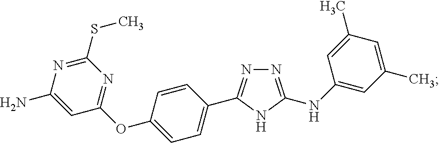 [SMILES: CSc1nc(N)cc(Oc2ccc(-c3nnc(Nc4cc(C)cc(C)c4)[nH]3)cc2)n1]
(s) 6-(4-{5-[(2,5-dimethylphenyl)amino]-4H-1,2,4-triazol-3-yl}phenoxy)-2-(methylsulfanyl)pyrimidin-4-amine (also called “H3-3”), having the structure: 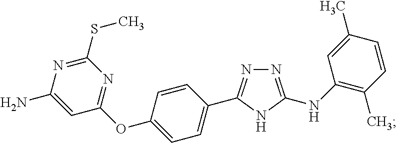 [SMILES: CSc1nc(N)cc(Oc2ccc(-c3nnc(Nc4cc(C)ccc4C)[nH]3)cc2)n1]
(t) 2-ethoxy-6-[4-(5-{[3-(trifluoromethyl)phenyl]amino}-4H-1,2,4-triazol-3-yl)phenoxy]pyrimidin-4-amine (also called “K1-72”), having the structure: 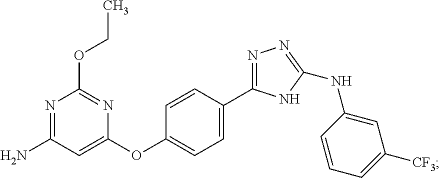 [SMILES: CCOc1nc(N)cc(Oc2ccc(-c3nnc(Nc4cccc(C(F)(F)F)c4)[nH]3)cc2)n1]
(u) a compound having a structural Formula I: 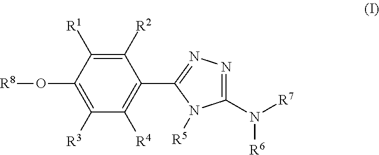
or a pharmaceutically acceptable salt or solvate thereof,
wherein:
R¹-R⁶ are independently selected from the group consisting of hydrogen, deuterium, alkyls, alkenyls, alkynyls, aryls, hetero-alkyls, hetero-alkenyls, hetero-alkynyls, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, thiols, sulfhydryls, sulfides, disulfides, sulfinyls, sulfos, thiocyanates, isothiocyanates, carbonothioyls, phosphinos, phosphonos, phosphates, silyls, and Si(OH)₃; and
R⁷-R⁸ are independently selected from the group consisting of cycloalkyl, cycloalkenyl, cycloalkynyl, aryl, heterocyle, and extended ring system;
(v) a genus of compounds as defined in step (u), with the proviso that the compound or specie is not a 6-(4-(5-(3-(trifluoromethyl)phenylamino)-4H-1,2,4-triazol-3-yl)phenoxy)-2-(methylthio)pyrimidin-4-amine (also can be designated “KG5”);
(w) a compound having structural Formula II: 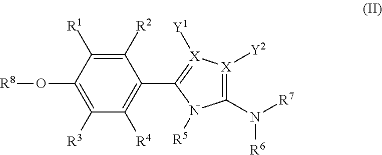
or a pharmaceutically acceptable salt or solvate thereof,
wherein:
X is independently N or C, and wherein when X is N then Y is absent;
R¹-R⁶, Y¹ and Y², are independently selected from the group consisting of hydrogen, deuterium, alkyls, alkenyls, alkynyls, aryls, hetero-alkyls, hetero-alkenyls, hetero-alkynyls, alkoxys, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, thiols, thioethers, thioethers, sulfhydryls, sulfides, disulfides, sulfinyls, sulfos, thiocyanates, isothiocyanates, carbonothioyls, phosphinos, phosphonos, phosphates, silyls, and Si(OH)₃; and
R⁷-R⁸ are independently selected from the group consisting of cycloalkyl, cycloalkenyl, cycloalkynyl, aryl, heterocyle, and extended ring system;
(x) a genus of compounds having a structural Formula II, wherein:
R⁷ is an aryl having the structure: 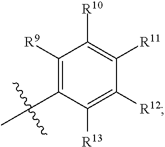
R⁸ is a heterocycle having the structure: 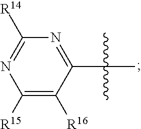
and
R⁹-R¹⁶ are independently selected from the group consisting of hydrogen, deuterium, alkyls, alkenyls, alkynyls, hetero-alkyls, hetero-alkenyls, hetero-alkynyls, alkoxys, halos, hydroxyls, carboxyls, carboxylates, aldehydes, haloformyls, esters, ethers, orthoesters, amines, azides, cyanates, isocyanates, nitrates, nitriles, isonitriles, nitrosos, nitros, nitrosooxy, pyridyls, sulfhydryls, sulfides, disulfides, sulfinyls, thiols, thioethers, sulfos, thiocyanates, isothiocyanates, carbonothioyls, phosphinos, phosphonos, phosphates, silyls, and Si(OH)₃;
(y) a genus of compounds having a structural Formula II, wherein:
R¹-R⁶, and R¹⁶ are independently selected from the group consisting of hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, amines, azides, cyanates, nitrates, nitriles, nitros, thiols, and phosphates;
R⁹-R¹³ are independently selected from the group consisting of hydrogen, deuterium, methyl and CF₃;
R¹⁴ is selected from the group consisting of (C₁-C₆)alkyls, (C₂-C₆)alkenyls, (C₂-C₆)alkynyls, hetero-(C₁-C₅)alkyls, hetero-(C₁-C₅)alkenyls, hetero-(C₁-C₅)alkynyls, alkoxys, ethers, carboxylic acid, amines, aldehyde, carbonyls, thiols, thioethers, esters, and azides, and
R¹⁵ is an amine; and/or
(z) the genus of compounds as defined in step (w) (based on structural Formula II) with the proviso that genus does not have (include) the compound or specie 6-(4-(5-(3-(trifluoromethyl)phenylamino)-4H-1,2,4-triazol-3-yl)phenoxy)-2-(methylthio)pyrimidin-4-amine (also can be designated “KG5”).

12. The method of claim 11, wherein:
R⁷ is an aryl having the structure: 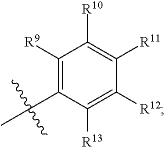
R⁸ is a heterocycle having the structure: 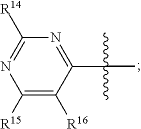
and/or
R⁹-R¹⁶ are independently selected from the group consisting of hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, orthoesters, amines, azides, cyanates, isocyanates, nitrates, nitriles, isonitriles, nitrosos, nitros, nitrosooxy, pyridyls, sulfhydryls, sulfides, disulfides, sulfinyls, thiols, sulfos, thiocyanates, isothiocyanates, carbonothioyls, phosphinos, phosphonos, phosphates, silyls, and Si(OH)₃.

13. The method of claim 12, wherein:
R¹-R⁶, R⁹-R¹¹, R¹³, and R¹⁶ are independently either hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, amines, azides, cyanates, nitrates, nitriles, nitros, thiols, and phosphates;
R¹² is CF₃;
R¹⁴ is SCH₃; and/or
R¹⁵ is NH₂.

14. The method of claim 6, wherein the allosteric RAF inhibitor comprises or consists of, or is selected from the group consisting of, any one of the following compounds, or equivalents thereof:
(a) N-(3-(4-(pyridin-3-yl)pyrimidin-2-ylamino)-4-methylphenyl)-4-((4-methylpiperazin-1-yl)methyl)benzamide;
(b) 1-(4-(2-(methylcarbamoyl)pyridin-4-yloxy)phenyl)-3-(4-chloro-3-(trifluoromethyl)phenyl)urea;
(c) 6-(4-(5-(3-(trifluoromethyl)phenylamino)-4H-1,2,4-triazol-3-yl)phenoxy)pyrimidin-4-amine;
(d) 6-(4-(5-(3-(trifluoromethyl)phenylamino)-4H-1,2,4-triazol-3-yl)phenoxy)pyrimidine-2,4-diamine;
(e) 6-(4-(5-(4-chloro-3-(trifluoromethyl)phenylamino)-4H-1,2,4-triazol-3-yl)phenoxy)pyrimidine-2,4-diamine;
(f) 6-(4-(5-(3-(trifluoromethyl)phenylamino)-4H-1,2,4-triazol-3-yl)phenoxy)-2-(methylthio)pyrimidin-4-amine;
(g) 5-(4-(2-(methylthio)pyrimidin-4-yloxy)phenyl)-N-(3-(trifluoromethyl)phenyl)-4H-1,2,4-triazol-3-amine;
(h) 6-(4-(5-(4-chloro-3-(trifluoromethyl)phenylamino)-4H-1,2,4-triazol-3-yl)phenoxy)-2-(methylthio)pyrimidin-4-amine;
(i) 4-(4-(5-(3-(trifluoromethyl)phenylamino)-4H-1,2,4-triazol-3-yl)phenoxy)-6-methoxypyrimidin-2-amine;
(j) 6-(4-(5-(3-(trifluoromethyl)phenylamino)-1,3,4-oxadiazol-2-yl)phenoxy)pyrimidine-2,4-Diamine;
(k) 6-(4-(5-(3-(trifluoromethyl)phenylamino)-1,3,4-oxadiazol-2-yl)phenoxy)-2-(methylthio)pyrimidin-4-amine;
(l) (Z)-3-((3,5-dimethyl-1 Hpyrrol-2-yl)methylene)indolin-2-one;
(m) 3-(3,5-dibromo-4-hydroxybenzylidene)-5-iodoindolin-2-one;
(n) 2-(methylsulfanyl)-6-[4-(2-{[3-(trifluoromethyl)phenyl]amino}-1H-imidazol-5-yl)phenoxy]pyrimidin-4-amine (also called “KG5”), or 2-(methylsulfanyl)-6-[4-(2-{[3-(trifluoromethyl)phenyl]amino}-1H-imidazol-5-yl)phenoxy]pyrimidin-4-amine (also called “K38-B, or KG38”), having the structure: 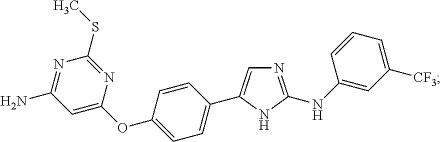 [SMILES: CSc1nc(N)cc(Oc2ccc(-c3cnc(Nc4cccc(C(F)(F)F)c4)[nH]3)cc2)n1]
(o) 6-(4-{5-[(2,3-dimethylphenyl)amino]-4H-1,2,4-triazol-3-yl}phenoxy)-2-(methylsulfanyl)pyrimidin-4-amine (also called “H3-11”), having the structure: 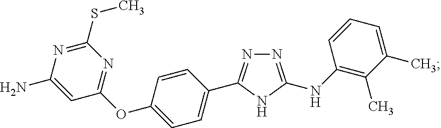 [SMILES: CSc1nc(N)cc(Oc2ccc(-c3nnc(Nc4cccc(C)c4C)[nH]3)cc2)n1]
(p) 2-(methylsulfanyl)-6-{4-[5-(5,6,7,8-tetrahydronaphthalen-1-ylamino)-4H-1,2,4-triazo-3-yl]phenoxy}pyrimidin-4-amine (also called “H3-21”), having the structure: 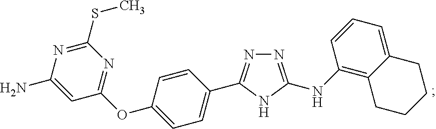 [SMILES: CSc1nc(N)cc(Oc2ccc(-c3nnc(Nc4cccc5c4CCCC5)[nH]3)cc2)n1]
(q) 6-(4-{5-[(3,4-dimethylphenyl)amino]-4H-1,2,4-triazol-3-yl}phenoxy)-2-(methylsulfanyl)pyrimidin-4-amine (also called “H3-9”), having the structure: 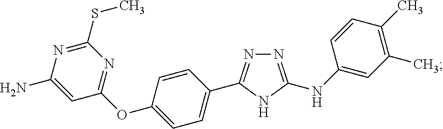 [SMILES: CSc1nc(N)cc(Oc2ccc(-c3nnc(Nc4ccc(C)c(C)c4)[nH]3)cc2)n1]
(u) 6-(4-{5-[(3,5-dimethylphenyl)amino]-4H-1,2,4-triazol-3-yl}phenoxy)-2-(methylsulfanyl)pyrimidin-4-amine (also called “H3-7”), having the structure: 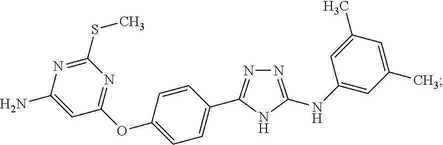 [SMILES: CSc1nc(N)cc(Oc2ccc(-c3nnc(Nc4cc(C)cc(C)c4)[nH]3)cc2)n1]
(s) 6-(4-{5-[(2,5-dimethylphenyl)amino]-4H-1,2,4-triazol-3-yl}phenoxy)-2-(methylsulfanyl)pyrimidin-4-amine (also called “H3-3”), having the structure: 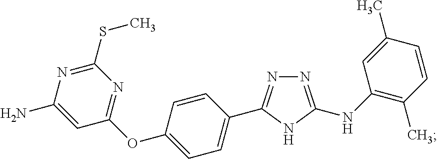 [SMILES: CSc1nc(N)cc(Oc2ccc(-c3nnc(Nc4cc(C)ccc4C)[nH]3)cc2)n1]
(t) 2-ethoxy-6-[4-(5-{[3-(trifluoromethyl)phenyl]amino}-4H-1,2,4-triazol-3-yl)phenoxy]pyrimidin-4-amine (also called “K1-72”), having the structure: 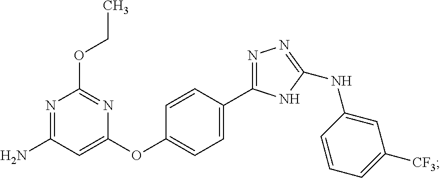 [SMILES: CCOc1nc(N)cc(Oc2ccc(-c3nnc(Nc4cccc(C(F)(F)F)c4)[nH]3)cc2)n1]
(u) a compound having a structural Formula I: 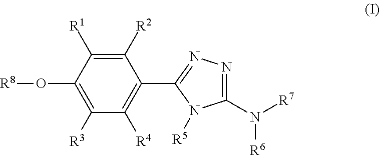
or a pharmaceutically acceptable salt or solvate thereof,
wherein:
R¹-R⁶ are independently selected from the group consisting of hydrogen, deuterium, alkyls, alkenyls, alkynyls, aryls, hetero-alkyls, hetero-alkenyls, hetero-alkynyls, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, thiols, sulfhydryls, sulfides, disulfides, sulfinyls, sulfos, thiocyanates, isothiocyanates, carbonothioyls, phosphinos, phosphonos, phosphates, silyls, and Si(OH)₃; and
R²-R⁸ are independently selected from the group consisting of cycloalkyl, cycloalkenyl, cycloalkynyl, aryl, heterocyle, and extended ring system;
(v) a genus of compounds as defined in step (u), with the proviso that the compound or specie is not a 6-(4-(5-(3-(trifluoromethyl)phenylamino)-4H-1,2,4-triazol-3-yl)phenoxy)-2-(methylthio)pyrimidin-4-amine (also can be designated “KG5”);
(w) a compound having structural Formula II: 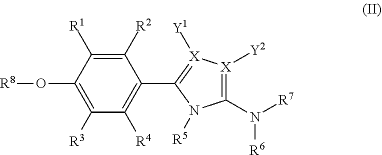
or a pharmaceutically acceptable salt or solvate thereof,
wherein:
X is independently N or C, and wherein when X is N then Y is absent;
R¹-R⁶, Y¹ and Y², are independently selected from the group consisting of hydrogen, deuterium, alkyls, alkenyls, alkynyls, aryls, hetero-alkyls, hetero-alkenyls, hetero-alkynyls, alkoxys, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, thiols, thioethers, thioethers, sulfhydryls, sulfides, disulfides, sulfinyls, sulfos, thiocyanates, isothiocyanates, carbonothioyls, phosphinos, phosphonos, phosphates, silyls, and Si(OH)₃; and
R⁷-R⁸ are independently selected from the group consisting of cycloalkyl, cycloalkenyl, cycloalkynyl, aryl, heterocyle, and extended ring system;
(x) a genus of compounds having a structural Formula II, wherein:
R⁷ is an aryl having the structure: 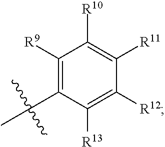
R⁸ is a heterocycle having the structure: 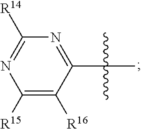
and
R⁹-R¹⁶ are independently selected from the group consisting of hydrogen, deuterium, alkyls, alkenyls, alkynyls, hetero-alkyls, hetero-alkenyls, hetero-alkynyls, alkoxys, halos, hydroxyls, carboxyls, carboxylates, aldehydes, haloformyls, esters, ethers, orthoesters, amines, azides, cyanates, isocyanates, nitrates, nitriles, isonitriles, nitrosos, nitros, nitrosooxy, pyridyls, sulfhydryls, sulfides, disulfides, sulfinyls, thiols, thioethers, sulfos, thiocyanates, isothiocyanates, carbonothioyls, phosphinos, phosphonos, phosphates, silyls, and Si(OH)₃;
(y) a genus of compounds having a structural Formula II, wherein:
R¹-R⁶, and R¹⁶ are independently selected from the group consisting of hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, amines, azides, cyanates, nitrates, nitriles, nitros, thiols, and phosphates;
R⁹-R¹³ are independently selected from the group consisting of hydrogen, deuterium, methyl and CF₃;
R¹⁴ is selected from the group consisting of (C₁-C₆)alkyls, (C₂-C₆)alkenyls, (C₂-C₆)alkynyls, hetero-(C₁-C₅)alkyls, hetero-(C₁-C₅)alkenyls, hetero-(C₁-C₅)alkynyls, alkoxys, ethers, carboxylic acid, amines, aldehyde, carbonyls, thiols, thioethers, esters, and azides, and
R¹⁵ is an amine; and/or
(z) the genus of compounds as defined in step (w) (based on structural Formula II) with the proviso that genus does not have (include) the compound or specie 6-(4-(5-(3-(trifluoromethyl)phenylamino)-4H-1,2,4-triazol-3-yl)phenoxy)-2-(methylthio)pyrimidin-4-amine (also can be designated “KG5”).

15. The method of claim 14, wherein:
R⁷ is an aryl having the structure: 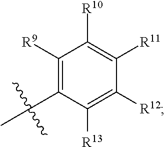
R⁸ is a heterocycle having the structure: 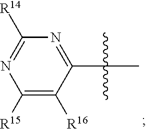
and/or
R⁹-R¹⁶ are independently selected from the group consisting of hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, orthoesters, amines, azides, cyanates, isocyanates, nitrates, nitriles, isonitriles, nitrosos, nitros, nitrosooxy, pyridyls, sulfhydryls, sulfides, disulfides, sulfinyls, thiols, sulfos, thiocyanates, isothiocyanates, carbonothioyls, phosphinos, phosphonos, phosphates, silyls, and Si(OH)₃.

16. The method of claim 15, wherein:
R¹-R⁶, R⁹-R¹¹, R¹³ and R¹⁶ are independently either hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, amines, azides, cyanates, nitrates, nitriles, nitros, thiols, and phosphates;
R¹² is CF₃;
R¹⁴ is SCH₃; and/or
R¹⁵ is NH₂.

17. The method of claim 7, wherein the allosteric RAF inhibitor comprises or consists of, or is selected from the group consisting of, any one of the following compounds, or equivalents thereof:
(a) N-(3-(4-(pyridin-3-yl)pyrimidin-2-ylamino)-4-methylphenyl)-4-((4-methylpiperazin-1-yl)methyl)benzamide;
(b) 1-(4-(2-(methylcarbamoyl)pyridin-4-yloxy)phenyl)-3-(4-chloro-3-(trifluoromethyl)phenyl)urea;
(c) 6-(4-(5-(3-(trifluoromethyl)phenylamino)-4H-1,2,4-triazol-3-yl)phenoxy)pyrimidin-4-amine;
(d) 6-(4-(5-(3-(trifluoromethyl)phenylamino)-4H-1,2,4-triazol-3-yl)phenoxy)pyrimidine-2,4-diamine;
(e) 6-(4-(5-(4-chloro-3-(trifluoromethyl)phenylamino)-4H-1,2,4-triazol-3-yl)phenoxy)pyrimidine-2,4-diamine;
(f) 6-(4-(5-(3-(trifluoromethyl)phenylamino)-4H-1,2,4-triazol-3-yl)phenoxy)-2-(methylthio)pyrimidin-4-amine;
(g) 5-(4-(2-(methylthio)pyrimidin-4-yloxy)phenyl)-N-(3-(trifluoromethyl)phenyl)-4H-1,2,4-triazol-3-amine;
(h) 6-(4-(5-(4-chloro-3-(trifluoromethyl)phenylamino)-4H-1,2,4-triazol-3-yl)phenoxy)-2-(methylthio)pyrimidin-4-amine;
(i) 4-(4-(5-(3-(trifluoromethyl)phenylamino)-4H-1,2,4-triazol-3-yl)phenoxy)-6-methoxypyrimidin-2-amine;
(j) 6-(4-(5-(3-(trifluoromethyl)phenylamino)-1,3,4-oxadiazol-2-yl)phenoxy)pyrimidine-2,4-Diamine;
(k) 6-(4-(5-(3-(trifluoromethyl)phenylamino)-1,3,4-oxadiazol-2-yl)phenoxy)-2-(methylthio)pyrimidin-4-amine;
(l) (Z)-3-((3,5-dimethyl-1 Hpyrrol-2-yl)methylene)indolin-2-one;
(m) 3-(3,5-dibromo-4-hydroxybenzylidene)-5-iodoindolin-2-one;
(n) 2-(methylsulfanyl)-6-[4-(2-{[3-(trifluoromethyl)phenyl]amino}-1H-imidazol-5-yl)phenoxy]pyrimidin-4-amine (also called “KG5”), or 2-(methylsulfanyl)-6-[4-(2-{[3-(trifluoromethyl)phenyl]amino}-1H-imidazol-5-yl)phenoxy]pyrimidin-4-amine (also called “K38-B, or KG38”), having the structure: 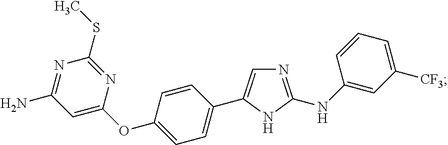 [SMILES: CSc1nc(N)cc(Oc2ccc(-c3cnc(Nc4cccc(C(F)(F)F)c4)[nH]3)cc2)n1]
(o) 6-(4-{5-[(2,3-dimethylphenyl)amino]-4H-1,2,4-triazol-3-yl}phenoxy)-2-(methylsulfanyl)pyrimidin-4-amine (also called “H3-11”), having the structure: 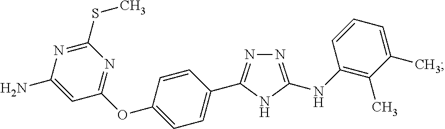 [SMILES: CSc1nc(N)cc(Oc2ccc(-c3nnc(Nc4cccc(C)c4C)[nH]3)cc2)n1]
(p) 2-(methylsulfanyl)-6-{4-[5-(5,6,7,8-tetrahydronaphthalen-1-ylamino)-4H-1,2,4-triazol-3-yl]phenoxy}pyrimidin-4-amine (also called “H3-21”), having the structure: 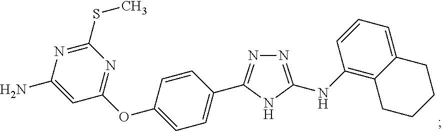 [SMILES: CSc1nc(N)cc(Oc2ccc(-c3nnc(Nc4cccc5c4CCCC5)[nH]3)cc2)n1]
(q) 6-(4-{5-[(3,4-dimethylphenyl)amino]-4H-1,2,4-triazol-3-yl}phenoxy)-2-(methylsulfanyl)pyrimidin-4-amine (also called “H3-9”), having the structure: 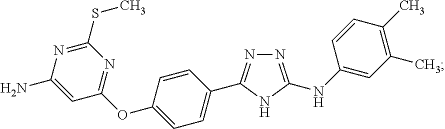 [SMILES: CSc1nc(N)cc(Oc2ccc(-c3nnc(Nc4ccc(C)c(C)c4)[nH]3)cc2)n1]
(v) 6-(4-{5-[(3,5-dimethylphenyl)amino]-4H-1,2,4-triazol-3-yl}phenoxy)-2-(methylsulfanyl)pyrimidin-4-amine (also called “H3-7”), having the structure: 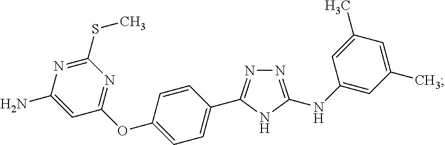 [SMILES: CSc1nc(N)cc(Oc2ccc(-c3nnc(Nc4cc(C)cc(C)c4)[nH]3)cc2)n1]
(s) 6-(4-{5-[(2,5-dimethylphenyl)amino]-4H-1,2,4-triazol-3-yl}phenoxy)-2-(methylsulfanyl)pyrimidin-4-amine (also called “H3-3”), having the structure: 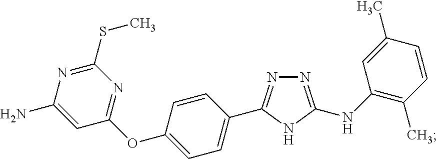 [SMILES: CSc1nc(N)cc(Oc2ccc(-c3nnc(Nc4cc(C)ccc4C)[nH]3)cc2)n1]
(t) 2-ethoxy-6-[4-(5-{[3-(trifluoromethyl)phenyl]amino}-4H-1,2,4-triazol-3-yl)phenoxy]pyrimidin-4-amine (also called “K1-72”), having the structure: 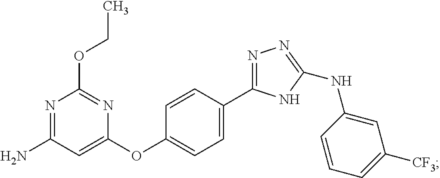 [SMILES: CCOc1nc(N)cc(Oc2ccc(-c3nnc(Nc4cccc(C(F)(F)F)c4)[nH]3)cc2)n1]
(u) a compound having a structural Formula I: 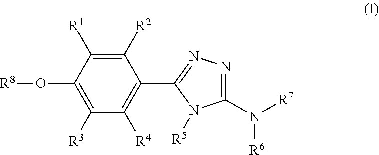
or a pharmaceutically acceptable salt or solvate thereof,
wherein:
R¹-R⁶ are independently selected from the group consisting of hydrogen, deuterium, alkyls, alkenyls, alkynyls, aryls, hetero-alkyls, hetero-alkenyls, hetero-alkynyls, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, thiols, sulfhydryls, sulfides, disulfides, sulfinyls, sulfos, thiocyanates, isothiocyanates, carbonothioyls, phosphinos, phosphonos, phosphates, silyls, and Si(OH)₃; and
R²-R⁸ are independently selected from the group consisting of cycloalkyl, cycloalkenyl, cycloalkynyl, aryl, heterocyle, and extended ring system;
(v) a genus of compounds as defined in step (u), with the proviso that the compound or specie is not a 6-(4-(5-(3-(trifluoromethyl)phenylamino)-4H-1,2,4-triazol-3-yl)phenoxy)-2-(methylthio)pyrimidin-4-amine (also can be designated “KG5”);
(w) a compound having structural Formula II: 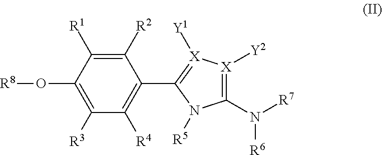
or a pharmaceutically acceptable salt or solvate thereof,
wherein:
X is independently N or C, and wherein when X is N then Y is absent;
R¹-R⁶, Y¹ and Y², are independently selected from the group consisting of hydrogen, deuterium, alkyls, alkenyls, alkynyls, aryls, hetero-alkyls, hetero-alkenyls, hetero-alkynyls, alkoxys, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, thiols, thioethers, thioethers, sulfhydryls, sulfides, disulfides, sulfinyls, sulfos, thiocyanates, isothiocyanates, carbonothioyls, phosphinos, phosphonos, phosphates, silyls, and Si(OH)₃; and
R⁷-R⁸ are independently selected from the group consisting of cycloalkyl, cycloalkenyl, cycloalkynyl, aryl, heterocyle, and extended ring system;
(x) a genus of compounds having a structural Formula II, wherein:
R⁷ is an aryl having the structure: 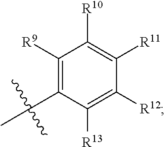
R⁸ is a heterocycle having the structure: 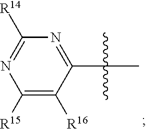
and
R⁹-R¹⁶ are independently selected from the group consisting of hydrogen, deuterium, alkyls, alkenyls, alkynyls, hetero-alkyls, hetero-alkenyls, hetero-alkynyls, alkoxys, halos, hydroxyls, carboxyls, carboxylates, aldehydes, haloformyls, esters, ethers, orthoesters, amines, azides, cyanates, isocyanates, nitrates, nitriles, isonitriles, nitrosos, nitros, nitrosooxy, pyridyls, sulfhydryls, sulfides, disulfides, sulfinyls, thiols, thioethers, sulfos, thiocyanates, isothiocyanates, carbonothioyls, phosphinos, phosphonos, phosphates, silyls, and Si(OH)₃;
(y) a genus of compounds having a structural Formula II, wherein:
R¹-R⁶, and R¹⁶ are independently selected from the group consisting of hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, amines, azides, cyanates, nitrates, nitriles, nitros, thiols, and phosphates;
R⁹-R¹³ are independently selected from the group consisting of hydrogen, deuterium, methyl and CF₃;
R¹⁴ is selected from the group consisting of (C₁-C₆)alkyls, (C₂-C₆)alkenyls, (C₂-C₆)alkynyls, hetero-(C₁-C₅)alkyls, hetero-(C₁-C₅)alkenyls, hetero-(C₁-C₅)alkynyls, alkoxys, ethers, carboxylic acid, amines, aldehyde, carbonyls, thiols, thioethers, esters, and azides, and
R¹⁵ is an amine; and/or
(z) the genus of compounds as defined in step (w) (based on structural Formula II) with the proviso that genus does not have (include) the compound or specie 6-(4-(5-(3-(trifluoromethyl)phenylamino)-4H-1,2,4-triazol-3-yl)phenoxy)-2-(methylthio)pyrimidin-4-amine (also can be designated “KG5”).

18. The method of claim 17, wherein:
R⁷ is an aryl having the structure: 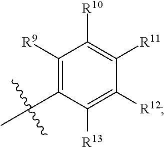
R⁸ is a heterocycle having the structure: 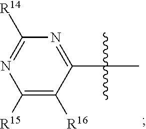
and/or
R⁹-R¹⁶ are independently selected from the group consisting of hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, orthoesters, amines, azides, cyanates, isocyanates, nitrates, nitriles, isonitriles, nitrosos, nitros, nitrosooxy, pyridyls, sulfhydryls, sulfides, disulfides, sulfinyls, thiols, sulfos, thiocyanates, isothiocyanates, carbonothioyls, phosphinos, phosphonos, phosphates, silyls, and Si(OH)₃.

19. The method of claim 18, wherein:
R¹-R⁶, R⁹-R¹¹, R¹³, and R¹⁶ are independently either hydrogen, deuterium, alkyls, alkenyls, alkynyls, hetero-alkyls, hetero-alkenyls, hetero-alkynyls, halos, hydroxyls, carboxyls, carboxylates, aldehydes, haloformyls, esters, ethers, amines, azides, cyanates, nitrates, nitriles, nitros, thiols, and phosphates;
R¹² is CF₃;
R¹⁴ is SCH₃; and/or
R¹⁵ is NH₂.
